SEQUENCE LISTING (109 sequences)

SEQ ID NO: 1 - protein, 22 aa, synthetic construct
  REGION 1..22: note = Peptide
QGQVQHLQAAFSQYKKVELFPK

SEQ ID NO: 2 - protein, 8 aa, synthetic construct
  REGION 1..8: note = peptide
KKVELFPK

SEQ ID NO: 3 - protein, 22 aa, synthetic construct
  REGION 1..22: note = peptide
CGQVCHLQCAFSCYKKCELFPK

SEQ ID NO: 4 - protein, 22 aa, synthetic construct
  REGION 1..22: note = peptide
QGQVCHLQCAFSCYKKCELFPK

SEQ ID NO: 5 - protein, 23 aa, synthetic construct
  REGION 1..23: note = peptide
QGQCCHLQCCFSQYKKCELFPKC

SEQ ID NO: 6 - protein, 23 aa, synthetic construct
  REGION 1..23: note = peptide
CGQCCHLQCCFCCYKKCELFPKC

SEQ ID NO: 7 - protein, 23 aa, synthetic construct
  REGION 1..23: note = peptide
CGCCCHLCCCFCCYKKCELFPKC

SEQ ID NO: 8 - protein, 23 aa, synthetic construct
  REGION 1..23: note = peptide
CGQVCHLQCAFSCYKKCELFPKC

SEQ ID NO: 9 - protein, 24 aa, synthetic construct
  REGION 1..24: note = peptide
CQGQVQHLQAAFSQYKKVELFPKC

SEQ ID NO: 10 - protein, 24 aa, synthetic construct
  REGION 1..24: note = peptide
CQGQVQHLQAAFSQYKKCELFPKC

SEQ ID NO: 11 - protein, 22 aa, synthetic construct
  REGION 1..22: note = peptide
QGQVQHLQCAFSCYKKCELFPK

SEQ ID NO: 12 - protein, 22 aa, synthetic construct
  REGION 1..22: note = peptide
QGQVQHLQAAFSCYKKCELFPK

SEQ ID NO: 13 - protein, 22 aa, synthetic construct
  REGION 1..22: note = peptide
QGQVQHLQAAFSCYKKVELFPK

SEQ ID NO: 14 - protein, 23 aa, synthetic construct
  REGION 1..23: note = peptide
CGQCCHLQCCFCQYKKCELFPKC

SEQ ID NO: 15 - protein, 23 aa, synthetic construct
  REGION 1..23: note = peptide
CGCCCHLCCCFCQYKKCELFPKC

SEQ ID NO: 16 - protein, 23 aa, synthetic construct
  REGION 1..23: note = peptide
CGQVCHLQCAFSQYKKCELFPKC

SEQ ID NO: 17 - protein, 23 aa, synthetic construct
  REGION 1..23: note = peptide
CGQVCHLQCAFSGYKKCELFPKC

SEQ ID NO: 18 - protein, 22 aa, synthetic construct
  REGION 1..22: note = peptide
CGQVCHLQCAFSCYKKCELFAK

SEQ ID NO: 19 - protein, 22 aa, synthetic construct
  REGION 1..22: note = peptide
CGQAQHLCAAFSQYKKVELFPK

SEQ ID NO: 20 - protein, 22 aa, synthetic construct
  REGION 1..22: note = peptide
CCQCQHCQCCCSCYKKVECFPC

SEQ ID NO: 21 - protein, 22 aa, synthetic construct
  REGION 1..22: note = peptide
QCQVQCCQACCSQYKCVELFPC

SEQ ID NO: 22 - protein, 22 aa, synthetic construct
  REGION 1..22: note = peptide
QGQVQHLQAACCQCCCVELFPC

SEQ ID NO: 23 - protein, 22 aa, synthetic construct
  REGION 1..22: note = peptide
QCQVQHLCCCFCCYKKVECCPC

SEQ ID NO: 24 - protein, 22 aa, synthetic construct
  REGION 1..22: note = peptide
QAQVQHACAACSAYKKCELFAC

SEQ ID NO: 25 - protein, 22 aa, synthetic construct
  REGION 1..22: note = peptide
QGCCQHCQCACSQCCKVECCCK

SEQ ID NO: 26 - protein, 22 aa, synthetic construct
  REGION 1..22: note = peptide
QCQCQCLQAAFCCYCKVELCPK

SEQ ID NO: 27 - protein, 22 aa, synthetic construct
  REGION 1..22: note = peptide
QGCCCHCQCAFCQYCACELCPK

SEQ ID NO: 28 - protein, 22 aa, synthetic construct
  REGION 1..22: note = peptide
QGCVCHLQAACSQYKKCELFPC

SEQ ID NO: 29 - protein, 22 aa, synthetic construct
  REGION 1..22: note = peptide
QGQVQHLQAACSQYKCAECFPK

SEQ ID NO: 30 - protein, 22 aa, synthetic construct
  REGION 1..22: note = peptide
QACVQHCQCCFCQAKAVELCPK

SEQ ID NO: 31 - protein, 22 aa, synthetic construct
  REGION 1..22: note = peptide
QGCCQCCQAACACCCACECFPC

SEQ ID NO: 32 - protein, 22 aa, synthetic construct
  REGION 1..22: note = peptide
CCQCCHCQCCFSQYCKVCCFPK

SEQ ID NO: 33 - protein, 22 aa, synthetic construct
  REGION 1..22: note = peptide
QGQVQHCQAAFSQYKCVCLFPK

SEQ ID NO: 34 - protein, 22 aa, synthetic construct
  REGION 1..22: note = peptide
CGCCQCLAAACSQYKAVCACPK

SEQ ID NO: 35 - protein, 22 aa, synthetic construct
  REGION 1..22: note = peptide
QGCVQHLQACCCQYCKVELCPK

SEQ ID NO: 36 - protein, 22 aa, synthetic construct
  REGION 1..22: note = peptide
QGCVQHLQAACSQYKKVELFPK

SEQ ID NO: 37 - protein, 22 aa, synthetic construct
  REGION 1..22: note = peptide
QGQVQALQAAFSQCAKVEAFPK

SEQ ID NO: 38 - protein, 22 aa, synthetic construct
  REGION 1..22: note = peptide
QGQVQHLQACFSQYKKCELFPK

SEQ ID NO: 39 - protein, 22 aa, synthetic construct
  REGION 1..22: note = peptide
QCCCQHCQAACSQAKACCCCPK

SEQ ID NO: 40 - protein, 22 aa, synthetic construct
  REGION 1..22: note = peptide
QCACACLCCACCCCKAAECFCK

SEQ ID NO: 41 - protein, 22 aa, synthetic construct
  REGION 1..22: note = peptide
QACVQALAACFSQCKCVCLCPC

SEQ ID NO: 42 - protein, 22 aa, synthetic construct
  REGION 1..22: note = peptide
CCQVQCCCAACCQYCKVECFPK

SEQ ID NO: 43 - protein, 22 aa, synthetic construct
  REGION 1..22: note = peptide
QGQVQHCCAAFSQYKKVELFPK

SEQ ID NO: 44 - protein, 22 aa, synthetic construct
  REGION 1..22: note = peptide
QGCVCHLCCCCCCCKCVECFCK

SEQ ID NO: 45 - protein, 22 aa, synthetic construct
  REGION 1..22: note = peptide
QCQAQCACAACCQYKKVACFPK

SEQ ID NO: 46 - protein, 22 aa, synthetic construct
  REGION 1..22: note = peptide
QGQCCCLQCACCQYCKCECFPC

SEQ ID NO: 47 - protein, 22 aa, synthetic construct
  REGION 1..22: note = peptide
CGQVCCCQACFSCCCKCCLFCK

SEQ ID NO: 48 - protein, 22 aa, synthetic construct
  REGION 1..22: note = peptide
QGCVACLQCAFSQYKKCAACPC

SEQ ID NO: 49 - protein, 22 aa, synthetic construct
  REGION 1..22: note = peptide
CGCVQHCQAAFCQYAKVCLFAK

SEQ ID NO: 50 - protein, 22 aa, synthetic construct
  REGION 1..22: note = peptide
QGCCCHCCCCFCQCCCVELFPC

SEQ ID NO: 51 - protein, 22 aa, synthetic construct
  REGION 1..22: note = peptide
QGQCQHCQAAFSQACKAELFPK

SEQ ID NO: 52 - protein, 22 aa, synthetic construct
  REGION 1..22: note = peptide
CCCVCCCQAACSAYCCCCACPK

SEQ ID NO: 53 - protein, 22 aa, synthetic construct
  REGION 1..22: note = peptide
CGCVQHLQACFSQCCCVELCCC

SEQ ID NO: 54 - protein, 22 aa, synthetic construct
  REGION 1..22: note = peptide
QCQAQHLQACASQYKKVALFPK

SEQ ID NO: 55 - protein, 22 aa, synthetic construct
  REGION 1..22: note = peptide
QGCCQHLCAAFSQYKCVELFPK

SEQ ID NO: 56 - protein, 22 aa, synthetic construct
  REGION 1..22: note = peptide
CGQVQHLCAAFSQYCKVEAFPC

SEQ ID NO: 57 - protein, 22 aa, synthetic construct
  REGION 1..22: note = peptide
QGACQCCCAAASQYACVELFPK

SEQ ID NO: 58 - protein, 22 aa, synthetic construct
  REGION 1..22: note = peptide
CGQVCHLQACFAQYKCVELFPK

SEQ ID NO: 59 - protein, 22 aa, synthetic construct
  REGION 1..22: note = peptide
CGCCAALCACCSCYKKACCFPA

SEQ ID NO: 60 - protein, 22 aa, synthetic construct
  REGION 1..22: note = peptide
QGQVQHLCACCSCYKKVCCCCC

SEQ ID NO: 61 - protein, 22 aa, synthetic construct
  REGION 1..22: note = peptide
CCCCQAACCACSCYCKVELFCK

SEQ ID NO: 62 - protein, 22 aa, synthetic construct
  REGION 1..22: note = peptide
QCQVQHLQAAFCQCKCCELCPK

SEQ ID NO: 63 - protein, 22 aa, synthetic construct
  REGION 1..22: note = peptide
QGQVQCLCAAFSQYKKCECFPK

SEQ ID NO: 64 - protein, 22 aa, synthetic construct
  REGION 1..22: note = peptide
CGCACALAACCSCCCCVALFPK

SEQ ID NO: 65 - protein, 22 aa, synthetic construct
  REGION 1..22: note = peptide
QAQAQHLCCAFCCCKKCELCPK

SEQ ID NO: 66 - protein, 22 aa, synthetic construct
  REGION 1..22: note = peptide
CGCCQHLQCCFCCYKCVCCFPK

SEQ ID NO: 67 - protein, 22 aa, synthetic construct
  REGION 1..22: note = peptide
CCQCQCLCAAFSCYKKVCCCPK

SEQ ID NO: 68 - protein, 22 aa, synthetic construct
  REGION 1..22: note = peptide
QACAQCLCAAFSQYAKVELFPK

SEQ ID NO: 69 - protein, 22 aa, synthetic construct
  REGION 1..22: note = peptide
QGQVQHLQCAFCCYKKVELFPK

SEQ ID NO: 70 - protein, 22 aa, synthetic construct
  REGION 1..22: note = peptide
QGQVQHLQACFSCYKCVELFPC

SEQ ID NO: 71 - protein, 22 aa, synthetic construct
  REGION 1..22: note = peptide
QGQVCCCCAAFCCCKCVELCCK

SEQ ID NO: 72 - protein, 22 aa, synthetic construct
  REGION 1..22: note = peptide
QGQVQHCQAAFSQYCKVELAAK

SEQ ID NO: 73 - protein, 22 aa, synthetic construct
  REGION 1..22: note = peptide
QGQVQHLQAAFSQYKKVCLFPK

SEQ ID NO: 74 - protein, 22 aa, synthetic construct
  REGION 1..22: note = peptide
QGQVQCLQAAFCQCKKVECFPK

SEQ ID NO: 75 - protein, 22 aa, synthetic construct
  REGION 1..22: note = peptide
QGCVQCLQCACCACKCCCLFCA

SEQ ID NO: 76 - protein, 22 aa, synthetic construct
  REGION 1..22: note = peptide
CACCQACCACFSCAKCAECFCK

SEQ ID NO: 77 - protein, 22 aa, synthetic construct
  REGION 1..22: note = peptide
QGQVQHCQAAFSQYKKCCLFPK

SEQ ID NO: 78 - protein, 22 aa, synthetic construct
  REGION 1..22: note = peptide
QAQVQHLQAAFSQCCCVELFCK

SEQ ID NO: 79 - protein, 22 aa, synthetic construct
  REGION 1..22: note = peptide
CCQCCCLCCAFCCCCKCELFPK

SEQ ID NO: 80 - protein, 22 aa, synthetic construct
  REGION 1..22: note = peptide
QGQVQCLCCCCSQYKKVECFPK

SEQ ID NO: 81 - protein, 22 aa, synthetic construct
  REGION 1..22: note = peptide
QGQVQHLQACFSAYKKVELFPK

SEQ ID NO: 82 - protein, 22 aa, synthetic construct
  REGION 1..22: note = peptide
QGQVCHCCCAFAQYKCACAFPK

SEQ ID NO: 83 - protein, 22 aa, synthetic construct
  REGION 1..22: note = peptide
QGQCQCLCAAFCCYKKVELCPK

SEQ ID NO: 84 - protein, 22 aa, synthetic construct
  REGION 1..22: note = peptide
QGQVQCLQAAASQYKKCELFCK

SEQ ID NO: 85 - protein, 22 aa, synthetic construct
  REGION 1..22: note = peptide
QGQVQCCQAACSQYKKVELFPC

SEQ ID NO: 86 - protein, 22 aa, synthetic construct
  REGION 1..22: note = peptide
AGCVQCLCCCCCCYAKVCAFPK

SEQ ID NO: 87 - protein, 22 aa, synthetic construct
  REGION 1..22: note = peptide
QGCCQCCCACCSQYKKVELFPC

SEQ ID NO: 88 - protein, 22 aa, synthetic construct
  REGION 1..22: note = peptide
ACQVQHCCCAFCCYCCVCCFCC

SEQ ID NO: 89 - protein, 22 aa, synthetic construct
  REGION 1..22: note = peptide
CCCCQHLCACFSCYCKVECFCK

SEQ ID NO: 90 - protein, 22 aa, synthetic construct
  REGION 1..22: note = peptide
QGQVQHLQCAFSCYKKVELFCC

SEQ ID NO: 91 - protein, 22 aa, synthetic construct
  REGION 1..22: note = peptide
CGQVQHLQAACSQYKCVELFCK

SEQ ID NO: 92 - protein, 22 aa, synthetic construct
  REGION 1..22: note = peptide
QCCVCCAQACFCQYKKCECCCA

SEQ ID NO: 93 - protein, 22 aa, synthetic construct
  REGION 1..22: note = peptide
CGAVQCCCACFCCCAKVECFCA

SEQ ID NO: 94 - protein, 22 aa, synthetic construct
  REGION 1..22: note = peptide
CGCCQCLCAACSCYCCVCLCCK

SEQ ID NO: 95 - protein, 22 aa, synthetic construct
  REGION 1..22: note = peptide
QGQVQCLQCAFSQAKCAEAFPA

SEQ ID NO: 96 - protein, 22 aa, synthetic construct
  REGION 1..22: note = peptide
QGCAQHACAAFSQYKAVELFPC

SEQ ID NO: 97 - protein, 22 aa, synthetic construct
  REGION 1..22: note = peptide
QGCVQHLQCACSQYKKCELACK

SEQ ID NO: 98 - protein, 22 aa, synthetic construct
  REGION 1..22: note = peptide
QGQCCHLQAAFSQYKKAELFPK

SEQ ID NO: 99 - protein, 22 aa, synthetic construct
  REGION 1..22: note = peptide
CCCCAHCACAFSQYKCACCCPC

SEQ ID NO: 100 - protein, 22 aa, synthetic construct
  REGION 1..22: note = peptide
CCQCQHLAACFAQYCACCCCCK

SEQ ID NO: 101 - protein, 22 aa, synthetic construct
  REGION 1..22: note = peptide
CCCVCCLQAACCCYKKCCLCCK

SEQ ID NO: 102 - protein, 22 aa, synthetic construct
  REGION 1..22: note = peptide
AGCCQCCCCAFCQYKKCCCFCC

SEQ ID NO: 103 - protein, 22 aa, synthetic construct
  REGION 1..22: note = peptide
QGQVQCLQCAFCCYKKVELFCC

SEQ ID NO: 104 - protein, 22 aa, synthetic construct
  REGION 1..22: note = peptide
CCQVCHLQCCAACYKKCCLFPC

SEQ ID NO: 105 - protein, 22 aa, synthetic construct
  REGION 1..22: note = peptide
CCQCQHAQCAFCACCCCACCCK

SEQ ID NO: 106 - protein, 22 aa, synthetic construct
  REGION 1..22: note = peptide
QGQVQHLQAAFSQCCACECFPK

SEQ ID NO: 107 - protein, 22 aa, synthetic construct
  REGION 1..22: note = peptide
QGACQALQAAFCQYKKVCAFCK

SEQ ID NO: 108 - protein, 22 aa, synthetic construct
  REGION 1..22: note = peptide
QCCCQHCQACCCCYKKCCCCPK

SEQ ID NO: 109 - protein, 22 aa, synthetic construct
  REGION 1..22: note = peptide
QGCVCHLQAACSQYKKVELFPC

(19) United States
(12) Patent Application Publication
(10) Pub. No.: US 2026/0021028 A1
(43) Pub. Date: Jan. 22, 2026

(54) Formulations, Kits, and Methods of Use Thereof for Coils, Curls, and Waves in Hair

(71) Applicant: Olaplex, Inc., Santa Barbara, CA (US)

(72) Inventors: Lavinia Popescu, Santa Barbara, CA (US); Mona Vaidya, Santa Barbara, CA (US); Matthew Cordes, Santa Barbara, CA (US)

(21) Appl. No.: 19/275,429

(22) Filed: Jul. 21, 2025

Related U.S. Application Data
(60) Provisional application No. 63/674,108, filed on Jul. 22, 2024.

(51) Int. Cl. A61K 8/64; A61K 8/41; A61Q 5/12

(52) U.S. Cl. CPC A61K 8/64; A61K 8/41; A61Q 5/12

(57) ABSTRACT

Formulations, kits, and methods for coils, curls, and waves in hair are disclosed. The formulations provide coils, curls, and/or waves that are visibly repaired, healthier, defined, and/or smoother. For example, the formulations disclosed herein provide curls that are shinier, bouncier, and/or more defined, with results that last through multiple washes, compared to when the curls are not treated with the formulations. In some forms, at least two formulations are used for treating curly hair to provide the afore-mentioned results, including a bond shaping formulation and a bond building formulation. The bond shaping formulation contains a peptide capable of penetrating within the cortex to help replenish keratin. The bond building formulation contains a binding agent capable of strengthening disulfide bonds. The bond shaping formulation and bond building formulation may be provided in a kit for defining curls. Improved methods of defining curls are also provided.

### CROSS-REFERENCE TO RELATED APPLICATIONS

This application claims the benefit of and priority to U.S. Provisional Application No. 63/674,108 filed Jul. 22, 2024, the entire content of which is incorporated herein by reference in its entirely.

### REFERENCE TO SEQUENCE LISTING

The Sequence Listing submitted as a text file named “OLA_201_PRO_ST26.xml” created on Jul. 22, 2024, and having a size of 136,587 bytes is hereby incorporated by reference pursuant to 37 C.F.R. § 1.834(c)(1).

### BACKGROUND

Keratin refers to the filament-forming proteins presenting specific physiochemical I properties, which can be extracted from the cornified layer of the epidermis. Keratin is the main protein in skin and makes up hair, nails, and the surface layer of the skin. Harsh chemicals and environmental influences such as UV and thermal radiation lead to lasting keratin damage to skin, hair, and nails.

A keratin protein is defined by a primary structure based on amino acid chains. The chains vary in number and sequence of amino acids, polarity, charge, and size. Small modifications in the keratin's amino acid sequence cause significant property modification, since these sequences determine the whole molecular structure and the nature of the bonds. The sulphur-containing amino acids, methionine and cysteine establish intra or intermolecular disulfide bonds. The role of disulphide bonds is important in keratin's structural integrity. The disulfide bonds can be broken by chemical treatment of the hair and over time result in serious long-lasting damage to the keratin.

The keratin intra-strand disulfide bonds result in characteristic waves, curls and the general body or appearance of one's hair. These characteristics of hair can be desirable or undesirable, resulting in an individual seeking hair treatment to change the natural body or shape of their hair. In order to limit or minimize the application of harsh chemicals used to straighten or shape hair into a desired conformation, there is a need to develop safer and more efficient alternative biologically-based compositions.

The object of this disclosure is to provide formulations for use in consumer products, cosmetic compositions, and/or hair treatments.

### BRIEF SUMMARY

This disclosure provides information related to methods of using one or more formulations to treat keratin fibers, in particular curly hair, for providing coils, curls, and/or waves that are visibly healthier, more defined, and/or smoother compared to a control treatment. In some forms, after being treated by the one or more formulations, the curls are shinier, bouncier, and/or more defined, optionally with results that last through multiple washes, compared to when the curls are not treated with the one or more formulations.

In some forms, at least two formulations are used to treat keratin fibers, in particular curly hair, which can be applied sequentially to the hair or combined and then applied to the hair as a mixture, or a combination thereof, to strengthen, reshape, and/or visibly restore a curl pattern, optionally a natural curl pattern, optionally after just one treatment. In some forms, the two or more formulations are provided to a user in a kit for treating keratin fibers, such as curly hair. In some forms, the two or more formulations are provided to the user separately.

In some forms, a bond shaping formulation containing a peptide is provided. The peptide of the bond shaping formulation is capable of penetrating within the cortex to help replenish keratin. Accordingly, in some forms, the bond shaping formulation can strengthen, shape, and/or lock the bonds responsible for providing curl shape and definition.

In a first aspect, the peptide in the bond shaping formulation has a substitution of at least one amino acid of SEQ ID NO:1 with a cysteine, wherein the peptide comprises at least 50% sequence identity to SEQ ID NO:1. The peptide has at least one cysteine substitution occurs at any one or more of a glutamine amino acid position, a valine amino acid position, an alanine amino acid position, and a serine amino acid position. The peptide forms an alpha helix and at least one of the cysteine substitutions is located on the outside region of the alpha helix.

In an embodiment, the cysteine substitution located in the region of SEQ ID NO:1 identified as SEQ ID NO:2. The cysteine substitution within the SEQ ID NO: 2 can be, for example, a V17C substitution relative to SEQ ID NO:1.

In another embodiment, the peptide has a substitution of an amino acid of SEQ ID NO:1, with a cysteine at H6C or Q13C, and optionally, but preferably an additional cysteine substitution on at least one additional amino acid at another position. The additional cysteine substitution can be from ≥1 to ≤11 amino acids relative to SEQ ID NO: 1.

In another aspect, the peptide has a substitution of at least 3 amino acids of SEQ ID NO: 1, and optionally these cysteine substitutions are on amino acids other than Q13C or H6C positions. In this aspect the cysteine substitutions are ≥3 and ≤11 amino acids relative to SEQ ID NO: 1.

In an embodiment, the cysteine is added at the peptide C-terminus, the N-terminus, or both the N-terminus and C-terminus relative to SEQ ID NO:1.

In a second aspect, the peptide is any one of SEQ ID NOS: 3-108 or a variant thereof with at least 70% sequence identity thereto.

In an embodiment, a bond shaping formulation containing a concentration of a plurality of the peptides disclosed herein increase hair strength relative to a formulation containing the same concentration of a plurality of peptides of SEQ ID NO: 1. This can be demonstrated by greater retention in the hair of the peptides disclosed here following repeated washings compared to retention of the same peptides in one washing relative to the same test under the same conditions for the retention of SEQ ID NO:1. The plurality of peptides can be used in a liquid bond shaping formulation to improve one or more properties, for example, hair's strength, visual properties, tactile properties, or a combination thereof. The keratin-bound peptide can be more resistant to washing than a keratin-bound peptide consisting of SEQ ID NO: 1, particularly following damage such as by bleaching one or more time. The liquid bond shaping formulation can be a shampoo, conditioner, oil, serum, or mask. The peptide can be found in a concentration of about 0.01% to about 0.1% w/w of the bond shaping formulation.

In an embodiment, the hair is treated by applying the liquid bond shaping formulation containing the peptide subsequent to application of a hair waving formulation, hair straightening formulation, hair coloring formulation, or hair bleaching formulation to the hair, resulting in a decrease in hair breakage by at least about 5%, 10%, 20%, 30%, 40%, or 50% as compared to when the hair is not treated with the composition following the application of a hair waving formulation, hair straightening formulation, hair coloring formulation, or hair bleaching formulation. As described, the composition can be found as part of a kit.

In some forms, a bond building formulation containing an active agent capable of strengthening disulfide bonds is provided. Accordingly, in some forms, the bond building formulation can improve hair structure and/or hair strength to provide visibly healthier curls.

Optionally, one or more additional formulations are provided, which can be used in combination with the bond shaping formulation and/or the bond building formulation, in any desired order and any desired fashion (used separately or mixed with the bond shaping formulation and/or bond building formulation). The one or more additional formulations may enhance the treatment effects of the bond shaping formulation and/or the bond building formulation. In some forms, a third formulation is provided, optionally the third formulation is an enhancing formulation that can be used together with the bond shaping formulation and the bond building formulation, in any desired order and any desired fashion.

### DEFINITIONS

The terms used in this specification generally have their ordinary meanings in the art, within the context of the invention, and in the specific context where each term is used. Certain terms are discussed below, or elsewhere in the specification, to provide additional guidance to the practitioner in describing the compounds, compositions, and methods and how to make and use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. The use of examples anywhere in this specification, including examples of any terms discussed herein, is illustrative only, and in no way limits the scope and meaning of any exemplified term. Likewise, the examples presented are considered non-limiting.

As used herein one embodiment,” “an embodiment,” “an aspect,” “one aspect,” indicate inclusion of a particular feature, structure, or characteristic. This is not an inclusion of all possible features, structures, or characteristics. Moreover, such phrases are not necessarily referring to the same aspect or embodiment. Further, when a particular feature, structure, or characteristic is described in connection with an aspect or embodiment, it is submitted that it is within the knowledge of one skilled in the art to affect such feature, structure, or characteristic in connection with other aspects or embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

As used herein, “about” or “approximately” shall generally mean within 20 percent, preferably within 10 percent, and more preferably within 5 percent of a given value or range. Other than in the operating examples, or where otherwise indicated, all numbers expressing quantities of ingredients and/or reaction conditions are to be understood as being modified in all instances by the term “about”.

The recitation of numerical ranges by endpoints includes all numbers within that range. By way of non-limiting example, the range “1 to 5” would also include the values 1, 1.5, 2, 2.75, 3, 3.8, 4, 5.

As used in the specification and the appended claims, the singular forms “a”, “an”, and “the” include plural referents unless the context clearly dictates otherwise.

As used in this specification and the appended claims, the term “or” is generally employed in its sense including “and/or” unless the context clearly indicates otherwise.

As used herein, “hair keratin disulfide” refers to thiol residues naturally existing in hair keratin that form disulfide bonds. The formation of new disulfide bonds between thiol residues and peptides can result in straightened hair, shaped hair, or hair with a desired flow and body.

As used herein “bonding efficiency” refers to the ability to form a chemical bond, such as between the sulfur atoms in two cysteine residues.

As used herein “replacement” refers to the substitution of one amino acid in a protein sequence with another.

As used herein “addition” refers to the insertion of an amino acid between two other amino acids in a protein sequence.

As used herein “residue” refers to an amino acid.

As used herein “bioconjugation site” refers to a chemically reactive moiety that allows attachment to another molecule.

As used herein a “vector” refers to a DNA molecule that allows transcription of the encoded genetic information.

### BRIEF DESCRIPTION OF THE DRAWINGS

FIG. 1: Results of Example 1, from left to right, hair was treated with: (1) untreated, (2) SEQ ID NO. 3, (3) SEQ ID NO. 4, (4) SEQ ID NO. 5, (5) SEQ ID NO. 6, (6) SEQ ID NO. 7, (7) SEQ ID NO. 8.

FIG. 2: Results of Example 2, from left to right, hair was treated with: (1) untreated, (2) SEQ ID NO. 3, (3) SEQ ID NO. 4, (4) SEQ ID NO. 5, (5) SEQ ID NO. 6, (6) SEQ ID NO. 7, (7) SEQ ID NO. 8.

FIG. 3: Results of Example 3, Hair was untreated (left) or treated using the full procedure described in Example 3 (right).

FIG. 4: Results of Example 4, Hair was untreated (left) or treated using the full procedure described in Example 4 (right).

FIG. 5: Results of Example 5, Hair was untreated (left) or treated using the full procedure described in Example 5 (right).

FIG. 6 (a): Results of Example 6, From left to right, hair was treated with: (1) untreated, (2) full treatment without SEQ ID NO. 7, (3) full treatment with SEQ ID NO. 7.

FIG. 6 (b): Results of Example 6, Break force comparison of hair samples (2) and (3) from FIG. 6a.

FIG. 7 (a): Results of Example 7, demonstrating the effect of varying the peptide concentration.

FIG. 7 (b): Results of Example 7, demonstrating the effect of varying the peptide binding time.

FIG. 7 (c): Results of Example 7, demonstrating the effect of varying the number of peptide applications.

FIG. 7 (d): Results of Example 7, demonstrating the effect of varying the number of shampoos at the end of treatment.

FIG. 8 (a): Results of Example 8, demonstrating the break force comparison of treated and untreated virgin hair with different peptides. Dotted bars represent the results after 1 shampoo, and bars with diagonal lines represent the results after 10 shampoos.

FIG. 8 (b): Results of Example 8, demonstrating the break force comparison of treated and untreated bleached virgin hair with different peptides. Dotted bars represent the results after 1 shampoo, and bars with diagonal lines represent the results after 10 shampoos.

FIG. 9 (a): Results of virgin hair after application of fluorescent peptide and shampooing 1 to 10 times as described in Example 9. Dotted bars represent the results after 1 shampoo, and bars with horizontal lines represent the results after 10 shampoos.

FIG. 9(b): Results of bleached hair after application of fluorescent peptide and shampooing 1 to 10 times as described in Example 9. Dotted bars represent the results after 1 shampoo, and bars with horizontal lines represent the results after 10 shampoos.

FIG. 9(c): Comparison of percent decrease in fluorescence intensity between 1 and 10 shampoos for peptides with and without Q13C substitution as described in Example 9. Dotted bars represent the results with the Q13C substitution, and bars with diagonal lines represent the results without the Q13C substitution.

FIG. 10(a): Results of Example 10, demonstrating the percent decrease in fluorescence intensity between 1 and 10 shampoos for peptides that contain a cysteine substitution at the specified amino acid position (bars with horizontal lines) and peptides that do not contain a cysteine substitution at this position (bars with diagonal lines). Error bars represent a 95% confidence interval.

FIG. 10(b): Results of Example 10, demonstrating the percent decrease in fluorescence intensity between 1 and 10 shampoos for peptides that contain a Q13C substitution and a total number of cysteine substitutions greater than 1 (horizontal line) and peptides that either do not contain a Q13C substitution or have ≤1 substitutions (bar with diagonal lines). Error bars represent 95% confidence intervals.

FIG. 10(c): Results of Example 10, demonstrating the percent decrease in fluorescence intensity between 1 and 10 shampoos for peptides that contain a H6C substitution and a total number of cysteine substitutions greater than 1 (horizontal line) and peptides that either do not contain a H6C substitution or have ≤1 substitutions (bar with diagonal lines). Error bars represent 95% confidence intervals.

FIG. 10(d): Results of Example 10, demonstrating the percent decrease in fluorescence intensity between 1 and 10 shampoos for peptides that do not contain a Q13C or a H6C substitution and a total number of cysteine substitutions greater than the specified number of substitutions (bars with horizontal lines) and all other peptides from Example 10 (bars with diagonal lines). Error bars represent 95% confidence intervals. FIGS. 11 (a)-(d): Sequences (SEQ ID NO:1-218) tested and described in FIGS. 1-10.

### DETAILED DESCRIPTION

While the concepts of the present disclosure are susceptible to various modifications and alternative forms, specific aspects and embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims. It will be apparent to one skilled in the art that various changes or equivalents may be made without departing from the scope herein.

Provided herein are methods of using one or more formulations to treat keratin fibers, in particular curly hair, for providing coils, curls, and/or waves that are visibly repaired, healthier, defined, and/or smoother. In some forms, after being treated with the one or more formulations, the curls are shinier, bouncier, and/or more defined, optionally with results that last through multiple washes, compared to when the curls are not treated with the one or more formulations. The treatment results can be evaluated by visual inspection and/or one or more tests, such as curl droop test (see Section I in Example 11 below), anti-breakage test (see Section II in Example 11 below), anti-frizz test (see Section III in Example 11 below), and/or wet combing test (see Section IV in Example 11 below).

In some forms, at least two formulations are used to treat keratin fibers, in particular curly hair, which can be applied sequentially to the hair or combined and then applied to the hair as a mixture or a combination thereof, to repair, strengthen, reshape, and/or visibly restore a curl pattern, optionally after just one treatment. In some forms, the two or more formulations are provided in a kit for treating keratin fibers, such as curly hair. In some forms, the two or more formulations are provided separately.

In some forms, a bond shaping formulation and/or a bond building formulation are provided for treating curly hair, to provide coils, curls, and/or waves that are healthier, defined, and/or smoother based on visual inspection and/or test(s), such as curl droop test, anti-breakage test, anti-frizz test, and/or wet combing test. For example, hair treated with the bond shaping formulation and bond building formulation shows a lower percentage of curl droop (i.e., a higher percentage of curl retention), as compared to a control treated with a non-conditioning shampoo, such as 15% SLES solution (see Section I in Example 11 below). For example, hair treated with the bond shaping formulation and bond building formulation shows a higher percentage in breakage reduction, as compared to a control treated with a non-conditioning shampoo, such as 15% SLES solution (see Section II in Example 11 below). More specifically, hair treated with the bond shaping formulation and bond building formulation can reduce hair breakage by at least about 5, 10, 15, 20, 25, 30, 35, 40, 45, or 50% or higher compared to a control treated with a non-conditioning shampoo, such as 15% SLES solution. The reduction in breakage can be tested by a suitable method, such as described in Section II in Example 11 below. For example, hair treated with the bond shaping formulation and bond building formulation shows a higher percentage in frizz reduction, as compared to a control treated with a non-conditioning shampoo, such as 15% SLES solution (see Section III in Example 11 below). More specifically, hair treated with the bond shaping formulation and bond building formulation can reduce frizz by at least 30%, at least 40%, at least 50%, at least 60%, or at least 70% following one treatment, compared to a control treated with a non-conditioning shampoo, such as 15% SLES solution. The reduction in frizz can be tested by a suitable method, such as described in Section III in Example 11 below. For example, hair treated with the bond shaping formulation and bond building formulation shows a lower wet combing force, as compared to a control treated with a non-conditioning shampoo, such as Suave non-conditioning shampoo (see Section IV in Example 11 below).

Additionally, the bond shaping formulation and/or a bond building formulation are safe for use on color-treated hair, as demonstrated by a standard color retention test. For example, the color change of hair treated with the bond shaping formulation and bond building formulation is statistically the same as a control treated with a non-conditioning shampoo and water, such as 15% SLES solution and water (see Section V in Example 11 below).

Optionally, one or more additional formulations are also provided that can be used in combination with the bond shaping and/or bond building formulation, in any desired order and any desired fashion. The formulation(s) can be provided as part of a kit, such as a kit for shaping curls.

### A. Exemplary Bond Shaping Formulations

In some forms, a bond shaping formulation is provided for treating curly hair. The bond shaping formulation contains a peptide capable of penetrating within the cortex to help replenish keratin. Accordingly, in some forms, the bond shaping formulation can lock and/or shape disulfide bonds into place for enhanced curl definition and/or shape.

Such peptides and compositions containing such peptides (also referred to herein as “bond shaping formulation”) can typically bind to one or more thiol residues naturally existing in hair keratin, thereby allowing for the formation of new disulfide bonds between hair keratin and the peptides. Provided herein, for example, is a method to provide a long-lasting shape flow and body to hair accomplished by: 1. mechanically structuring hair keratin into a desired shape (i.e., permanent wave) or into a straightened formation; 2. breaking the hair keratin natural disulfide bonds; and 3. applying the peptides to the hair keratin forming new disulfide bonds between the hair keratin and peptides. In some other instances, methods of treating hair involve applying the peptides to hair prior to and/or following a coloring or bleaching treatment, where the keratin in the treated hair forms new disulfide bonds between the hair keratin and peptides applied. In some instances, methods of treating hair can involve applying the peptides to hair, such as for strengthening hair and/or reducing or preventing breakage in hair, where the peptides can form part of a cosmetic composition, such as in the form of a shampoo, conditioner, oil, or mask, etc. The application of peptides to hair can produce new disulfide bonds between the hair keratin and peptides, which may be part of a cosmetic composition, and are believed to improve one or more properties of the hair, such as but not limited to, increasing hair strength, resistance to breakage, tactile feel (i.e., softness), visual properties (i.e., sheen), as compared to hair prior to treatment with the peptides.

Hair can sustain damage from environmental factors like aging, washing, coloring and styling. Repeated washing results in lifted cuticles, while heat damage from drying, and chemical damage from coloring, bleaching, straightening, or curling hair often leads to dehydrated, easily broken, damaged and frizzy hair. Hair frizz, whether naturally present in the subject's hair or the result of environmental damage, generally refers to hair in which all strands are not uniformly aligned. The degree of hair frizz (or frizziness) is defined by the alignment of each hair strand relative to the surrounding hair strands. The present disclosure is also useful for shaping and straightening frizzy hair strands into a desired absolute directionality defined straightness or waviness of a population of individual (or bulk) hair strands. The present disclosure is also useful for shaping and straightening frizzy hair strands into a desired absolute directionality defined straightness or waviness of a population of individual (or bulk) hair strands.

The peptide sequences have at least one amino acid difference from SEQ ID NO: 1 and are typically capable of binding to keratin. It is believed that the amino acid sequence KKVELFPK (SEQ ID NO: 2.), is responsible for SEQ ID NO: 1's binding to hair keratin. In the disclosed peptides, any one or more of the amino acids in the amino acid sequence of SEQ ID NO: 1 and SEQ ID NO:2, can be replaced or substituted with cysteine.

In some examples, the total cysteine content of a disclosed peptide is less than 50% of SEQ ID NO. 1, less than 45% of SEQ ID NO. 1, less than 40% of SEQ ID NO. 1, less than 35% of SEQ ID NO. 1, less than 30% of SEQ ID NO. 1, less than 25% of SEQ ID NO. 1, less than 20% of SEQ ID NO. 1, less than 15% of SEQ ID NO. 1, less than 10% of SEQ ID NO. 1, less than 9% of SEQ ID NO. 1, less than 8% of SEQ ID NO. 1, less than 7% of SEQ ID NO. 1, less than 6% of SEQ ID NO. 1, or less than 5% of SEQ ID NO. 1.

Peptides having the amino acid sequence of any one of SEQ ID NOS: 3.-SEQ ID NOS: 17., SEQ ID NOS: 18-SEQ ID NOS: 109, fragments and variants of any one of SEQ ID NO: 1 and SEQ ID NOS: 3.-SEQ ID NOS: 17., SEQ ID NOS: 18-SEQ ID NOS: 109, are provided and can be used individually or in combination in a hair composition such as those disclosed herein, known in the art, or designed by one of skill in the art. The fragments and variants can have, for example, at least 50%, 55%, 60%, 65%, 70%, 75%, 80%, 85%, 90%, or 95% sequence identity to any one of more of SEQ ID NO: 1 and SEQ ID NOS: 3.-SEQ ID NOS: 17., SEQ ID NOS: 18-SEQ ID NOS: 109. The variants can include one or more amino acid substitution(s), addition(s), deletion(s), or a combination thereof relative to the reference peptide. The peptides typically include at least 1, preferably 2, 3, or more cysteines.

In some embodiments, the peptide is 10-50 amino acids in length, or any subrange there between inclusive, or any specific integer there between inclusive, including, but not limited to 10-45, 10-40, 10-35, 10-30, 10-29, 10-28, 10-27, 10-26, 10-25, 10-24, 10-23, 10-22, 10-21, 10-20, 10-19, 10-18, 10-17, 10-16, 10-15, 15-20, 15-21, 15-22, 15-23, 15-24, 15-25, 15-26, 15-27, 15-28, 15-29, 15-30, 18-27, 19-26, 20-25, or 21-24 amino acids inclusive, or 10, 11, 12, 13, 14, 15, 16, 17, 18, 19, 20, 21, 22, 23, 24, 25, 26, 27, 28, 29, or 30 amino acids.

In some embodiments, the peptide is SEQ ID NO: 8 or a variant thereof with at least 70% sequence identity thereto. In some embodiments, the peptide is SEQ ID NO: 8.

Typically, the peptides can bind to hair and/or a keratin protein thereof. Preferably the peptides show improved binding and/or retention to hair, relative to a control peptide, such as SEQ ID NO: 1. Binding and/or retention of the peptide(s) can be measured, for example, by fluorescently labelling the peptide(s) and applying them to hair or keratin protein thereof and allowing them to bind to the hair and/or keratin protein thereof and subsequently subjecting the hair to repeated washings (i.e., shampoos). Comparison and statistical analysis of fluorescence imaging of the labeled peptides on the hair prior to washing and following at least one, 5, 10, 15, 20, 25, or 30 washings allows for determination of the degree of retention of the peptide(s) to the hair. In some embodiments, the hair is damaged hair. For example, in some embodiments, prior to testing binding and/or retention to the hair, the hair is damaged, such as by subjecting the hair to repeated bleaching treatments as described in Examples 8 and 9.

In some instances, binding and/or retention to the hair is demonstrated by fluorescently labeling a peptide and applying the labeled peptide to the hair. Next the hair is washed one or more times and retention of the labeled peptide to washing is measured, such as by fluorescence imaging, and compared at one washing and at ten washings, optionally at 15 washings, at 20 washings, or more. In some instances, there is no significant difference (less than 5% reduction in fluorescence) in retention (the amount of peptide retained in the hair) after one washing compared to retention after ten washings. In some instances, the percent reduction in retention is less than about 50%; when comparing fluorescence detected after one washing to the fluorescence detected after ten washings. The same washing test can be conducted for any of the peptides described herein to determine the degree of retention of the peptides in the hair, and their retention ability. In some embodiments, prior to testing binding and/or retention to the hair, the hair is damaged, such as by subjecting the hair to repeated bleaching treatments as described in Examples 8 and 9.

In some embodiments, the peptides can be used at a reduced concentration to achieve the same or an improved results, as compared to a control such as SEQ ID NO: 1. For example, in some embodiments, the concentration of the peptide(s) is about less than 95% wt, about less than 90% wt, about less than 85% wt, about less than 80% wt, about less than 75% wt, about less than 70% wt, about less than 65% wt, about less than 60% wt, about less than 55% wt, about less than 50%, wt about less than 45% wt, about less than 40% wt, about less than 35% wt, about less than 30% wt, about less than 25% wt, about less than 20% wt, about less than 15% wt, about less than 10% wt, about less than 5% wt, about 0.01 to about 0.1% wt of the effective concentration of the peptide corresponding to SEQ ID NO.1 used for hair straightening or shaping, or used for other common hair treatments, and/or binding to keratin or hair and/or improving the strength, look, or feel of hair.

In some embodiments, the peptide has one or more of the amino acid residues corresponding to SEQ ID NO 2 of SEQ ID NO. 1 replaced with a cysteine amino acid residue. To derive the peptide one or more glutamine (Q) amino acid residues of SEQ ID NO. 1, in any combination, replaced or substituted with a cysteine, one or more valine (V) amino acid residues of SEQ ID NO. 1, in any combination, replaced or substituted with a cysteine, one or more alanine (A) amino acid residues of SEQ ID NO. 1, in any combination, replaced or substituted with a cysteine, and/or one or more serine(S) amino acid residues of SEQ ID NO. 1, in any combination, replaced or substituted with a cysteine. In some embodiments, one or more of glutamine, valine, alanine, and serine amino acid residues of SEQ ID NO. 1, in any combination, each replaced or substituted with a cysteine.

In some embodiments, the peptide includes the addition of cysteine residues to either or both of the N- or C-terminus of any of SEQ ID NOS. 1 or 3-109. In some embodiments, there are ten cysteine amino acid residues added to the N-terminus of any of SEQ ID NOS. 1 or 3-17 and 18-109, or a variant thereof, nine cysteine amino acid residues added to the N-terminus of any of SEQ ID NOS. 1 or 3-16, and 18-109, or a variant thereof, eight cysteine amino acid residues added to the N-terminus of any of SEQ ID NOS. 1 or 3-17, and 18-109, or a variant thereof, seven cysteine amino acid residues added to the N-terminus of any of SEQ ID NOS. 1 or 3-17 and 18-109, or a variant thereof, six cysteine amino acid residues added to the N-terminus of any of SEQ ID NOS. 1 or 3-17 and 18-109, or a variant thereof, five cysteine amino acid residues added to the N-terminus of any of SEQ ID NOS. 1 or 3-17 and 18-109, or a variant thereof, four cysteine amino acid residues added to the N-terminus of any of SEQ ID NOS. 1 or 3-17 and 18-109, or a variant thereof, three cysteine amino residues added to the N-terminus of any of SEQ ID NOS. 1 or 3-17., or a variant thereof, two cysteine amino acid residues added to the N-terminus of any of SEQ ID NOS. 1 or 3-17 and 18-109, or a variant thereof, or one cysteine amino acid residue is added to the N-terminus of any of SEQ ID NOS. 1 or 3-17 and 18-109, or a variant thereof. In some embodiments, there are ten cysteine amino acid residues added to the C-terminus of any of SEQ ID NOS. 1 or 3-17 and 18-109, or a variant thereof, nine cysteine amino acid residues added to the C-terminus of any of SEQ ID NOS. 1 or 3-17 and 18-109, or a variant thereof, eight cysteine amino acid residues added to the C-terminus of any of SEQ ID NOS. 1 or 3-17 and 18-109, or a variant thereof, seven cysteine amino acid residues added to the C-terminus of any of SEQ ID NOS. 1 or 3-17 and 18-109, or a variant thereof, six cysteine amino acid residues added to the C-terminus of any of SEQ ID NOS. 1 or 3-17 and 18-109, or a variant thereof, five cysteine amino acid residues added to the C-terminus of any of SEQ ID NOS. 1 or 3-17 and 18-109, or a variant thereof, four cysteine amino acid residues added to the C-terminus of any of SEQ ID NOS. 1 or 3-17 and 18-109, or a variant thereof, three cysteine amino residues added to the C-terminus of any of SEQ ID NOS. 1 or 3-17 and 18-109, or a variant thereof, two cysteine amino acid residues added to the C-terminus of any of SEQ ID NOS. 1 or 3-17 and 18-109, or a variant thereof, or one cysteine amino acid residue is added to the C-terminus of any of SEQ ID NOS. 1 or 3-17 and 18-109, or a variant thereof. Any ten or less of cysteine amino acid residues added to the C-terminus of SEQ ID NO. 1 can be in any combination with any ten or less of cysteine amino acid residues added to the N-terminus of SEQ ID NO. 1.

Peptides of SEQ ID NO: 3 through SEQ ID NO: 17 and 18-109 or a variant thereof were modified with cysteine substitutions at various positions relative to SEQ ID NO: 1. Peptides having a Q13C or H6C and at least one additional cysteine substitution showed increased retention to hair after 10 shampoos when compared to peptides not having at least a Q13C or H6C substitution. The location of the amino acid cysteine substitution on the outside region of the peptide alpha helix in a position similar to the Q13C and H6C positions may play a role in the increased retention to hair after multiple shampoos.

The peptides as disclosed can be combined with one or more cosmetically acceptable carriers (i.e., water or aqueous solutions) and/or cosmetically acceptable excipients that are considered safe and effective to human hair and/or human scalp and may be administered to an individual's hair without causing undesirable biological side effects, such as burning, itching, and/or redness, or similar adverse reactions. Herein, cosmetically acceptable carriers and cosmetically acceptable excipients, together, are referred to as cosmetically acceptable ingredients. The compositions may further contain an excipient that renders the formulations neutral pH, or a pH from about 5 to about 8. The composition may be a hair composition of any suitable form. Non-limiting examples may be in the form of low to moderate viscosity liquids, lotions, milks, oils, masks, mousses, sprays, gels, creams, shampoos, conditioners, and the like. In some forms, the bond shaping formulation is a leave-in formulation in a suitable form, such as a spray or conditioner. When provided as a leave-in formulation, the bond shaping formulation can be applied to hair, and the hair is not rinsed or washed for at least 24 hours, at least 36 hours, at least 48 hours, at least 60 hours, or at least 72 hours. Suitable excipients, such as those listed below, can be included or excluded from the hair care composition, depending on the form of use of the composition (e.g., hair spray, cream, conditioner, or shampoo).

The peptides as disclosed can be combined with one or more cosmetically acceptable ingredients in the bond shaping formulation. One of skill in the art would determine the proper ratio and combination of ingredients, including, but not limited to, surfactants, preservatives, chelating agents (such as ethylenediaminetetraacetic acid; trisodium ethylenediamine disuccinate; citric acid), preservative, vitamins (such as vitamin E or C), proteins, film formers, detergents, resins, hair fixatives, opacifying agents, volatiles, propellants, acidifying agents (such as ascorbic acid, citric acid), alkalizing agents (such as sodium hydroxide, sodium carbonate), pH adjustors (such as citric acid, sodium hydroxide, phosphoric acid, etc.), neutralizing agents, hydrolyzing agents, liquid vehicles, carriers, anti-frizz agents, absorbents, emulsifiers, softeners, solubilizers, moisturizers, humectants, hydrolyzed proteins, reconstructors, acidifiers, acidity regulators, detanglers, polymers, glossers, lubricants, sequestrants, antistatic agents, sunscreens, thermal protectors, conditioners, buffers, stabilizers (such as, hydroxyethylcellulose), thickeners, salts, emollients, antioxidants, alcohols, polysorbates, PEGs, polyquaternium polymers (such as, polyquaternium-7, polyquaternium-11, polyquaternium-113), quarternary ammonium compounds, fragrances, dyes or colors, oils, esters, fatty acids, bioactive additives, silicones, and an aqueous carrier. In some instances, in a bond shaping formulation, the peptides are present in combination with water, hair conditioning agent(s), pH adjustors and/or buffering agent(s), chelating agent(s), preservative(s), surfactant(s), moisturizers or lubricating agent(s), and emollients. In some instances, in a bond shaping formulation, the peptides are present in combination with water, stabilizer(s), hair conditioning agent(s), pH adjustors and/or buffering agent(s), emulsifier(s), chelating agent(s), preservative(s), and fragrances. The cosmetically acceptable excipients, as may be present, can typically be present in an amount ranging from about 10 wt % to about 99.99 wt % of the composition, about 40 wt % to about 99 wt %, or from about 80 wt % to about to about 99 wt %. More specific exemplary bond shaping formulations are described in the Examples below.

It is understood that certain molecules can fall under more than one category of cosmetically acceptable ingredients. For example, citric acid included in the formulation is considered a chelating agent or a pH adjustor, or both, depending on the specific needs and other components in the formulation. For example, glyceryl caprylate included in the formulation is considered an emollient, a surfactant, or a preservative, or a combination thereof, depending on the specific needs and other components in the formulation. For example, certain surfactants, such as polyglyceryl-6 oleate and sodium surfactin, are also emulsifiers.

In some embodiments, the composition includes an effective amount of peptide(s) to improve one or more aspects of hair's strength, visual appearance (i.e., sheen), or tactile properties (i.e., feel), or alter its shape or body compared to, for example, untreated hair or hair treated with an equivalent amount of a control peptide such as SEQ ID NO: 1.

In some embodiments, the composition includes an effective amount of peptide(s) to improve hair's strength, reduce frizziness, and/or improve curl definition, compared to, for example, untreated hair or hair treated with an equivalent amount of a control peptide such as SEQ ID NO: 1.

Hair breakage is a significant problem encountered during coloring, bleaching, and other hair treatments. In some instances, the compositions described herein can improve hair quality by decreasing hair breakage when the hair is subjected to treatments, such as coloring, bleaching, straightening or permanent waving. In some instances, use of the disclosed peptide(s) and compositions thereof can reduce hair breakage by at least about 5, 10, 15, 20, 25, 30, 35, 40, 45, or 50% or higher after treatment with the peptides, as compared to untreated hair from the same individual. Standard methods of measuring hair breakage are known and involve use of repeated hair grooming experiments-whereby the number of broken hair fibers are recorded as a function of repeated combing/brushing strokes. For example, see T.A.Evans & K.Park, A statistical analysis of hair breakage. II. Repeated grooming experiments. J.Cosmet. Sci., 61, 439-455, 2010.

The peptide(s) disclosed herein may contain a lipid conjugated to the site for bioconjugation. For example, the site for bioconjugation can be the amino acid sequence AKT, where the lipid is conjugated to the lysine of the amino acid sequence AKT. Any suitable lipids can be conjugated to peptides disclosed herein. Examples include, but are not limited to, myristoleic acid/alcohol/amine, palmitoleic acid/alcohol/amine, sapienic acid/alcohol/amine, oleic acid/alcohol/amine, elaidic acid/alcohol/amine, vaccenic acid/alcohol/amine, linoleic acid/alcohol/amine, linoelaidic acid/alcohol/amine, alpha linolenic acid/alcohol/amine, arachidonic acid/alcohol/amine, eicosapentaenoic acid/alcohol/amine, erucic acid/alcohol/amine, caprylic acid/alcohol/amine (octanoic acid/alcohol/amine), lauric acid/alcohol/amine, myristic acid/alcohol/amine, palmitic acid/alcohol/amine, lignoceric acid/alcohol/amine, arachidic acid/alcohol/amine, stearic acid/alcohol/amine, and sphingolipids including ceramide, sphingosine, sphingomyelin, alpha cerebroside, gangliosides, sulfatides, and phytosphingosine. The peptide compositions disclosed herein may be useful as daily or frequent use products including but not limited to shampoos, conditioners, gels, mousses, pomades, anti-frizz agents, sprays, or hair dyeing products that may be applied to the hair as part of customary hair care procedures including washing, conditioning, dyeing, drying, and styling. These compositions may be for use in a salon. In some embodiments, the peptide compositions (i.e., bond shaping formulations) may be suitable for home use.

The peptide compositions (i.e., bond shaping formulations) may be liquids, solids, or gels, and can be filled and stored in any suitable container, including bottles, cartons, tubes, and canisters.

The peptides disclosed herein can be made by expression in cells. The peptides can be produced in cells such as bacterial or yeast cells., Examples include, but are not limited to, E. coli and Saccharomyces cerevisiae; Standard methods of cell production are well known to one of skill in the art. Steps include:
1. cloning a sequence encoding the peptides into an expression plasmid;
2. introducing the recombinant expression plasmid into a cell;
3. expressing the peptide in the cell; and
4. isolating the peptide from the cell.

Alternatively, the peptide can be synthesized using chemical synthesis according to standard synthetic protocols.

The peptides disclosed herein can be used to shape a subject's hair as part of a hair formulation or hair care kit, particularly in a hair formulation or hair care kit for defining curls. The peptides disclosed herein can be applied, as part of a formulation, by hand, applicator bottle, applicator brush, dropper, spray bottle, or by any other suitable method and/or applicator. In some forms, the peptides disclosed herein can be in a hair formulation, optionally as part of a kit containing one or more additional formulations, and applied by hand, applicator bottle, applicator brush, dropper, spray bottle, or by any other suitable method and/or applicator. In these forms, the formulation containing the peptides, and the one or more additional formulations, can be applied sequentially in any desired order. Alternatively, the formulation containing the peptides can be mixed with one or more additional formulations, optionally the one or more additional formulations are provided in a kit with the formulation containing the peptide(s), and then applied to the hair as a mixture.

The subject could be any mammal, preferably a human.

The bond shaping formulations usually contain conventional cosmetically acceptable carriers and excipients which may be used, for example, to modify various properties of the composition and to improve aesthetics. Such cosmetically acceptable excipients are described above. Commonly used natural and synthetic excipients are described, for example, in International Cosmetic Ingredient Dictionary and Handbook, Twelfth Edition 2008, ISBN-10:1882621433, (hereinafter “Cosmetic Handbook”) and CTFA ingredient information (http://www.ctfa-online.org/pls/ctfa_online. home), the content of which is hereby incorporated by reference in its entirety.

Emulsifiers are typically used to help blend ingredients which otherwise would be immiscible. Emulsifiers may be synthetic or natural. Natural emulsifiers may include, but are not limited to, olive oil, olive oil/wheat protein, olive oil/oat protein, sucrose esters, rice bran emulsifiers and/or various other food and pharmaceutical grade emulsifiers, alone or in combination.

Synthetic emulsifiers may include, but are not limited to, silicone emulsifiers, such as dimethicone copolyols; sulfonates and sulfonic acid derivatives; phosphorous organic derivatives; sugar esters; fatty esters, such as sorbitan monolaurate, sorbitan stearate, sorbitan laurate, sorbitan palmitate, sorbitan oleate; polyesters/PEG (polyethylene glycol) derivatives, such as Polysorbate 20 (polyethylene glycol 20 sorbitan monolaurate); fatty acid esters of fatty alcohols, such as glyceryl stearate, isopropyl stearate, hexyl laurate; fatty acid amides; acyl lactylates; alkoxylated compounds, such as alkoxylated block polymers, alcohols, alkylphenols, amines, amides, fatty esters, fatty acids, oils, sugar esters and polyesters, fatty acid esters of fatty alcohols, and ethers of fatty alcohols; carboxylated alcohol ethoxylates and alkylphenol ethoxylates; carboxylic acides/fatty acids, and mixtures thereof. Other suitable emulsifiers can include, but are not limited to, copolymers of an unsaturated ester and styrene sulfonate monomer, dicetyl phosphate, cetearyl alcohol, glyceryl ester, polyglyceryl-6 oleate, polyoxyethylene glycol ether of cetearyl alcohol, stearic acid, polysorbate-20, ceteareth-20, lecithin, glycol stearate, polysorbate-60, polysorbate-80, sodium surfactin, or combinations thereof. More than one emulsifier may be included in the bond shaping formulation. Still other natural and synthetic emulsifiers may be found in the Cosmetic Handbook. The emulsifiers are optionally included in an amount ranging from about 0.01% to about 15% by weight of the formulation, about 0.01% to about 10% by weight of the formulation, or about 0.01% to about 1% by weight of the formulation.

Colorants are typically used to provide relatively uniform color to the final cosmetic composition. Colorants may be synthetic or natural. Natural colorants may include pigments or plant-derived colors. Natural pigments may be inorganic (mineral) or organic, white or non-white, and coated or uncoated particles. Natural colorants may include, for example, cerium oxide, chromium oxide, iron oxide, titanium dioxide, zinc oxide, zirconium oxide, carbon black, chromium, chromium hydroxide green, ferric blue, manganese violet, ultramarine blue, D&C and FD&C colors, azo, indigoid, insoluble metallic salts of certified color additives, referred to as the Lakes, and the like, and mixtures thereof.

Synthetic colorants may include, for example, triphenylmethane, anthraquinone, and xanthine dyes and mixtures thereof. Other natural and synthetic colorants may be found in the Cosmetic Handbook and CTFA ingredient information.

The bond shaping formulation may include one or more waxes. Waxes may be synthetic or natural. Natural waxes may include, for example, beeswax, carnauba wax, and/or candelilla wax, hydrogenated jojoba oil, hydrogenated jojoba wax, hydrogenated microcrystalline wax, hydrogenated rice bran wax. Synthetic waxes may include, for example, cetyl esters, montan acid wax, paraffin, PEG-6 beeswax, PEG-8 beeswax, sulfurized jojoba oil, synthetic beeswax, synthetic candelilla wax, synthetic carnauba wax, synthetic japan wax, synthetic jojoba oil, synthetic wax, stearoxy dimethicone, dimethicone behenate, stearyl dimethicone, and synthetic homo-and copolymer waxes from the ethylene series or mixtures thereof. Other natural and synthetic waxes and oils may be found in the Cosmetic Handbook and CTFA ingredient information.

The bond shaping formulation also may include one or more preservatives. Preservatives may be either synthetic or natural and may be used to inhibit growth of undesirable microorganisms. Natural preservatives may include black currant fruit extract, aspen bark, radish root, and sorbic acid, alone or in combination.

Synthetic preservatives may include, for example, methylparaben, ethylparaben, propylparaben, imidazolidinyl urea, diazolidinyl urea, DMDM hydantoin, isothiazolinones, chlorinated aromatic compounds, para-hydroxybenzoic acids/parabens, alone or in combination. Other suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), propanediol, sodium benzoate, ethylenediamine-tetraacetic acid (EDTA), behentrimonium chloride, potassium sorbate, and/or grapefruit seed extract, or combinations thereof. More than one preservative may be included in the compositions. Other preservatives are known in the cosmetics industries and include salicylic acid, DMDM Hydantoin, Formaldahyde, Chlorphenesin, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, and Vitamin E (tocopherol). Still other natural and synthetic preservatives may be found in the Cosmetic Handbook and CTFA ingredient information. In some instances, the preservative(s) are optionally included in an amount ranging from about 0.1% to about 5% by weight of the bond shaping formulation or from about 0.3% to about 3% by weight of the bond shaping formulation. Preferably, the bond shaping formulations disclosed are paraben free.

The bond shaping formulation also may include one or more thickeners. Thickeners may either be synthetic or natural. Thickeners may be used to gel or thicken cosmetic compositions to provide, for example, better deposition properties. Natural thickeners may include waxes, gums and powders and mixtures thereof. Natural waxes may include beeswax, carnauba, and/or candelilla and mixtures thereof. Natural gums may include acacia, xanthan, schelortium (amigel), and/or cellulose and mixtures thereof. Natural powders may include clay, diatomaceous earth, fuller's earth, silica, silica shells or spherical silica, fumed silica, spherical silica, hydrated silica, silica silylate, mica, titanated mica, talc, cellulose or spherical cellulose beads, microcrystalline cellulose, corn starch, rice starch, glyceryl starch, soy flour, walnut shell powder, agar, sericite, dextran, nylon, silk powder, chalk, calcium carbonate, bismuth oxychloride, iron oxide, titanium dioxide, aluminum silicate, magnesium aluminum silicate, calcium silicate, magnesium trisilicate, aluminum starch octenylsuccinate, bentonite, hectorite, kaolin, maltodextrin, montmorillonite, zinc laurate, zinc myristate, zinc rosinate, alumina, attapulgite, tin oxide, titanium hydroxide, trimagnesium phosphate, or mixtures thereof.

Synthetic thickeners may include, for example, AMP isostearoyl hydrolyzed collagen, AMP isostearoyl hydrolyzed wheat protein, cetyl hydroxyethylcellulose, chondroitin sulfate, cocoamidopropyldimethylamine C8-16 isoalkysuccinyl lactoglobulin sulfonate, cocodimonium hydroxypropyl hydrolyzed collagen, distarch phosphate, ethyl ester of hydrolyzed animal protein, guar hydroxypropyltrimonium chloride, hydrolyzed animal or plant protein, hydroxypropyl guar, hydroxypropylmethyl cellulose, hydroxypropyl cellulose, hydroxyethyl cellulose, isostearoyl hydrolyzed collagen, methylcellulose, nitrocellulose, nonoxynyl hydroxyethylcellulose, acrylate polymers, acrylamine polymers, acrylic acid polymers (carbomer), PVM/MA Decadiene crosspolymers, polyvinylpyrrolidone polymers, silicone oils, polyethylene thickeners, aluminum starch octenyl succinate, trihydroxystearin, and mixtures thereof. Other natural and synthetic thickeners may be found in the Cosmetic Handbook and CTFA ingredient information.

The bond shaping formulation may include one or more conditioning agents or treatment agents. Conditioning or treatment agents may be either synthetic or natural. Natural conditioning or treatment agents may include panthenol, vitamins, lauroyl lysine, isostearic acid, lecithin, soft emollient waxes, fruit/botantical complexes, sweet almond oil, coconut oil, jojoba blends, honey, seaweed/algae, aloe, acai extract, wild pansy extract, lotus extract, papaya extract, zuzu extract, and/or orchid extract. Other suitable conditioning agents can include, but are not limited to, silicone-based agents (e.g., silicone quaternium-8), hydrolyzed wheat and/or soy protein, amino acids (e.g. wheat amino acids, arginine), rice bran wax, meadowfoam seed oil, mango seed oil, grape seed oil, jojoba seed oil, sweet almond oil, hydroxyethyl behenamidopropyl dimonium chloride, aloe leaf extract, aloe barbadensis leaf juice, phytantriol, retinyl palmitate, behentrimonium methosulfate, cyclopentasiloxane, quaternium-91, stearamidopropyl dimethylamine, and combinations thereof. In some instances, the conditioning agent(s) are optionally included in an amount ranging from about 0.1% to about 5% by weight of the composition or from about 0.3% to about 3% by weight of the bond shaping formulation.

Synthetic conditioning and treatment agents may include, for example, mercaptyl-containing compounds, such as mercapto-containing quaternary nitrogen compounds; betaine/aliphatic organic acid; silicone oil, silicone gum, silicone polymers, fatty acids, esters of fatty acids, fatty alcohols, ethoxylates, polyol polyesters, glycerine, glycerin monoesters, glycerin polyesters, cholesterol esters, polyolefinic glycols, polyolefinic monoester, polyolefinic polyesters. Other natural and synthetic conditioning and treatment agents may be found in the Cosmetic Handbook and CTFA ingredient information.

The bond shaping formulation also may include one or more essential and natural oils. Essential oils may be synthetic or natural. Natural essential oils may include bergamot, chamomile german, chamomile maroc, chamomile roman, cinnamon zeylanicum, clove buds, eucalyptus globulus, frankincense, fennel, hyssop, juniper, lemon grass, mountain savory, niaouli, red thyme, rosemary, rose geranium, tagestes, and ylang ylang. Natural oils may include, for example, jojoba oil, sweet almond oil, coconut oil, shea butter, mango butter, and/or aloe vera butter or mixtures thereof.

Synthetic essential oils may include, for example, esters, such as acetylated castor oil, glyceryl stearate, glyceryl dioleate, glyceryl distearate, glyceryl trioctanoate, glyceryl distearate, glyceryl linoleate, glyceryl myristate, glyceryl isostearate, PEG castor oils, PEG glyceryl oleates, PEG glyceryl stearates, PEG glyceryl tallowates, PEG-4 diheptanoate, hydrogenated castor oil, isotridecyl isononanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, tridecyl octanoate, octyl palmitate, dioctyl malate, tridecyl octanoate, myristyl myristate, octododecanol; fatty alcohols such as oleyl alcohol, isocetyl alcohol; and also silicone oils, isoparaffins, hydrogenated polyisobutene, petrolatum, lanolin derivatives, and sorbitan derivatives. Other natural and synthetic oils may be found in the Cosmetic Handbook and CTFA ingredient information.

The bond shaping formulation also may include one or more herbs and/or extracts and/or filtrates of herbs, such as, for example, Acacia Catechu, Acanthopanax Gracilistylus, Cacsalpinia Sappan, Epimedium Spinosa, Paeonia lactiflora, Paeonia obovata, Atractylodes macrocephala, Glycyrrhiza uralexisis, Glycyrrhiza glabra, Lycium chinense, Nauclea rhyncholphylla, Cinnainomum cassia, Astragalus membranaceus, Scutellaria baicalensis, Schizonepeta tenuifolia, Ephedra sinica, Ophiopogon japonicus, Paeonia suffruticosa, Artemisia annua, Aretemisia apiacea, Panax notoginseng, Cornus officinalis, Acorius gramineus, Reluhania glutinosa, Gastrodia elata, Asparagus cochiichinensis, Cuscuta chinensis, Schizandra chinensis, Schizandra spenanthera, Magnolia liliflora, Epimedium brevicomum, Epimedium grandiflorun, Epimedium sagittatum, Houttuynia cordata, Polygala tenuifolia; and Perilla frutescens, and an Aloe Vera extract, alone or in any combination.

The bond shaping formulation also may include one or more plant seeds and/or extracts and/or filtrates of plant seeds obtained from a plant such as, for example, rapeseed (Brassica spp.), soybean (Glycine max), sunflower (Lianthus annuus), oil palm (Elaeis guineeis), cottonseed (Gossypium spp.), groundnut (Arachis hypogaea), coconut (Cocus nucifera), castor (Ricinus communis), safflower (Carthamus tinctorius), mustard (Brassica spp. and Sinapis alba), coriander, (Coriandrum sativum), squash (Cucurbita maxima), linseed/flax (Linum usitatissimum), Brazil nut (Bertholletia excelsa) jojoba (Simmondsia chinensis) and maize (Zea mays), alone or in combination.

The bond shaping formulation can also include one or more surfactants that are able to reduce the surface tension of water and cause the hair formulation to slip across or onto the skin or hair. Surfactants also include detergents and soap. The surfactants may be amphoteric, anionic, or cationic. Suitable surfactants that may be used in the formulation include, but are not limited to, caprylyl/capryl glucoside, sodium cocoyl glutamate, polyglyceryl-6 oleate, sodium surfactin, heptyl glucoside, 3-aminopropane sulfonic acid, almond amide, almond amidopropyl betaine, almond amidopropylamine oxide, aluminum hydrogenated tallow glutamate, aluminum lanolate, aminoethyl sulfate, aminopropyl lauryl glutamine, ammonium C12-15 alkyl sulfate, ammonium C12-15 pareth sulfate, ammonium C12-16 alkyl sulfate, ammonium C9-10 perfluoroalkylsulfonate, ammonium capryleth sulfate, ammonium capryleth-3 sulfate, ammonium monoglyceride sulfate, ammonium sulfate, ammonium isothionate, ammonium cocoyl sarcosinate, ammonium cumene sulfonate, ammonium dimethicone copolyol sulfate, ammonium dodecylbenzenesulfonate, ammonium isostearate, ammonium laureth sulfate, ammonium laureth-12 sulfate, ammonium laureth-5 sulfate, ammonium laureth-6 carboxylate, ammonium laureth-7 sulfate, ammonium laureth-8 carboxylate, ammonium laureth-9 sulfate, ammonium lauroyl sarcosinate, ammonium lauryl sulfate, ammonium lauryl sulfosuccinate, ammonium myreth sulfate, ammonium myristyl sulfate, ammonium nonoxynol-30 sulfate, ammonium nonoxynol-4 sulfate, ammonium oleate, ammonium palm kernel sulfate, ammonium polyacrylate, ammonium stearate, ammonium tallate, ammonium xylene sulfonate, ammonium xylene sulfonate, amp-isostearoyl gelatin/keratin amino acids/lysine hydroxypropyltrimonium chloride, amp-isostearoyl hydrolyzed collagen, apricot kernel oil PEG-6 esters, apricot amide, apricot amidopropyl betaine, arachideth-20, avocadamide, avocadamidopropyl betaine, babassuamide, babassuamidopropyl betaine, babassuamidopropylamine oxide, behenalkonium chloride, behenamide, behenamide, behenamidopropyl betaine, behenamine oxide, sodium laureth sulfate, sodium lauryl sulfate, a polyoxyether of lauryl alcohol or ceteareth-20, or combinations thereof. More than one surfactant may be included in the composition. The surfactants are optionally included in an amount ranging from about 0.1% to about 15% by weight of the formulation or about 1% to about 10% by weight of the bond shaping formulation. It is understood that certain surfactants, such as polyglyceryl-6 oleate and sodium surfactin, are also emulsifiers. When the surfactant(s) included in the formulation is/are emulsifiers, they are typically characterized as emulsifiers.

Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, ammonyx cetac, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-b-alanine, sodium N-lauryl-β-iminodipropionate, myristoamphoacetate, lauryl betaine and lauryl sulfobetaine.

The bond shaping formulation can also include one or more emollients that can protect against wetness or irritation, soften, soothe, coat, lubricate, moisturize, protect, and/or cleanse. Suitable emollients for use in the formulation can include, but are not limited to, isodecyl neopentanoate, a silicone compound (e.g., dimethicone, cyclomethicone, dimethicone copolyol or a mixture of cyclopentasiloxane and dimethicone/vinyldimethicone cross polymer, cyclopentasiloxane polysilicone), polyols such as sorbitol, glycerin, glyceryl caprylate, propylene glycol, ethylene glycol, polyethylene glycol, caprylyl glycol, polypropylene glycol, 1,3-butane diol, hexylene glycol, isoprene glycol, xylitol; ethylhexyl palmitate; a triglyceride such as caprylic/capric triglyceride and fatty acid ester such as cetearyl isononanoate or cetyl palmitate. In some embodiments, the emollient is dimethicone, amidodimethicone, dimethiconol, cyclopentasiloxane, potassium dimethicone PEG-7 panthenyl phosphate, cetyl stearyl alcohol, cetyl alcohol, cetearyl alcohol, glyceryl caprylate, or combinations thereof. More than one emollient may be included in the bond shaping formulation. The emollient can be optionally included in an amount ranging from about 0.5% to about 15% by weight of the formulation or from about 1% to about 10% by weight of the bond shaping formulation. It is understood that certain emollients can also fall under other categories of cosmetically acceptable ingredients. For example, glyceryl caprylate included in the formulation is considered an emollient, a surfactant, or a preservative, or a combination thereof, depending on the specific needs and other components in the formulation.

The bond shaping formulation can also include one or more diluents. Water is the preferred diluent. The bond shaping formulation typically contains greater than one percent (by weight) water, preferably greater than five percent (by weight) water, more preferably greater than 50% (by weight) water, and most preferably greater than 80% (by weight) water. Alcohols, such as ethyl alcohol isopropyl alcohol, may be used at low concentrations (about 0.5% by weight of the formulation) to enhance hair penetration and/or reduce odor.

The bond shaping formulation can also include one or more viscosity modifying agents, such as viscosity increasing agents. Classes of such agents include, but are not limited to, viscous liquids, such as polyethylene glycol, semisythetic polymers, such as semisynthetic cellulose derivatives, synthetic polymers, such as carbomers, poloxamers, and polyethyleneimines (e.g., PEI-10), naturally occurring polymers, such as acacia, tragacanth, alginates (e.g., sodium alginate), carrageenan, vegetable gums, such as xanthan gum, petroleum jelly, waxes, particulate associate colloids, such as bentonite, colloidal silicon dioxide, and microcrystalline cellulose, surfactants, such as PPG-2 hydroxyethyl coco/isostearamide, emulsifiers, such as disteareth-75 IPDI, and salts, such as sodium chloride, and combinations thereof.

The bond shaping formulation can also include one or more antioxidants. Examples include, but are not limited to, tocopheryls, BHT, ascorbic acid, camellia sinensis leaf extract, ascorbyl palmitate, magnesium ascorbyl phosphate, carotenoids, resveratrol, triethyl citrate, arbutin, kojic acid, tetrahexydecyl ascorbate, superoxide dismutase, zinc, sodium metabisulfite, lycopene, ubiquinone, and combinations thereof.

The bond shaping formulation can also include one or more opacifying agents. Opacifying agents are added to the formulations to make it opaque. Suitable opacifying agents include, but are not limited to, glycol distearate and ethoxylated fatty alcohols.

The bond shaping formulation may be in the form of a spray. The spray typically includes the peptides and a cosmetically acceptable carrier. In some embodiments, the carrier is water or a water and alcohol mixture. The spray formulation optionally includes an antioxidant, sunscreen agent, vitamin, protein, peptide, plant extract, humectant, oil, emollient, lubricant, thickener, hair conditioning agent, polymer, and/or surfactant. The spray formulation can include a preservative. In some instances, the spray includes a fragrance. In some embodiments, the spray includes a surfactant. The hair spray formulations may be dispensed from containers that include aerosol dispensers or pump spray dispensers. Such dispensers are known in the art and are commercially available from a variety of manufacturers. When the spray composition is dispensed from a pressurized aerosol container, a propellant may be used to force the formulation out of the container. Suitable propellants include, but are not limited to, a liquefiable gas or a halogenated propellant. Examples of suitable propellants include dimethyl ether and hydrocarbon propellants such as propane, n-butane, iso-butane, CFCs, and CFC-replacement propellants. The propellants may be used singly or admixed. The amount of propellant may range from about 10% to about 60% by weight of the spray composition. The propellant may be separated from the composition, as in a two-compartment container. Other suitable aerosol dispensers are those characterized by the propellant being compressed air, which can be filled into the dispenser using a pump or equivalent device prior to use. Conventional non-aerosol pump spray dispensers, i.e., atomizers, may also be used to apply the composition containing peptides to the hair.

In some instances, the bond shaping formulation may be in the form of a conditioner. The conditioner typically includes the peptides in a suitable carrier. Additionally, the conditioner may include one or more cationic polymers derived from polysaccharides, for example cationic cellulose derivatives, cationic starch derivatives, cationic guar derivatives (such as guar hydroxypropyltrimonium chloride), cationic locust bean gum derivatives, synthetic cationic polymers, polyquaternium polymers (such as polyquaternium-7, polyquaternium-11, polyquaternium-113), and mixtures or combinations of these agents. The conditioner may comprise one or more other synthetic or natural polymers or polymers derived from biological preparation processes, which are functionalized, where appropriate, for example with cationic or neutral groups. These polymers may have a stabilizing or strengthening action on the hair and/or a conditioning action (deposition on the surface of the hair).

In some instances, the bond shaping formulation may be in the form of a shampoo. The shampoo typically includes the peptides in a suitable carrier. The peptides may be included in any suitable concentration. Additionally, the shampoo may include from about 0.5% to about 20% by weight of a surfactant. Surfactants utilized in shampoo compositions are well-known in the art and are disclosed, for example, in U.S. Pat. No. 6,706,258 to Gallagher, et al. and U.S. Pat. No. 7,598,213 to Geary, et al.

In some cases, the bond shaping formulation may be in the form of a liquid formulation. In these instances, the liquid compositions may contain any suitable concentration of the peptides, as described above, in a suitable carrier, typically a diluent, such as described above.

In some forms, the bond shaping formulation contains Water/Aqua/Eau, Glycerin, Hydroxyethylcellulose, Oligopeptide-300 (Bond Shaping Technology™), Caprylyl Glycol, Trisodium Ethylenediamine Disuccinate, Citric Acid, Sodium Hydroxide, Chlorphenesin, Phenoxyethanol, Fragrance (Parfum).

### B. Exemplary Bond Building Formulation

In some forms, a bond building formulation is provided for treating curly hair. The bond building formulation contains an active agent (also referred to herein as “compound” or “binding agent”), capable of strengthening disulfide bonds. Strengthening disulfide bonds can involve repairing damaged hair disulfide bonds and/or rebuilding latent disulfide bonds in hair. Accordingly, in some forms, the bond building formulation can provide more defined and/or visibly healthier curls. In some forms, the bond building formulation can improve hair structure and/or hair strength.

The binding agents can be combined with one or more cosmetically acceptable carriers and/or excipients that are considered safe and effective to human hair, skin, and/or human scalp, and may be administered to an individual's hair without causing undesirable side effects, such as burning, itching, and/or redness, or similar adverse reactions. Exemplary cosmetically acceptable carriers and excipients that can be used in combination with the active agent in the bond building formulation include those as described above for the bond shaping formulations. For example, the bond building formulation contains the active agent in combination with water, stabilizer(s), hair fixer(s), conditioning agent(s), buffering agent(s), emulsifier(s), chelating agent(s), film forming agent(s), preservative(s), humectant(s), viscosity modifier(s), surfactant(s), fragrances, and colorant(s). In some forms, the bond building formulations may contain an excipient that renders the formulations neutral pH, or a pH ranging from about pH 3 to about pH 12, preferably from pH 5 to pH 8.

The binding agent is typically present in an amount ranging from about 0.01 wt % to about 50 wt % of the formulation, such as from about 0.01 wt % to about 25 wt %, from about 0.01 wt % to about 20 wt %, from about 0.01 wt % to about 15 wt %, from about 0.01 wt % to about 10 wt %, from about 0.01 wt % to about 5 wt %, from about 0.01 wt % to about 1 wt %, from about 0.01 wt % to about 0.5 wt % of the total weight of the bond building formulation. For example, the binding agent is about 0.01-0.1 wt % or about 0.01-0.05 wt % of the bond building formulation, such as about 0.03 wt % of the total weight of the bond building formulation.

The binding agent is stable in aqueous solution for a period of at least 2, 3, 4, 5, 6, 8, 9, 10, 11, or 12 months or longer at pH of 6 to 8 and a temperature of about 25-30° C., such as about 25° C. “Stable” as used herein with respect to shelf-life means that at least 40, 45, 50, 55, 60, 65, 70, 75, 80, 85, 90, or 95% of the reactive moieties are intact or to the extent that the reactive moieties react with water, the resulting product is also electrophilic.

### 1. Binding Agents

The binding agent contains at least two reactive moieties capable of reacting with a thiol. The binding agent optionally contains a linker between the two or more reactive moieties. The linker forms two or more ionic bonds with the reactive moieties. The reactive moieties, upon reaction with thiol groups on the hair follicle, form bonds that are stable, for example, hydrolytically stable. “Stable”, as used in reference to the bonds formed between thiol groups on hair follicles means the bonds remain intact for at least one week, two weeks, three weeks, four weeks, one month, or two months or longer when exposed to water at pH 6-8 at a temperature from about 5° C. to about 100° C., preferably from about 20° C. to about 75° C., more preferably from about 20°° C. to about 50° C., more preferably from about 25° C. to about 40° C., most preferably from about 25° C. to about 30° C. In some embodiments, the temperature is about 25° C. It is also preferred that the binding reaction occurs around room temperature, for example, from about 15° C. to about 35° C., preferably from about 20° C. to about 30° C., more preferably from about 22° C. to about 27° C.

The binding agents typically have a low molecular weight and are compatible with aqueous or solvent delivery systems. In some embodiments, the compound is water-soluble. The low molecular weight is preferred, as it allows the molecule to diffuse in and out of hair at a reasonable rate. Molecular weights of less than 10,000 Da, 8,000 Da, 6,000 Da, 5,000 Da, 4,000 Da, 3,000 Da, 2,000 Da, or 1,000 Da are preferred. In some embodiments, the molecular weight is less than 1500 Da, preferably less than 800 Da, most preferably less than 500 Daltons to achieve sufficient diffusion rates in conventional aqueous hair care systems.

i. Binding Agents Defined by Formula I In some embodiments, the binding agents have a structure according to Formula I:

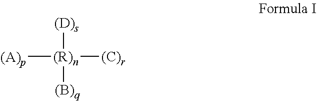

wherein

A, B, C, and D are reactive moieties containing one or more charges,

R is a linker that contains two or more charges, wherein the charges are opposite to the charges on the reactive moieties, n is an integer from 1 to 10, from 1 to 8, from 1 to 6, from 1 to 5, from 1 to 4, from 1 to 3, or 1 or 2; or an integer greater than 10, such as from >10 to 1000 or greater, from >10 to 1000, from >10 to 500, from >10 to 200, from >10 to 100, or from >10 to 50.

each occurrence of p, q, r, and s is independently an integer from 0 to 25, preferably from 0 to 10, more preferably from 0 to 2. The sum of p+q+r+s is equal to or greater than 2.

The reactive moieties may be present on any atom of the linker. In some embodiments, the reactive moieties are the same. In some embodiments, one or more of the reactive moieties is different.

In some embodiments, the reactive moieties are negatively charged and the linker or spacer has positively charged moieties. In other embodiments, the reactive moieties are positively charged and the linker or spacer has negatively charged moieties. Generally, the sum of the charges on the binding agent of Formula I is zero, though stoichiometric imbalances may exist.

ii. Linker

The reactive moieties on the binding agents are preferably linked via a linker. The term “linker”, as used herein, refers to one or more polyfunctional, e.g. bifunctional molecules, trifunctional molecules, tetrafunctional molecules, etc., which can be used to ionically bound the two or more reactive moieties and which do not interfere with the reactive properties of the binding agents. The reactive moieties may be attached to any part of the linker.

Linkers can be a single atom, such as a heteroatom (e.g., O or S), a group of atoms, such as a functional group (e.g., amine, —C(═O)—, —CH₂—), or multiple groups of atoms, such as an alkylene chain. Suitable linkers include but are not limited to oxygen, sulfur, carbon, boron, nitrogen, alkoxy, alkyl, alkenyl, cycloalkyl, cycloalkenyl, aryl, heterocycloalkyl, heteroaryl, ether, amine, and a polymer.

The linker is optionally independently substituted with one or more substituents including hydrogen, halogen, cyano, alkoxy, alkyl, alkenyl, cycloalkyl, cycloalkenyl, aryl, heterocycloalkyl, heteroaryl, amine, hydroxy, formyl, acyl, carboxylic acid (—COOH), —C(O)R¹, —C(O)OR¹, carboxylate (—COO⁻), primary amide (e.g., —CONH₂), secondary amide (e.g., —CONHR¹), —C(O)NR¹R², —NR¹R², —NR¹S(O)₂R², —NR¹C(O)R², —S(O)₂R², —SR¹, and —S(O)₂NR¹R², sulfinyl group (e.g., —SOR¹), and sulfonyl group (e.g., —SOOR¹); wherein R¹ and R² may each independently be hydrogen, alkyl, alkenyl, alkynyl, cycloalkyl, aryl, heterocycloalkyl and heteroaryl; wherein each of R¹ and R² is optionally independently substituted with one or more substituents selected from the group consisting of halogen, hydroxyl, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxyl, cycloalkyl optionally substituted with hydroxyl, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl.

In some embodiments, the linker may be an alkoxy, ether, alkyl, alkenyl, cycloalkyl, cycloalkenyl, aryl, heterocycloalkyl, heteroaryl, amine, or a polymer. In some embodiments, the linker is not a polymer, and n is from 1 to 10, from 1 to 8, from 1 to 6, from 1 to 5, from 1 to 4, from 1 to 3, or 1 or 2, such as 1, 2, 3, 4, 5, 6, 7, 8, 9, or 10. In some embodiments, the linker may be an oligomer, and n is from 3 to 10, from 3 to 8, from 3 to 6, from 3 to 5, or 3 or 4, such as 3, 4, 5, 6, 7, 8, 9, or 10.

iii. Polymeric Binding Agents

The binding agent can be a polymer. In this form, the linker forms or is the polymer backbone having ionically associated therewith two or more reactive moieties. Optionally, the polymeric binding agent can have a structure according to Formula I. In some forms, for each occurrence of a monomer unit in the polymer, zero, one, two, three, four, or more reactive moieties can be ionically associated with, the monomer. The reactive moieties on each monomer unit in the polymer can be the same or different.

In some embodiments, at least one reactive moiety is present on each monomer unit. Alternately, the reactive moieties may be present on alternate monomer units. In some embodiments, reactive moieties are present on a minimum percentage of the monomer units in the polymer. For example, at least one reactive moiety can be present on 0.1%, 1%, 5%, 10%, 15%, 20%, 25%, 30%, 35%, 40%, 45%, 50%, 55%, 60%, 65%, 70%, 75%, 80%, 85%, 90%, 95%, or 100% of the monomer units in the polymer. The reactive moieties can be present on any atom on the monomer.

a. Polymers

The polymer may be functionalized at the termini (and/or within the polymer backbone) with one or more of reactive moieties, A-D. One or more monomers in the polymer may be functionalized so that one or more reactive moieties, A-D, may be introduced (e.g., ionically associated with) using techniques known in the art. For ionically associated moieties, the salt is typically generated in situ.

A wide variety of polymers and methods for forming the polymers are known in the art of polymer science. Polymers can be degradable or non-degradable polymers. Polymers can be natural or unnatural (synthetic) polymers. Polymers can be homopolymers or copolymers comprising two or more monomers. In terms of sequence, copolymers can be random, block, or comprise a combination of random and block sequences. The polymers can in some embodiments be linear polymers, branched polymers, or hyperbranched/dendritic polymers. The polymers may also be present as a bound particle or surface functionalized inorganic particle. Suitable polymers include, but are not limited to poly (vinyl acetate), copolymers of styrene and alkyl acrylates, and copolymers of vinyl acetate and acrylic acid, polyvinylpyrrolidone, dextran, carboxymethylcellulose, polyethylene glycol, polyalkylene, polyacrylates, and polymethacrylates; polyanhydrides; polyorthoesters; polystyrene (PS), poly(ethylene-co-maleic anhydride), poly(ethylene maleic anhydride-co-L-dopamine), poly(ethylene maleic anhydride-co-phenylalanine), poly(ethylene maleic anhydride-co-tyrosine), poly(butadiene-co-maleic anhydride), poly(butadiene maleic anhydride-co-L-dopamine) (pBMAD), poly(butadiene maleic anhydride-co-phenylalanine), poly(butadiene maleic anhydride-co-tyrosine), poly(bis carboxy phenoxy propane-co-sebacic anhydride) (poly (CCP:SA)), alginate; and poly (fumaric anhydride-co-sebacic anhydride (p[FA:SA]), copolymers of p[FA:SA], polyacrylates and polyacrylamides, and copolymers thereof, and combinations thereof. In some embodiments, the polymeric linker is preferably water-soluble.

For polymeric linkers, the number (n) of monomers is typically an integer greater than 10, such as from >10 to 1000 or greater, from >10 to 1000, from >10 to 500, from >10 to 200, from >10 to 100, or from >10 to 50.

iv. Reactive Moieties that React with Thiols

The binding agent contains at least two reactive moieties that react with thiols to form covalent bonds. The reactive moieties are capable of reacting with a thiol group in the hair or on the skin to form a stable covalent bond. The reactive moiety is typically an electrophilic moiety capable of forming a salt with the linker. Alternately, the reactive moiety can be a free radical forming moiety.

The binding agent contains at least two reactive moieties. However, the binding agent may contain three, four, five, six, or greater than six reactive moieties.

The reaction between the reactive moiety and the thiol groups may be initiated at room temperature and pressure when the reactive moiety contacts a thiol group in the hair or on the skin. In some embodiments, the reaction may require an initiator, such as heat, catalyst, basic conditions, or a free radical initiator. The rate of reaction between the reactive moiety and the thiol may be increased by changes in temperature, pH, and/or addition of one or more excipients, such as a catalyst; however, this is generally not required.

The two or more reactive moieties on the binding agent can be the same. In some embodiments, the two or more reactive moieties are different.

In some embodiments, the reactive moieties are capable of undergoing a conjugate additional reaction. The reactive moieties can independently be or contain a Michael acceptor, a succinimidyl-containing group, a maleimido-containing group, azlactone, a benzoxazinone derivative, vinyl sulfone, vinyl sulfoximine, vinyl sulfonate, vinyl phosphonate, benzoxazinone, isocyanate, epoxide, an electrophilic moiety containing a leaving group, an electrophilic thiol acceptor, acrylic or acrylate group, a methacrylic or methacrylate group, a styrene group, an acryl amide group, a methacryl amide group, a maleate group, a fumarate group, an itaconate group, a vinyl ether group, an allyl ether group, an allyl ester group, a vinyl ester group,a sulfonate group, a phosphonate group, a sulfoxide group, a sulfonamide group, a sulfinimide group, a sulfinamide group, a sulfonimidate group, or a sulfonimidamide group.

a. Michael Acceptor

A “Michael acceptor,” as used herein, is a compound or moiety with at least one Michael acceptor functional group with the structure below:

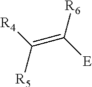

where E is —C(═O) R₃, —C(═O)OR₃, —C(═O)NHR₃, —CN, —S(O)R₃ or —S(O)₂R₃, where R₃, R₄, R₅, and R₆ taken independently, are hydrogen or a group or grouping selected from, but not limited to, alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, peptide, and polypeptide groups. In certain embodiments, R₃ and one of R₄, R₅, or R₆ may together form a ring.

Some suitable Michael acceptors include, but are not limited to molecules in which some or all of the structure above are residues of (meth) acrylic acid, fumaric acid, or maleic acid, substituted versions thereof, or combinations thereof, attached to the Michael acceptor molecule through an ester linkage.

The linker is attached to the Michael acceptor via R₃, R₄, R₅, or R₆. In some embodiments, R₃, R₄, R₅, or R₆ may be the linker.

b. Vinyl Sulfone

The chemistry of vinyl sulfones with respect to attack by nucleophiles is analogous to that of α,β-unsaturated ketones in that they can undergo a 1,4-type Michael addition without releasing any undesirable by-products.

c. Vinyl Sulfoximines

The chemistry of vinyl sulfoximines is similar to vinyl sulfones. The N-tosyl sulfoximine group is more electron withdrawing than the phenyl sulfone and therefore the vinyl groups will be more susceptible towards nucleophilic attack. N-substituents can be used to alter the electrophilic potential of the vinyl group.

d. Electrophilic Moiety Containing a Leaving Group

The reactive moiety may be an electrophile with a leaving group. Electrophile, as used herein refers to one or more functional groups or moieties that have an affinity for or attract electrons. Suitable electrophiles include, but are not limited to, ester moieties (—(CO)—O—R, wherein R is lower alkyl or the like), carbonyl moieties (—C(O)), carboxylic acid or carbonic acid (—COOH or —OCOOH), carbonate moieties (—O—(CO)—O—R, wherein R is lower alkyl or the like), urethane moieties (—O—(CO)—NH—R, wherein R is H, lower alkyl, or the like), substituted urethane moieties (—O—(CO)—NR′—R, where R′ is a nonhydrogen substituent such as alkyl, aryl, alkaryl, or the like), amido moieties (—(CO)—NH—R, wherein R is H, lower alkyl, or the like), substituted amido moieties (—(CO)—NR′—R where R′ is as defined previously), thioester moieties (—(CO)—S—R, wherein R is H, lower alkyl, or the like), sulfonic ester moieties (—S(O)₂—O—R, wherein R is H, lower alkyl, or the like), and the like. Other electrophiles will be known to those of ordinary skill in the art of organic chemistry and polymer science and/or can be readily found by reference to the pertinent texts and literature.

The electrophiles preferably contain a leaving group. Suitable leaving groups are well known in the art, see, e.g., “Advanced Organic Chemistry,” Jerry March, 5th Ed., pp. 445-448, John Wiley and Sons, N.Y. Examples of leaving groups include, but are not limited to, halogen, sulfonyloxy, optionally substituted alkylsulfonyloxy, optionally substituted alkenylsulfonyloxy, optionally substituted arylsulfonyloxy. Specific examples of leaving groups include chloro, iodo, bromo, fluoro, methanesulfonyloxy (mesyloxy), tosyloxy, triflyloxy, nitrophenylsulfonyloxy (nosyloxy), bromophenylsulfonyloxy (brosyloxy), hydroxyl, carboxylate, carbonate, phosphate, phosphonate, phosphinate, phosphonium, urethane, urea, amide, imide, amine, ammonium, sulfonato, —N₃, CN, RO—, NH₂O—, NHRO—, N(R⁴)₂O—, R⁴CO₂—, R⁴OCO₂—, R⁴NCO₂—, R⁴S—, R⁴C(S)O—, R⁴CS₂—, R⁴SC(O)S—, R⁴SCS₂—R⁴SCO₂—, R⁴OC(S)O—, R⁴OCS₂—, R⁴SO₂—, R⁴SO₃—, R⁴OSO₂—, R⁴OSO₃—, R⁴PO₃—, R⁴OPO₃—, an N-imidazolyl group, an N-triazolyl group, an N-benzotriazolyl group, a benzotriazolyloxy group, an imidazolyloxy group, an N-imidazolinone group, an N-imidazolone group, an N-imidazolinethione group, an N-imidazolinethione group, an N-succinimidyl group, an N-phthalimidyl group, an N-succinimidyloxy group, an N-phthalimidyloxy group, —ON═C(CN)R⁴, and a 2-pyridyloxy group. R⁴ is preferably an alkyl group or an aryl group.

Preferably, the leaving group is removed from the reactive moieties and does not result in the formation of side product that disadvantageously affects the reaction between the reactive moieties and the thiol groups or form a material or compound that is unsuitable for contact with skin or hair.

In some embodiments, the leaving group is a halogen.

e. Electrophilic Thiol Acceptors

Electrophilic thiol acceptors, as used herein, refer to a chemical moiety that reacts with a thiol group so that the sulfur atom of the thiol group becomes covalently bonded to the thiol acceptor. Thiol acceptors are well known in the art. Koval (Reactions of Thiols, Russian Journal of Organic Chemistry, 2007, 43:319-349) discloses several electrophilic thiol acceptors, the disclosure of which is incorporated herein by reference.

Electrophilic thiol acceptors, in addition to those listed above, include but are not limited to an alpha-substituted acetyl group with the formula Y—CH₂—CO— wherein Y is a leaving group. Examples of leaving groups include, but are not limited to, chloride, bromide, iodide, mesylate, tosylate, and the like. If the thiol acceptor is an alpha-substituted acetyl group, the thiol adduct after covalent linkage to the acceptor forms the bond —S—CH₂—.

f. Free Radical-Forming Groups

The binding agent may contain at least two free radical-forming groups that can react with thiols. The free radical-forming groups on the binding agent can be the same. Alternately, the free radical-forming groups may be different. Suitable free radical forming groups include, but are not limited to acrylate groups, methacrylate groups, styrene groups, acryl amide groups, methacryl amide groups, maleate groups, fumarate groups, itaconate groups, vinyl ether groups, allyl ether groups, allyl ester groups, and vinyl ester groups. For example, suitable binding agents include ethylene glycol dimethacrylate, diethylene glycol diacrylate, allyl methacrylate, trimethylolpropane triacrylate, triallylamine, tetraallyloxyethane, and di-and triacrylates, mixed acrylates which, as well as acrylate groups, comprise further ethylenically unsaturated groups. Other examples of binding agents include N, N′-methylenebisacrylamide and N, N′-methylenebismethacrylamide, esters of unsaturated mono-or polycarboxylic acids of polyols, such as diacrylate or triacrylate, for example butanediol diacrylate, butanediol dimethacrylate, ethylene glycol diacrylate, ethylene glycol dimethacrylate and also trimethylolpropane triacrylate and allyl compounds, such as allyl (meth) acrylate, triallyl cyanurate, diallyl maleate, polyallyl esters, tetraallyloxyethane, triallylamine, tetraallylethylenediamine, allyl esters of phosphoric acid and also vinylphosphonic acid derivatives, pentaerythritol diallyl ether, pentaerythritol triallyl ether, pentaerythritol tetraallyl ether, polyethylene glycol diallyl ether, ethylene glycol diallyl ether, glycerol diallyl ether, glycerol triallyl ether, polyallyl ethers based on sorbitol, and also ethoxylated variants thereof. Other examples of binding agents include di-and triacrylates of 3- to 15-tuply ethoxylated glycerol, of 3- to 15-tuply ethoxylated trimethylolpropane, of 3- to 15-tuply ethoxylated trimethylolethane, especially di-and triacrylates of 2- to 6-tuply ethoxylated glycerol or of 2- to 6-tuply ethoxylated trimethylolpropane, of 3-tuply propoxylated glycerol, of 3-tuply propoxylated trimethylolpropane, and also of 3-tuply mixed ethoxylated or propoxylated glycerol, of 3-tuply mixed ethoxylated or propoxylated trimethylolpropane, of 15-tuply ethoxylated glycerol, of 15-tuply ethoxylated trimethylolpropane, of 40-tuply ethoxylated glycerol, of 40-tuply ethoxylated trimethylolethane and also of 40-tuply ethoxylated trimethylolpropane, ethylene glycol dimethacrylate, diethylene glycol diacrylate, allyl methacrylate, trimethylolpropane triacrylate, triallylamine, tetraallyloxyethane, N,N′-methylenebisacrylamide, N,N′-methylenebismethacrylamide, butanediol diacrylate, butanediol dimethacrylate, trimethylolpropane triacrylate, triallyl cyanurate, diallyl maleate, a polyallyl ester, tetraallylethylenediamine, pentaerythritol diallyl ether, pentaerthyritol triallyl ether, pentaerythritol tetraallyl ether, polyethylene glycol diallyl ether, ethylene glycol diallyl ether, glycerol diallyl ether, glycerol triallyl ether, di-and triacrylates of 3- to 15-tuply ethoxylated glycerol, di- and tri-acrylates of 3- to 15-tuply ethoxylated trimethylolpropane, and di- and tri-acrylates of 3- to 15-tuply ethoxylated trimethylolethane. As used herein, the term “tuply” refers to the number of monomeric units in the ethoxylated chain.

The reactive free radical moieties may require the presence of one or more initiators. Suitable initiators include, but are not limited to peroxides, hydroperoxides, hydrogen peroxide, persulfates, azo compounds, and redox initiators. Suitable organic peroxides include acetylacetone peroxide, methyl ethyl ketone peroxide, tert-butyl hydroperoxide, cumene hydroperoxide, tert-amyl perpivalate, tert-butyl perpivalate, tert-butyl perneohexanoate, tert-butyl perisobutyrate, tert-butyl per-2-ethylhexanoate, tert-butyl perisononanoate, tert-butyl permaleate, tert-butyl perbenzoate, di(2-ethylhexyl) peroxydicarbonate, dicyclohexyl peroxydicarbonate, di(4-tert-butylcyclohexyl) peroxydicarbonate, dimyristil peroxydicarbonate, diacetyl peroxydicarbonate, allyl peresters, cumyl peroxyneodecanoate, tert-butyl per-3,5,5-trimethylhexanoate, acetylcyclohexylsulfonyl peroxide, dilauryl peroxide, dibenzoyl peroxide, and tert-aryl perneodecanoate. Suitable azo compounds include 2,2′-azobisisobutyronitrile, 2,2′-azobis(2,4-dimethylvaleronitrile) and 2,2′-azobis(4-methoxy-2,4-dimethylvaleronitrile), preferably water-soluble azo initiators, such as, but not limited to, 2,2′-azobis{2-[1-(2-hydroxyethyl)-2-imidazolin-2-yl]propane}dihydrochloride, 2,2′-azobis-(2-amidinopropane) dihydrochloride, 2,2′-azobis[2-(2-imidazolin-2-yl)propane]dihydrochloride and 2,2′-azobis[2-(5-methyl-2-imidazolin-2-yl)propane]dihydrochloride. For the redox initiators, the oxidizing component is at least one of the peroxo compounds indicated above and the reducing component is, for example, ascorbic acid, glucose, sorbose, ammonium bisulfite, ammonium sulfite, ammonium thiosulfate, ammonium hyposulfite, ammonium pyrosulfite, ammonium sulfide, alkali metal bisulfite, alkali metal sulfite, alkali metal thiosulfate, alkali metal hyposulfite, alkali metal pyrosulfite, alkali metal sulfide, or sodium hydroxymethylsulfoxylate.

In some embodiments, the binding agent has the structure:

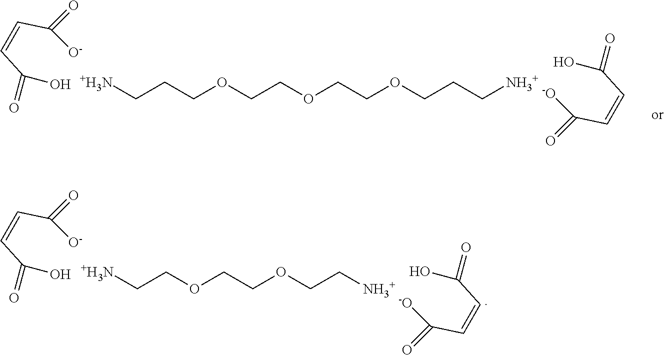

In some embodiments, the binding agent is bis-aminopropyl diglycol dimaleate.

Other binding agents include, but are not limited to, acid-containing electrophiles, such as acrylic acid and bromo-acetic acid and similar compounds.

### 2. Cosmetically Acceptable Carrier and Excipients

The bonding building formulations typically contain one or more cosmetically acceptable carrier, such as water, and one or more cosmetically acceptable excipients. Cosmetically acceptable carriers and excipients that can be used in combination with the active agent in the bond building formulation include those as described above for the bond shaping formulations. For example, cosmetically acceptable excipients suitable for use in the bond building formulations include, but are not limited to, preservatives, antioxidants, chelating agents, sunscreen agents, vitamins, dyes, hair coloring agents, proteins, amino acids, natural extracts such as plant extracts, humectants, fragrances, perfumes, oils, emollients, lubricants, butters, penetrants, thickeners, viscosity modifiers, polymers, resins, hair fixatives, film formers, surfactants, detergents, emulsifiers, opacifying agents, volatiles, propellants, liquid vehicles, carriers, salts, pH adjusting agents (e.g., citric acid), neutralizing agents, buffers, hair conditioning agents, anti-static agents, anti-frizz agents, anti-dandruff agents, and absorbents, and combinations thereof.

The bond building formulations can contain the binding agent, water, and optionally at least two or more cosmetically acceptable excipients. In some forms, the bond building formulations contain the binding agent, water, and optionally stabilizer(s), hair fixer(s), conditioning agent(s), buffering agent(s), emulsifier(s), chelating agent(s), film forming agent(s), preservative(s), humectant(s), viscosity modifier(s), surfactant(s), fragrances, and/or colorant(s). In some forms, the bond building formulations contain the binding agent, water, and optionally a preservative and/or fragrance. In some forms, the bond building formulations contain the binding agent, water, and optionally: a blend of humidity-resistant, flexible polymers, plant collagen, and sugars that form a curl-locking barrier to maintain definition, smoothness and shine; and/or a blend of lauric acid, fatty acids, and essential vitamins to help fortify hair and reduce breakage while providing nourishment and conditioning for softer, shinier-looking hair.

The cosmetically acceptable carrier(s) and excipient(s) are typically present in an amount ranging from about 10 wt % to about 99.99 wt % of the formulation, preferably about 40 wt % to about 99 wt %, more preferably from about 80 wt % to about to about 99 wt %. More specific exemplary bond building formulations are described in the Examples below.

### 3. Forms of the Bond Building Formulation

The bond building formulation may be in any suitable physical form. Suitable forms include, but are not limited to, low to moderate viscosity liquids, lotions, milks, mousses, sprays, gels, creams, shampoos, conditioners, and the like. In some forms, the bond building formulation is a leave-in formulation in a suitable form, such as a spray or conditioner. When provided as a leave-in formulation (i.e., a no-rinse formulation), the bond building formulation can be applied to hair, and the hair is not rinsed or washed for at least 24 hours, at least 36 hours, at least 48 hours, at least 60 hours, or at least 72 hours. Suitable excipients, such as those listed above, are included or excluded from the hair care formulation depending on the form of use of the formulation (e.g., hair spray, cream, conditioner, or shampoo).

i. Sprays

The bond building formulation may be in the form of a spray. The spray typically includes the binding agent and a cosmetically acceptable carrier. In some embodiments, the carrier is water or a water and alcohol mixture. The spray formulation optionally includes an antioxidant, sunscreen agent, vitamin, protein, peptide, plant extract, humectant, oil, emollient, lubricant, thickener, hair conditioning agent, polymer, and/or surfactant. Preferably, the spray formulation includes a preservative. In some embodiments, the formulation includes a fragrance. In some embodiments, the formulation includes a surfactant. In some embodiments, the formulation contains water, fragrance, a preservative, and a binding agent. In some embodiments, the formulation contains water, fragrance, a preservative, and a binding agent. In some embodiments, the formulation contains water, a preservative, fragrance, the binding agent, and an anti-static agent. In some embodiments, the formulation contains water, a preservative, fragrance, the binding agent, and a hair conditioning agent. In some embodiments, the formulation contains water, a preservative, fragrance, the binding agent, and a surfactant.

When in the form of a spray, the bond building formulations may be dispensed from containers that include aerosol dispensers or pump spray dispensers. Such dispensers are known in the art and are commercially available from a variety of manufacturers.

a. Propellant

When the bond building spray formulation is dispensed from a pressurized aerosol container, a propellant may be used to force the composition out of the container. Suitable propellants include, but are not limited to, a liquefiable gas or a halogenated propellant. Examples of suitable propellants include dimethyl ether and hydrocarbon propellants such as propane, n-butane, iso-butane, CFCs, and CFC-replacement propellants. The propellants may be used singly or admixed.

The amount of propellant may range from about 10% to about 60% by weight of the formulation. The propellant may be separated from the hair repair formulation as in a two compartment container. Other suitable aerosol dispensers are those characterized by the propellant being compressed air, which can be filled into the dispenser using a pump or equivalent device prior to use. Conventional non-aerosol pump spray dispensers, i.e., atomizers, may also be used to apply the hair strengthening formulation to the hair.

ii. Conditioners

The bond building formulation may be in the form of a conditioner. The conditioner typically includes the binding agent in a suitable carrier. Additionally, the conditioner may include cationic polymers derived from polysaccharides, for example cationic cellulose derivatives, cationic starch derivatives, cationic guar derivatives and cationic locust bean gum derivatives, synthetic cationic polymers, mixtures or combinations of these agents. The formulation may comprise other synthetic or natural polymers or polymers derived from biological preparation processes, which are functionalized, where appropriate, for example with cationic or neutral groups. These polymers may have a stabilizing or strengthening action on the compositions, and/or a conditioning action (deposition on the surface of the skin or the hair).

The binding agent may be included in any suitable concentration. Typical concentrations of the binding agent in the conditioner range from small amounts such as approximately 0.01% (wt), preferably at least 0.1% (wt), to large amounts, such as up to 50% (wt). Preferably the conditioner contains the binding agent in a concentration ranging from 0.1% (wt) to 5% (wt), more preferably from 0.1% wt to 3% (wt). While greater concentrations of binding agent could be present in the conditioner, they are generally not needed to achieve the desired results.

iii. Shampoos

The bond building formulation may be in the form of a shampoo. The shampoo typically includes the binding agent in a suitable carrier. The binding agent may be included in any suitable concentration. Typical concentrations of the binding agent in the shampoo range from small amounts such as approximately 0.01% (wt), at least 0.1% (wt), to large amounts, such as up to 10%, 20%, 30%, 40%, or 50% (wt). Preferably the shampoo contains the binding agent in a concentration ranging from 0.1% (wt) to 5% (wt), more preferably from 0.1% wt to 3% (wt). While greater concentrations of binding agent could be present in the shampoo, they are generally not needed to achieve the desired results.

Additionally, the shampoo may include from about 0.5% to about 20% of a surfactant material. Surfactants utilized in shampoo compositions are well-known in the art and are disclosed, for example, in U.S. Pat. No. 6,706,258 to Gallagher et al. and U.S. Pat. No. 7,598,213 to Geary et al.

iv. Creams

The bond building formulation may be in the form of a cream. The cream typically includes the binding agent in a suitable carrier. The binding agent may be included in any suitable concentration. Typical concentrations of the binding agent in the cream range from small amounts such as approximately 0.01% (wt), preferably at least 0.1% (wt), to large amounts, such as up to 50% (wt). Preferably the cream contains the binding agent in a concentration ranging from 0.1% (wt) to 5% (wt), more preferably from 0.1% wt to 3% (wt). While greater concentrations of binding agent could be present in the cream, they are generally not needed to achieve the desired results.

Additionally, the cream may include an oil, a hair conditioning agent, and/or a thickening agent. The cream may also include a fragrance, a plant extract, and/or a surfactant. The cream may be packaged in a tube, tub, bottle, or other suitable container.

v. Liquid Bond Building Formulations

In some embodiments, a liquid bond building formulation is provided. The liquid bond building formulation may be applied by itself to the hair, either as a stand-alone treatment or a step in a treatment involving multiple steps; or is mixed at the time of use with another formulation, such as the bond shaping formulation or an enhancing formulation, and then applied to the hair. In these embodiments, the liquid bond building formulation may contain any suitable concentration of binding agent in a suitable carrier, typically a diluent, such as described above. If mixed with another formulation, the concentration of the binding agent in the bond building formulation is suitable to provide a mixture with the appropriate final volume and final concentration of binding agent.

For example, a liquid bond building formulation can contain a concentration of binding agent ranging from about 0.001% (wt) to about 0.1%, from about 0.01% to about 5% (wt), or from about 1% to about 3%, or greater amounts, such as from about 1% to about 50% (wt) or greater. In a preferred embodiment, the liquid bond building formulation contains about 20% (wt) binding agent.

In some forms, the bond building formulation contains Water/Aqua/Eau, Xylitylglucoside, Anhydroxylitol, Hydroxyethylcellulose, Bis-Aminopropyl Diglycol Dimaleate, Maltitol, Glycerin, Caprylyl/Capryl Glucoside, Xylitol, Sodium Polyacryloyldimethyl Taurate, PVM/MA Copolymer, Ethylhexylglycerin, Polyimide-1, Acacia Senegal Gum, Persea Gratissima (Avocado) Oil, Glycolipids, Cocos Nucifera (Coconut) Oil, Sodium Hydroxide, Sodium Cocoyl Glutamate, Olive Oil Polyglyceryl-6 Esters, Citric Acid, Glyceryl Stearate, Propanediol, Glyceryl Caprylate, Trisodium Ethylenediamine Disuccinate, Lactic Acid, Cetearyl Alcohol, Glyceryl Undecylenate, Phospholipids, Polyglyceryl-10 Dioleate, Polyglyceryl-10 Oleate, Cocos Nucifera (Coconut) Fruit Juice, Cocos Nucifera Water, Sodium Stearoyl Lactylate, Pelvetia Canaliculata Extract, Potassium Sorbate, Phenoxyethanol, Sodium Benzoate, Fragrance (Parfum), Linalool, Citronellol, Hexyl Cinnamal, Limonene, and Citral.

### C. Kits

The one or more formulations provided herein may be provided and/or used as part of a kit, such as a hair shaping kit for building/rebuilding curls. Typically, kits for shaping hair, particularly for shaping curly hair, contain a bond shaping formulation and a bond building formulation, as described herein. Alternatively, the bond shaping formulation and a bond building formulation can be provided separately for use in the same multi-step treatment.

The bond shaping formulation typically contains an effective amount of the peptide to replenish keratin while locking and shaping disulfide bonds into place for curl definition. The bond building formulation typically contains an effective amount of the binding agent to strengthen disulfide bonds that are responsible for hair structure and strength for visibly healthier curls. When provided as part of a kit and used in one treatment (applying the formulations sequentially or pre-mixing and then applying the mixture, to hair), such treatment can repair, strengthen, reshape, and visibly restore natural curl pattern, optionally after just one treatment. For example, after treatment using the kit, curls are shinier, bouncier, and more defined, optionally with results that last through multiple washes, with no heat required.

Each of the bond shaping formulation and bond building formulation in the kit may be in any suitable form. Suitable forms include, but are not limited to, low to moderate viscosity liquids, lotions, milks, mousses, sprays, gels, creams, shampoos, conditioners, and the like. Each of the bond shaping formulation and bond building formulation can be present in a suitable container, which depends on the form of the formulation. In some forms, ingredients in the bond shaping formulation and/or bond building formulation is provided in two or more separate containers or compartments. For example, the binding agent may be provided as a dry powder in a sealed package and the carrier/excipient provided in a vial or other container. A suitable mixing container for the binding agent and the carrier/excipient may be provided to form the bond building formulation. Similarly, the peptide may be provided as a dry powder in a sealed package and the carrier/excipient provided in a vial or other container. A suitable mixing container for the peptide and the carrier/excipient may be provided to form the bond shaping formulation.

Optionally, the bond shaping formulation and/or bond building formulation is premixed with a shampoo, a conditioner, or other formulation, such as an enhancing formulation. In some forms, the bond building formulation may be mixed with the bond shaping formulation or another formulation, such as an enhancing formulation, and the mixture is applied to the hair.

### 1. Enhancing Formulation

The kits may further include one or more additional formulations to facilitate application of the bond shaping formulation and/or bond building formulation, and/or to enhance the curl defining/building/rebuilding results. For example, the kit further includes an enhancing formulation containing one or more cosmetically acceptable carriers and one or more cosmetically acceptable excipients, such as those described above for the bond shaping and bond building formulations. Optionally, when cosmetically acceptable excipients are present in the enhancing formulation, the total amount of the cosmetically acceptable excipients is small (e.g., less than 5% by weight, less than 2% by weight, or less than 1% by weight of the formulation).

In some forms, an enhancing formulation can include water, an alcohol (such as propanediol), chelating agent(s), antimicrobial(s), surfactant(s), emollient(s), emulsifier(s), and fragrances. Alcohols, such as propanediol, may be used at low concentrations (e.g., less than 1% by weight of the formulation, for example, 0.5%-1% by weight of the formulation) in the enhancing formulation. Antimicrobials, such as hydrogen peroxide, may be used at a concentration of 0.1%-1% by weight in the enhancing formulation. Suitable chelating agents, surfactants, emollients, and emulsifiers for use in the enhancing formulation can be any of those described above for the bond shaping and bond building formulations. Optionally, each of the chelating agents, surfactants, emollients, and emulsifiers is present at low concentrations, such as less than 0.1% by weight or less than 0.05% by weight in the enhancing formulation.

Exemplary enhancing formulations are described in the Examples below.

In some forms, the enhancing formulation contains Water/Aqua/Eau, Propanediol, Caprylyl/Capryl Glucoside, Sodium Cocoyl Glutamate, Citric Acid, Trisodium Ethylenediamine Disuccinate, Polyglyceryl-6 Oleate, Glyceryl Caprylate, Sodium Surfactin, Sodium Hydroxide, Hydrogen Peroxide, Fragrance (Parfum), Hexyl Cinnamal, Limonene, and Citral.

The enhancing formulation may be provided as part of the kit for defining, building, and/or rebuilding curls or it may be provided separately from a kit containing the bond shaping and bond building formulations. In some forms, the enhancing formulation is a leave-in formulation (also referred to as a no-rinse formulation or a leave-on formulation) in a suitable form, such as a spray or conditioner. When provided as a leave-in formulation, the enhancing formulation can be applied to hair, and the hair is not rinsed or washed for at least 24 hours, at least 36 hours, at least 48 hours, at least 60 hours, or at least 72 hours.

Instructions for use of the formulations in the kit are also typically provided. The kit optionally contains a shampoo and/or conditioner. Suitable shampoos and conditioners include, but are not limited to Olaplex No. 4 Bond Maintenance® Shampoo, Olaplex No. 4C Bond Maintenance® Clarifying shampoo, Olaplex No. 5 Bond Maintenance® Conditioner, Olaplex No. 4P Blonde Enhancer™ Toning Shampoo, Olaplex No. 5P Blonde Enhancer™ Toning Conditioner, etc. The kit may further contain an odor eliminator. The odor eliminator can be incorporated into the reducing formulation. Alternately, the odor eliminator is present in a suitable container for use before or after washing the binding formulation from the hair. Some suitable odor eliminators are known to those of ordinary skill in the art. The kits may further include one or more tools for application of the formulations, such as bottle(s) (such as applicator bottles), gloves, comb, etc.

### D. Exemplary Methods of Use

The methods disclosed herein are concerned with treating curly hair to define, build, and/or rebuild curls. Typically, after one or more treatments with the bond shaping formulation and/or bond building formulation, preferably both, coils, curls and waves are visibly repaired, healthier (such as shinier and/or bouncier), more defined, and smoother. The curl defining, building, and/or rebuilding results are immediate and ongoing, optionally lasting for at least 15 washes following treatment. The hair treatment results can be evaluated by visual inspection and/or one or more tests described above, such as curl droop test, anti-breakage test, anti-frizz test, and/or wet combing test, such as described in Example 11. For example, after a single treatment with the bond shaping formulation and bond building formulation, and optionally the enhancing formulation, the curls of dry hair are shinier, bouncier, and/or more defined as compared to untreated hair from the same individual, based on visual inspection; the hair (after drying) has reduced frizz compared to untreated hair from the same individual or hair from the same individual that is washed with a non-conditioning shampoo, such as 15% SLES solution; the hair is stronger compared to untreated hair from the same individual or hair from the same individual that is washed with a non-conditioning shampoo, such as 15% SLES solution; and/or the hair has reduced breakage compared to untreated hair from the same individual or hair from the same individual that is washed with a non-conditioning shampoo, such as 15% SLES solution.

A typical curl defining, building, and/or rebuilding treatment generally includes two or three steps. In some forms, the curl defining, building and/or rebuilding treatment includes: (a) applying the bond shaping formulation to the hair; and (b) applying the bond building formulation to the hair. Optionally, the treatment further includes (c) applying the enhancing formulation to the hair.

The treatment steps (a)-(c) may be performed in any desired order or fashion. In some forms, the curl building/rebuilding treatment includes steps (a), (c), and (b) performed sequentially. In some forms, the bond shaping formulation and bond building formulation are mixed and then applied to the hair. In some forms, the bond shaping or bond building formulation is mixed with the enhancing formulation and then applied to the hair. For example, the bond shaping formulation is first applied to the hair; the bond building formulation is mixed with the enhancing formulation and then applied to the hair as a mixture.

Optionally, the curl defining, building, and/or rebuilding treatment further includes one or more additional steps, such as rinsing or washing the hair, drying the hair, and/or manually fixing the hair. When curl droop test and/or anti-frizz test are performed on hair after treatment, the hair is dried after the hair is treated with the bond building formulation, so that curls and/or fizziness of dry hair are evaluated.

### 1. Apply the Bond Shaping Formulation to the Hair

In some forms, in the curl building/rebuilding treatment, the bond shaping formulation that contains a peptide is applied to the hair as a first treatment step. The peptide in the bond shaping formulation is capable of penetrating deep within the cortex to help replenish keratin while locking and shaping disulfide bonds into place for enhanced curl definition.

Typically, the binding formulation is applied to clean hair. Thus, optionally, the hair is rinsed or washed prior to application of the bond shaping formulation. For example, the hair is shampooed and optionally conditioned prior to applying the bond shaping formulation. Alternately, the hair may only be rinsed with water prior to application of the bond shaping formulation.

The bond shaping formulation can be applied to the hair using any suitable method. For example, the bond shaping formulation is in a form that can be applied with an applicator bottle, such as a liquid form. Typically, the amount of bond shaping formulation applied is sufficient to saturate the hair. Optionally, following the application or final application (with repeated application) of the bond shaping formulation, the hair is manually worked to ensure the hair is fully saturated with the formulation, such as by raking through the hair in a downward motion to ensure the bond shaping formulation is fully saturated.

The bond shaping formulation may be applied to the hair as a single application, or application of the bond shaping formulation may be repeated one or more times. Typically, the amount of bond shaping formulation applied in each application is sufficient to saturate the hair or a section of the hair. In some embodiments, the volume of bond shaping formulation applied to the hair in each application is about 10 to about 250 mL, about 20 to about 200 mL, or about 50 to about 150 mL. The specific amount depends on hair length and density. In some embodiments, application of the bond shaping formulation could be repeated immediately (e.g. within 10 to 15 seconds) or approximately 1, 5, 7.5, 10, 12.5, 15, 17.5, or 20 minutes after the first application. In some embodiments, the second application is about 1 minute to about 10 minutes after the first application.

Typically, the bond shaping formulation is left on the hair for a suitable period of time, and preferably without rinsing between the first and the second treatment step. For example, the bond shaping formulation is left on the hair for about 1 min to about 20 minutes, from about 5 minutes to about 15 minutes, from about 7 minutes to about 10 minutes, after the application or final application of the bond shaping formulation. Following this treatment period, and without any rinse/wash step, the bond building formulation or the enhancing formulation, or a mixture of both, is applied to the hair that is already saturated with the bond shaping formulation.

Alternatively, the bond shaping formulation is rinsed from the hair after its application. The hair may be rinsed and washed immediately (e.g. within 10 to 15 seconds following application) after the application or final application (when the formulation is applied more than once) of the bond shaping formulation. Alternatively, the hair may be rinsed and washed about 5 minutes or later after the application or final application of the bond shaping formulation, such as about 5 minutes to about 30 minutes, preferably about 10 minutes after the application or final application of the bond shaping formulation to the hair.

### 2. Apply the Enhancing Formulation to the Hair

Optionally, subsequent to the treatment with the bond shaping formulation, an enhancing formulation is applied to the hair. The hair to which the bond shaping formulation is applied may be bond shaping formulation saturated hair (no rinse after treatment with the bond shaping formulation) or clean hair (the bond shaping formulation is removed from hair after treatment).

Alternatively, the enhancing formulation may be applied to the hair simultaneously with application of the bond shaping formulation, or mixed with the hair shaping formulation and then applied to the hair as a mixture. In some forms, the enhancing formulation may also be applied to the hair prior to application of the bond shaping formulation.

The enhancing formulation contains one or more cosmetically acceptable carriers and excipients, and can facilitate the application of the bond shaping formulation and/or bond building formulation, and/or enhance the curl building/rebuilding results.

The enhancing formulation can be applied to the hair using any suitable method. For example, the enhancing formulation is in a form that can be sprayed to the hair, such as a liquid form. Typically, the amount of enhancing formulation applied is sufficient to saturate the hair. Optionally, following the final application of the enhancing formulation, the hair is manually worked to ensure the hair is fully saturated with the formulation, such as by scrunching the hair into curls to ensure the enhancing formulation is properly worked through.

Typically, after the hair is saturated with the enhancing formulation, it is left on the hair for a suitable period of time, and preferably without rinse to continue the next treatment step. For example, the enhancing formulation is left on the hair for about 1 min to about 20 mins, from about 5 mins to about 15 mins, from about 7 mins to about 10 mins, after the final application of the enhancing formulation. Following this treatment period, without any rinse/wash, the bond building formulation is applied to the bond shaping formulation/enhancing formulation saturated hair.

Alternatively, the enhancing formulation is rinsed from the hair after its application. The hair may be rinsed and washed immediately (e.g. within 10 to 15 seconds following application) after the application or final application (when the formulation is applied more than once) of the enhancing formulation. Alternatively, the hair may be rinsed and washed about 5 minutes or later after the application or final application of the enhancing formulation, such as about 5 minutes to about 30 minutes, preferably about 10 minutes after the application or final application of the enhancing formulation to the hair.

### 3. Apply the Bond Building Formulation to the Hair

Subsequent to the treatment with the bond shaping formulation and optionally the enhancing formulation, the bond building formulation is applied to the hair. The binding agent in the bond building formulation is capable of strengthening the disulfide bonds responsible for structure and hair strength, thereby providing visibly healthier curls.

The hair to which the bond building formulation is applied may be bond shaping formulation saturated hair (no rinse after treatment with the bond shaping formulation), bond shaping formulation/enhancing formulation saturated hair (no rinse after treatment with the bond shaping formulation and enhancing formulation), or clean hair (the bond shaping formulation and enhancing formulation are removed from hair after treatment).

In some forms, the enhancing formulation may be applied to the hair simultaneously with application of the bond building formulation, or mixed with the bond building formulation and then applied to the hair as a mixture. For example, the enhancing formulation is applied to the hair as a mist intermittently during the application of the bond building formulation to re-wet the hair as needed.

The bond building formulation can be applied to the hair using any suitable method. For example, the bond building formulation is in a form that can be applied with an applicator bottle, such as a liquid form. Typically, the amount of bond building formulation applied is sufficient to saturate the hair. Optionally, following the application or final application (with repeated application) of the bond building formulation, the hair is manually worked to ensure the hair is fully saturated with the formulation, such as by raking through the hair in a downward motion and/or scrunching the curls to ensure the bond shaping formulation is fully saturated.

The bond building formulation may be applied to the hair as a single application, or application of the bond building formulation may be repeated one or more times. Typically, the amount of bond building formulation applied in each application is sufficient to saturate the hair or a section of the hair. In some embodiments, the volume of bond building formulation applied to the hair in each application is about 10 to about 200 mL or about 20 to about 100 mL. The specific amount depends on hair length and density. In some embodiments, application of the bond building formulation could be repeated immediately (e.g. within 10 to 15 seconds) or approximately 1, 5, 7.5, 10, 12.5, 15, 17.5, or 20 minutes after the first application. In some embodiments, the second application is about 1 minute to about 10 minutes after the first application.

Typically, the bond building formulation is left on the hair for a suitable period of time, such as from about 1 minute to about 20 minutes, from about 5 minutes to about 15 minutes, from about 7 minutes to about 10 minutes, after the application or final application of the bond building formulation. In some forms, the hair does not need to be washed or rinsed subsequent to application of the bond building formulation, and is optionally subject to drying (such as air dry or drying with a blow dryer) and/or manual fixation to define curls using a suitable curl defining technique (such as scrunching the curls, shaping the curls with a suitable tool, for example, rollers or a curler, etc.). The drying and/or curl defining steps can be performed immediately (e.g. within 60 seconds (one minute), two minutes, three minutes, four, or five minutes following application) or within about 30 mins following application of the application or final application of the bond building formulation. Optionally, the hair is not washed or rinsed subsequently to application of the bond building formulation for at least 24 hours, at least 36 hours, at least 48 hours, at least 60 hours, or at least 72 hours.

Alternatively, subsequent to application of the bond building formulation, the hair may be washed or rinsed to remove the formulations from the hair. For example, the hair may be rinsed and subsequently washed immediately (e.g. within 60 seconds (one minute), two minutes, three minutes, four, or five minutes following application) after the application or final application of the bond building formulation. Alternatively, the hair may be rinsed and washed within about 30 minutes following application, such as between about 5 minutes and about 20 minutes, or about 10 minutes after the application or final application of the bond building formulation to the hair, depending on the hair type. In these forms, the cleaned hair may be subject to drying (such as air dry or drying with a blow dryer) and/or curl-defining using a suitable technique (such as scrunching the curls, shaping the curls with a suitable tool, for example, a curler, etc.).

In some cases, step 2 and step 3 may be combined into a single step or step 2 and step 3 may occur simultaneously. In some other instances, fixation (step 3) may be performed after relaxation (step 1) and then application of the peptides (step 2) is performed. Each step can involve application of a solution to hair for about 5, 10, 15, 20, 25, or 30 minutes, though this time may range anywhere from approximately 5 minutes to 1 hour. In some instances, the hair relaxation composition is applied to the hair for a processing period ranging from about 10 to 15 minutes, 35 to 45 minutes, 45 to 60 minutes, or 25 to 35 minutes. In some cases, shorter processing times, such as from about 10 to 15 minutes, may be used depending on existing damage to the hair, such as if it was previously bleached. In some cases, the peptides or hair composition thereof (step 2) are applied to the hair for a processing period of about 5 minutes. Any step can also optionally be repeated more than once. Hair can optionally be washed and dried before or after any step.

The hair keratin relaxation composition (of step 1) or the peptides or hair composition thereof (of step 2) can be applied using a brush and/or comb. Optionally, all the hair fibers being straightened or shaped are wetted by the compositions used to ensure the hair fibers are saturated. Optionally, following steps 1, 2, and 3, a leave-in conditioner may be applied to the hair for a processing period of at least about 5 minutes followed by optionally drying the hair.

A variety of different methods may be used for relaxation (step 1) and fixation (step 3). In step 1, any of the following may be used to break disulfide bonds: (1) heat; (2) applying a lye relaxer (e.g., sodium hydroxide solution at pH 12-14); (3) applying a no-lye relaxer (e.g., calcium hydroxide or guanidine carbonate at pH 9-11); (4) applying a “thio” relaxer (e.g., sodium thioglycolate, ammonium thioglycolate, dithiothreitol, thioglycolic acid, thiolactic acid, dihydrolipoate, thioglycerol, mercaptopropionic acid, pegylated thiols); (5) applying a natural, thiol-bearing compound (e.g., glutathione, cysteine, cysteinyl-glycine) optionally along with ascorbic acid or its derivates, preferably in a mildly basic solution (e.g., pH 9-10); or (6) applying an enzyme known to play a role in reducing keratin disulfide bonds with its necessary substrate(s) (e.g., glutathione reductase, cysteine dioxygenase, alkyl hydroperoxide reductase, thioredoxin reductase, dihydrolipoyl dehydrogenase, peptide methionine sulfoxide reductase, phospho-adenosine phosphosulfate reductase, ribonucleoside-diphosphate reductase). Non-thio relaxers are also known and may used in lieu of “thio” relaxers including, but not limited to, alkaline hydroxides (i.e., sodium hydroxide, potassium hydroxide), sodium bisulfite, ammonium bisulfide, zinc formaldehyde sulfoxylate, sodium formaldehyde sulfoxylate, sodium metabisulfite, potassium borohydride, and hydroquinone.

For fixation, as takes place in step 3, any of the following may be used to form or reform disulfide bonds: (1) applying sufficient heat; (2) application of an acid or acidic solution; (3) application of hydrogen peroxide; (4) application of any oxidase enzyme that produces hydrogen peroxide with its necessary substrate(s) (e.g., glucose oxidase, etc.); and/or (5) not washing the hair for at least 24 hours after step 2, optionally also adding a conditioning agent to the hair. In some instances, applying sufficient heat to the hair during fixation can involve blow drying and/or flat ironing the hair at a temperature ranging from about 375 to about 410° F. or about 350 to about 375° F. using a suitable number of passes, such as about 5 to 8 passes. In some cases, lower heating temperatures may be used depending on existing damage to the hair, such as if it was previously bleached.

The bond shaping formulation containing the peptide may be applied to hair following a hair treatment (i.e., a post-treatment application), such as a hair coloring, hair bleaching, permanent hair waving, hair straightening, or other common hair treatment. In some other instances, the bond shaping formulation may be applied to the hair, prior to a hair treatment (i.e., a pre-treatment application), such as a hair coloring, hair bleaching, permanent hair waving, hair straightening, or other common hair treatment. Hair coloring is art understood to refer to treatments that modify the color of hair, such as using oxidative dyes and precursors thereof, or direct dyes. Hair bleaching is art understood to refer to treatments that remove color from hair such as by application of bleaching agents. Bleaching agents used in bleaching methods include applying a combination of developer (aqueous hydrogen peroxide) and bleach powder (a powder containing at least persulfate(s) and alkalizing agent(s)). The aforementioned treatments are well known to the person of skilled in the art. Following a treatment, the bond shaping formulation, in any suitable form, can be applied on the same day or it may be applied later, such as within 1 to 2 weeks following treatment. The amount of the bond shaping formulation applied can, in some instances, be sufficient to saturate the hair. The bond shaping formulation may be applied to the hair as a single application, or application may be repeated one or more times. The volume of bond shaping formulation applied to the hair in each application may be about 1 to about 100 mL per person depending on their length and volume of hair. In some embodiments, application of the bond shaping formulation could be repeated immediately (e.g., within 10 to 15 seconds) or approximately 1, 5, 7.5, 10, 12.5, 15, 17.5, or 20 minutes after the first application. In some instances, it may be necessary to remove the bond shaping formulation from the hair following an application, such as by rinsing and/or shampooing the hair following application, for example within 10, 15, 25, 30, 45, or 60 seconds, or within two, three, four, or five minutes after application. Alternatively, the bond shaping formulation may be rinsed from the hair within about 30 minutes following application, between about 5 minutes and about 20 minutes, or about 10 minutes after application of the bond shaping formulation to the hair, depending on hair type.

The above description is presented to enable a person skilled in the art to make and use the disclosed peptides and is provided in the context of a particular application and its requirements. Various modifications to the embodiments and aspects will be readily apparent to those skilled in the art, and the generic principles defined herein may be applied to other aspect, embodiments and applications without departing from the spirit and scope of the information disclosed herein. The examples herein are not intended to be limited to the aspects or embodiments described but are to be accorded the widest scope consistent with the principles and features disclosed herein. A further understanding can be obtained by reference to certain specific examples.

The following peptides were synthesized and provided by a commercial supplier (Genscript USA, Inc., Piscataway, NJ): SEQ ID NO: 3-SEQ ID NO: 108.

The disclosed compositions and methods of making and using thereof can be further understood through the following enumerated paragraphs.

Paragraph 1. A kit for defining curls comprising:
(a) a bond shaping formulation comprising a peptide; and
(b) a bond building formulation comprising bis-aminopropyl diglycol dimaleate.

Paragraph 2. The kit of paragraph 1, wherein the peptide comprises an amino acid sequence of any one of SEQ ID NOS: 3.-SEQ ID NOS: 17., SEQ ID NOS: 18-SEQ ID NOS: 109, or a fragment or variant of any one of SEQ ID NO:1 and SEQ ID NOS: 3.-SEQ ID NOS: 17., SEQ ID NOS: 18-SEQ ID NOS: 109.

Paragraph 3. The kit of paragraph 1 or 2, wherein the peptide is SEQ ID NO: 8 or a variant thereof with at least 70% sequence identity thereto.

Paragraph 4. The kit of any one of paragraphs 1-3, wherein the peptide consists of SEQ ID NO: 8.

Paragraph 5. The kit of any one of paragraphs 1-4, wherein the bond shaping formulation and bond building formulation each further comprises one or more cosmetically acceptable carriers and one or more cosmetically acceptable excipients.

Paragraph 6. The kit of paragraph 5, wherein the peptide is present in an amount from about 0.01 to about 0.1% wt of the bond shaping formulation, and the binding agent is present in an amount from about 0.01 wt % to about 1 wt %, from about 0.01 wt % to about 0.5 wt %, from about 0.01 to about 0.1 wt %, or from about 0.01 to about 0.05 wt % of the bond building formulation.

Paragraph 7. The kit of any one of paragraphs 1-6, further comprising an enhancing formulation comprising water, an alcohol (optionally propanediol), a chelating agent, a surfactant, an emollient, an emulsifier, and optionally a fragrance.

Paragraph 8. The kit of paragraph 7, wherein the total amount of the chelating agent, surfactant, emollient, and emulsifier is less than 0.1 wt % of the enhancing formulation.

Paragraph 9. The kit of paragraph 7 or 8, wherein the enhancing formulation comprises water, propanediol, caprylyl/capryl glucoside, sodium cocoyl glutamate, citric acid, trisodium ethylenediamine disuccinate, polyglyceryl-6 oleate, glyceryl caprylate, and sodium surfactin.

Paragraph 10. A method for defining curls, comprising:
(1) applying a bond shaping formulation to hair, wherein the bond shaping formulation comprises a peptide; and
(2) applying a bond building formulation to the hair, wherein the bond building formulation comprises bis-aminopropyl diglycol dimaleate.

Paragraph 11. The method of paragraph 10, wherein the peptide comprises an amino acid sequence of any one of SEQ ID NOS: 3.-SEQ ID NOS: 17., SEQ ID NOS: 18-SEQ ID NOS: 109, or a fragment or variant of any one of SEQ ID NO:1 and SEQ ID NOS: 3.-SEQ ID NOS: 17., SEQ ID NOS: 18-SEQ ID NOS: 109.

Paragraph 12. The method of paragraph 10 or 11, wherein the peptide is SEQ ID NO: 8 or a variant thereof with at least 70% sequence identity thereto.

Paragraph 13. The method of any one of paragraphs 10-12, wherein the peptide consists of SEQ ID NO: 8.

Paragraph 14. The method of any one of paragraphs 10-13, wherein in step (1), the bond shaping formulation is applied to the hair by an applicator bottle.

Paragraph 15. The method of any one of paragraphs 10-14, further comprising, in step (1) or subsequent to step (1), step (i) raking through the hair in a downward motion to ensure the hair is fully saturated with the bond shaping formulation.

Paragraph 16. The method of any one of paragraphs 10-15, wherein the bond shaping formulation is left on the hair for a time period of about 1 min to about 20 mins or from about 5 mins to about 15 mins, such as about 10 mins, following step (1).

Paragraph 17. The method of any one of paragraphs 10-16, further comprising, subsequent to step (1) and prior to step (2), step (1a) applying an enhancing formulation to the hair, wherein the enhancing formulation comprises water, an alcohol (optionally propanediol), a chelating agent, a surfactant, an emollient, an emulsifier, and optionally a fragrance.

Paragraph 18. The method of paragraph 17, wherein in step (1a), the enhancing formulation is sprayed to saturate the hair with the enhancing formulation.

Paragraph 19. The method of paragraph 17 or 18, further comprising shaping the hair, such as by squeezing, compressing, or scrunching, to define the curls during step (1a) and/or subsequent to step (1a).

Paragraph 20. The method of any one of paragraphs 17-19, wherein the enhancing formulation is left on the hair for a time period of about 1 minute to about 20 minutes or from about 5 minutes to about 15 minutes, such as about 10 minutes, following step (1a).

Paragraph 21. The method of any one of paragraphs 17-20, wherein step (1a) is performed one or more times during step (2).

Paragraph 22. The method of any one of paragraphs 10-21, further comprising, prior to step (1), step (3) rinsing the hair with water or washing the hair with a shampoo.

Paragraph 23. The method of any one of paragraphs 10-22, further comprising, subsequent to step (2), step (4) shaping the curls into a desired shape, optionally with a curl defining tool, such as curlers, rollers, curling rod, and/or a curl defining brush.

Paragraph 24. The method of any one of paragraphs 10-23, further comprising, subsequent to step (2), step (5) drying the hair.

Paragraph 25. The method of any one of paragraphs 10-24, wherein subsequent step (2), the hair is not rinsed or washed for at least 24 hours, at least 36 hours, at least 48 hours, at least 60 hours, or at least 72 hours.

Paragraph 26. The method of any one of paragraphs 10-25, wherein following step (2), and following drying of the hair, the curls are shinier, bouncier, and/or more defined as compared to untreated hair from the same individual, by visual inspection.

Paragraph 27. The method of any one of paragraphs 10-26, wherein following step (2), the hair is stronger compared to untreated hair from the same individual or hair from the same individual that is washed with a non-conditioning shampoo, such as 15% SLES solution.

Paragraph 28. The method of any one of paragraphs 10-27, wherein following step (2), the hair has reduced breakage compared to untreated hair from the same individual or hair from the same individual that is washed with a non-conditioning shampoo, such as 15% SLES solution.

Paragraph 29. The method of any one of paragraphs 10-28, wherein following step (2), and following drying of the hair, the hair has reduced frizz compared to untreated hair from the same individual or hair from the same individual that is washed with a non-conditioning shampoo, such as 15% SLES solution.

### Example 1: Treatment with Heat Only

Tresses of curly Brazilian hair were cut into 1/2 inch wide samples. The tresses were then washed with shampoo and allowed to air dry. The hair was straightened using a flat iron at 460° F. Once the hair was visibly straight, the hair was wrapped tightly in aluminum foil. A flat iron was applied to a section of the wrapped hair by holding the flat iron to the section for 10 seconds. This process was repeated until all sections of the hair were flat ironed.

Six individual solutions of each peptide corresponding to SEQ ID NO. 3 through SEQ ID NO 8 were dissolved in de-ionized (“DI”) water at a concentration of about 1 milligram per milliliter (“mg/mL”) and then applied to separate hair samples until the hair sample was saturated. The saturated hair was allowed to sit for 1 hour at 37° C. The hair was then blow dried under medium heat while gently pulling the hair into a straight shape. A flat iron was then applied to the hair at 400° F.

The straightened hair was washed with shampoo and allowed to air dry, and this washing/drying procedure was repeated a total of 10 times. Following this treatment, each hair sample was significantly straightened compared to untreated samples, and small waves remained in the treated hair. There was little identifiable difference in efficacy among the six designed keratin binding peptides, although peptides corresponding to SEQ ID NO. 5 SEQ ID NO. 6, SEQ ID NO. 7 and SEQ ID NO. 8 showed a slightly higher straightening efficiency than SEQ ID NO. 3 and SEQ ID NO.4.

### Example 2: Treatment with Lye and Hydrogen Peroxide

Tresses of curly Brazilian hair were cut into ½ inch wide samples. The tresses were then washed with shampoo and allowed to air dry. The hair then soaked in a solution of sodium hydroxide pH 12 for 30 minutes at room temperature. After this time, the hair was rinsed with DI water. The hair was then blow dried under medium heat while gently pulling the hair into a straight shape. A flat iron was then applied to the hair at 400° F.

Six individual solutions of each designed keratin binding peptide corresponding to SEQ ID NO. 3 through SEQ ID NO. 8 were dissolved in in DI water at a concentration of 1 mg/ml and then applied to individual hair samples until the hair sample was saturated, and the hair sample was allowed to sit for 30 minutes at room temperature. The hair sample was then blow dried under medium heat while gently pulling the hair into a straight shape. A flat iron was then applied to the hair at 400° F.

L'Oreal® Oreor Creme 40 volume developer was then applied to the hair samples until the hair sample was saturated, and then allowed to sit for 30 minutes at room temperature. The hair samples were then rinsed with DI water.

Each straightened hair sample was washed with shampoo and allowed to air dry, and this washing/drying procedure was repeated a total of 10 times. Following this treatment, each hair sample was significantly straightened compared to untreated samples, and only slight waves remained in the treated hair. There was little identifiable difference in efficacy among the six peptides, although peptides corresponding to SEQ ID NO. 5, SEQ ID NO. 6, SEQ ID NO. 7 and SEQ ID NO. 8 showed a slightly higher straightening efficiency than SEQ ID NO. 3 and SEQ ID NO.4.

### Example 3: Treatment with Lye and Glucose Oxidase

Tresses of curly Brazilian hair were cut into ½ inch wide samples. The tresses were then washed with shampoo and allowed to air dry. The hair then soaked in a solution of sodium hydroxide pH 12 for 30 minutes at room temperature. After this time, the hair was rinsed with DI water. The hair was then blow dried under medium heat while gently pulling the hair into a straight shape. A flat iron was then applied to the hair at 400° F.

A solution of the peptide corresponding to SEQ ID. NO. 8 in DI water at a concentration of 1 mg/mL was then applied to hair sample until the hair sample was saturated, and the hair was allowed to sit for 30 minutes at room temperature. The hair was then blow dried under medium heat while gently pulling the hair into a straight shape. A flat iron was then applied to the hair at 400° F.

A solution of glucose oxidase (2 mg/mL) and glucose (18 mg/mL) in 50 mM sodium phosphate buffer pH 5.8 was then applied to the hair until the hair was saturated, and the hair was allowed to sit for 30 minutes at room temperature. The hair was then rinsed with DI water.

The straightened hair was washed with shampoo and allowed to air dry, and this washing/drying procedure was repeated a total of 10 times. Following this treatment, each hair sample was significantly straightened compared to untreated samples, and only slight waves remained in the treated hair.

### Example 4: Treatment with Glutathione Reductase and Hydrogen Peroxide

Tresses of curly Brazilian hair were cut into ½ inch wide samples. The tresses were then washed with shampoo and allowed to air dry. The hair then soaked in a solution of glutathione reductase (0.01 mg/mL), glutathione (100 mg/mL), and nicotinamide adenine dinucleotide phosphate (0.083 mg/mL) in 50 mM sodium phosphate buffer pH 8 for 30 minutes at room temperature. After this time, the hair was rinsed with DI water. The hair was then blow dried under medium heat while gently pulling the hair into a straight shape. A flat iron was then applied to the hair at 400° F.

A solution of a peptide corresponding to SEQ ID NO.8 in DI water at a concentration of 1 mg/mL was then applied to har until the hair was saturated, and the hair was allowed to sit for 30 minutes at room temperature. The hair was then blow dried under medium heat while gently pulling the hair into a straight shape. A flat iron was then applied to the hair at 400° F.

L'Oreal® Oreor Creme 40 volume developer was then applied to the hair until the hair was saturated, and the hair was allowed to sit for 30 minutes at room temperature. The hair was then rinsed with DI water.

The straightened hair was washed with shampoo and allowed to air dry, and this washing/drying procedure was repeated a total of 10 times. Following this treatment, each hair sample was significantly straightened compared to untreated samples, and only slight waves remained in the treated hair.

### Example 5: Treatment with Glutathione and Glucose Oxidase

Tresses of curly Brazilian hair were cut into ½ inch wide samples. The tresses were then washed with shampoo and allowed to air dry. The hair then soaked in a solution of glutathione (100 mg/mL) in sodium hydroxide pH 9 for 30 minutes at room temperature. After this time, the hair was rinsed with DI water. The hair was then blow dried under medium heat while gently pulling the hair into a straight shape. A flat iron was then applied to the hair at 400° F.

A solution of the peptide corresponding to SEQ ID NO.8 in DI water at a concentration of 1 mg/mL was then applied to hair until the hair was saturated, and the hair was allowed to sit for 30 minutes at room temperature. The hair was then blow dried under medium heat while gently pulling the hair into a straight shape. A flat iron was then applied to the hair at 400° F.

A solution of glucose oxidase (2 mg/mL) and glucose (18 mg/mL) in 50 mM sodium phosphate buffer pH 5.8 was then applied to the hair until the hair was saturated, and the hair was allowed to sit for 30 minutes at room temperature. The hair was then rinsed with DI water.

The straightened hair was washed with shampoo and allowed to air dry, and this washing/drying procedure was repeated a total of 10 times. Following this treatment, each hair sample was significantly straightened compared to untreated samples, and only slight waves remained in the treated hair.

### Example 6: Treatment with Ammonium Thioglycolate and Hydrogen Peroxide

Tresses of curly Brazilian hair were cut into ½ inch wide samples. The tresses were then washed with shampoo and allowed to air dry. The hair then soaked in a solution of ammonium thioglycolate (10% v/v) in sodium hydroxide pH 10 for 30 minutes at room temperature. After this time, the hair was rinsed with DI water. The hair was then blow dried under medium heat while gently pulling the hair into a straight shape. A flat iron was then applied to the hair at 400° F.

In some samples, a solution of the peptide corresponding to SEQ ID NO.8 in DI water at a concentration of 1 mg/mL was then applied to hair until the hair was saturated, and the hair was allowed to sit for 30 minutes at room temperature. In these samples, the hair was then blow dried under medium heat while gently pulling the hair into a straight shape. A flat iron was then applied to the hair at 400° F.

For all hair samples, a solution of hydrogen peroxide (2% v/v) was then applied to the hair until the hair was saturated, and the hair was allowed to sit for 30 minutes at room temperature. The hair was then rinsed with DI water.

The straightened hair was washed with shampoo and allowed to air dry, and this washing/drying procedure was repeated a total of 30 times. Following this treatment, the treated hair samples were significantly straightened compared to an untreated sample, and only slight waves remained in the treated hair.

The break force of treated hair samples was measured using a custom apparatus. Individual hairs were cut into 13-centimeter-long sections. Both ends of the hairs were wrapped in tape 1 centimeter from each end. The hair was then held in a vertical position between two clamps (top=fixed, bottom=movable) with 1-centimeter-long grips such that the grips were aligned with the taped ends of the hair. Force was gradually applied to the bottom clamp, and the force at break was recorded. Ten replicates were collected for each treatment group.

### Example 7: Fluorescence Binding Studies

To a solution of 500 μL of DI water was added 1 mg of SEQ ID NO. 8. Then, a solution of 84 mg of sodium bicarbonate in 1 mL of DI water was prepared, and 12 μL of this solution was added to the SEQ ID NO. 8 solution. Immediately prior to conjugation, 1 mg of Alexa Fluor 647 NHS ester (Thermo Fisher Scientific) dye molecule was dissolved in 100 μL of DMSO, and 15 μL of this solution was added to the SEQ ID NO. 8 solution. After the dye was added, the solution was incubated at 4° C. in the dark for 16 hours. Removal of unbound dye molecules was carried out using a PD MiniTrap G-10 column (Cytiva) using DI water as the elutant. Fractions containing dye labeled SEQ ID NO. 8 were pooled.

Hair samples were then treated according to Example 6, except that treatment with SEQ ID NO. 8 was replaced with dye labeled SEQ ID NO. 8 while varying the peptide concentration), binding time, number of SEQ ID NO. 7 applications, or number of shampoos at the end of the treatment.

Fluorescence images were collected on a EVOS M7000 Imaging System (Thermo Fisher Scientific) using a Cy5 filter set. Images were collected using a 0.1 second exposure time at a light intensity of 0.0023.

### Example 8: Comparative Tensile Studies

Swatches of Brazilian hair were bleached using BW2 hair powder lightener (bleach powder) (Clariol) and oreor creme 40 volume developer (aqueous hydrogen peroxide) (L'Oreal) mixed in a 1:2 ratio with constant stirring for 1 minute until the bleaching mixture became smooth and homogeneous. The hair swatches were saturated in the bleaching mixture and left to sit at room temperature for 45 minutes. The hair swatches were then rinsed with deionized water (DI) for 2 minutes, thoroughly shampooed, and allowed to air dry. The bleaching procedure was repeated two additional times to yield bleached Brazilian hair swatches.

Tresses of both virgin and bleached curly Brazilian hair were cut into ½ inch wide samples. The tresses were then washed with shampoo and allowed to air dry. The hair then soaked in a solution of cysteine (100 mg/mL) and ascorbic acid (10 mg/mL) in sodium hydroxide pH 10 for 30 minutes at room temperature. After this time, the hair was rinsed with DI water. The hair was then blow dried under medium heat while gently pulling the hair into a straight shape. A flat iron was then applied to the hair at 400° F.

In some samples, a solution of the peptide corresponding to SEQ ID NO. 1, SEQ ID NO. 8, SEQ ID NO. 9, or SEQ ID NO. 10 in DI water at a concentration of 1 mg/mL was then applied to hair until the hair was saturated, and the hair was allowed to sit for 30 minutes at room temperature. The hair was then blow dried under medium heat while gently pulling the hair into a straight shape. A flat iron was then applied to the hair at 400° F.

For all hair samples, a solution of hydrogen peroxide (2% v/v) was then applied to the hair until the hair was saturated, and the hair was allowed to sit for 30 minutes at room temperature. The hair was then rinsed with DI water.

The straightened hair was washed with shampoo and allowed to air dry. The break force of treated hair samples was measured using a custom apparatus. Individual hairs were cut into 13-centimeter-long sections. Both ends of the hairs were wrapped in tape 1 centimeter from each end. The hair was then held in a vertical position between two clamps (top=fixed, bottom=movable) with 1-centimeter-long grips such that the grips were aligned with the taped ends of the hair. Force was gradually applied to the bottom clamp, and the force at break was recorded. Ten replicates were collected for each treatment group.

Each hair sample was then washed with shampoo an additional 9 times and allowed to air dry. The break force for each hair sample was then remeasured following these additional washes using ten replicates for each sample.

### Example 9: Measurement of Peptide Stability to Shampoo Washes

To separate solutions of 500 μL of DI water was added 1 mg of one of the peptides corresponding to SEQ ID NOs. 1 and 2-17. Then, a solution of 84 mg of sodium bicarbonate in 1 mL of DI water was prepared, and 12 μL of this solution was added to each peptide solution. Immediately prior to conjugation, 5 mg of Alexa Fluor 647 NHS ester (Thermo Fisher Scientific) dye molecule was dissolved in 500 μL of DMSO, and 15 μL of this solution was added to each peptide solution. After the dye was added, the solution was incubated at 4° C. in the dark for 16 hours. Removal of unbound dye molecules was carried out using a PD MiniTrap G-10 column (Cytiva) using DI water as the elutant. Fractions containing dye-labeled peptide were pooled and diluted to final peptide concentrations of 1 mg/mL.

Hair samples were then treated according to Example 8, except that during treatment with peptide, hair was treated with a single dye-labeled peptide corresponding to SEQ ID NOs. 1 and 2-17. For each set of conditions, three separate straightening protocols were performed.

Fluorescence images were collected on a EVOS M7000 Imaging System (Thermo Fisher Scientific) using a Cy5 filter set. Images were collected using a 0.1 second exposure time at a light intensity of 0.0023. Following fluorescence measurements, hair samples were then washed with shampoo an additional 9 times, towel dried, and fluorescently imaged again.

Image files were processed as 16-bit grayscale images using ImageJ software. A lower threshold of 497 intensity units was first applied to each image to remove background intensity from areas that did not contain hair strands. The average and standard deviation of fluorescence intensity in the remaining regions (corresponding to hair strands containing fluorescently labeled peptides) was then directly calculated.

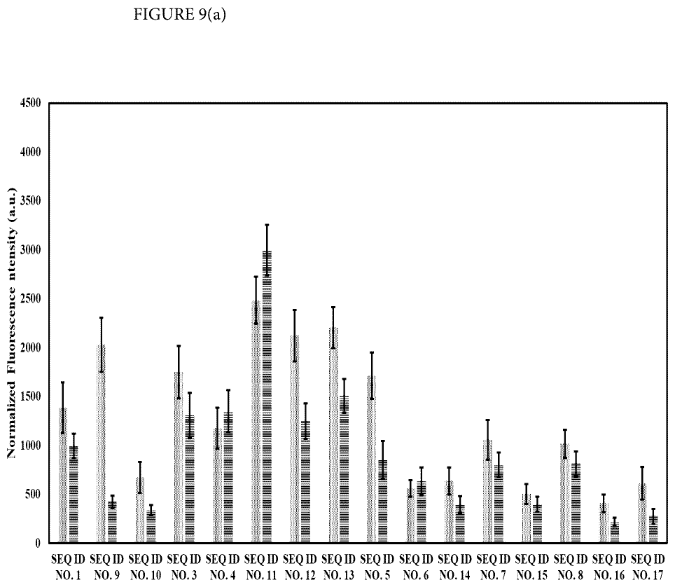

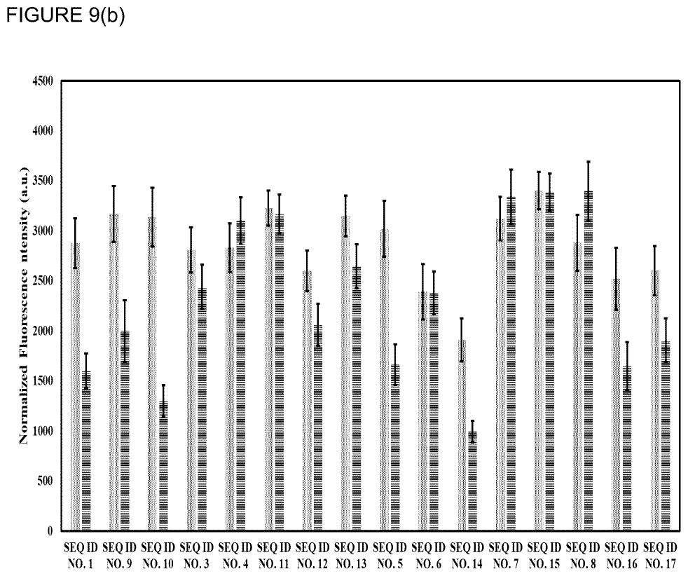

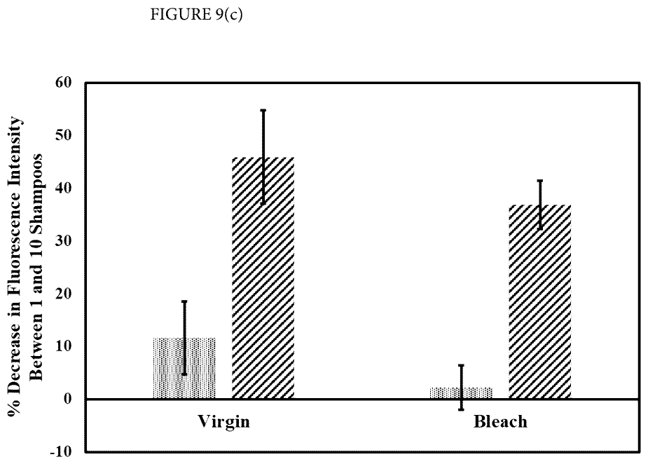

Analysis of the peptides that showed strong resistance to shampoo washing, as indicated by no statistically significant decrease in fluorescence intensity between 1 and 10 shampoos in virgin (FIG. 9a) and bleached (FIG. 9b) hair, revealed that almost all of these peptides had a Q13C substitution relative to SEQ ID NO. 1. Indeed, peptides with this substitution showed a significantly smaller percentage loss from hair after 10 shampoo washes (FIG. 9c). Furthermore, SEQ ID NOs. 4, 11, 12, and 13 all have the Q13C substitution relative to SEQ ID NO. 1 and differ in sequence only by the number of other cysteine substitutions at common sites (3, 2, 1, and 0 additional cysteine substitutions, respectively). However, only hair treated with SEQ ID NOs. 4 and 11 shows no statistically significant decrease in fluorescence intensity between 1 and 10 shampoos, suggesting that a minimum of 2 non-Q13C cysteine substitutions may be required to allow the peptides to prevent loss from hair upon repeated shampooing. SEQ ID NOs. 14, 15, and 16 differ from SEQ ID NOs. 6, 7, and 8, respectively, in that the former sequences lack the Q13C substitutions found in the latter respective sequences. While hair treated with all of the latter peptides showed no statistically significant decrease in fluorescence intensity between 1 and 10 shampoos, SEQ ID NOs. 14 and 16 showed a significant decrease, generally supporting the importance of the Q13C substitution relative to SEQ ID NO. 1 in promoting strong binding to hair such that the peptide is not removed from hair during shampoo washes. The strong retention of SEQ ID NO. 15 in hair through 10 shampoos despite lacking the Q13C substitution could suggest that the large number of cysteine substitutions in this peptide relative to SEQ ID NO.1 (10 cysteine substitutions) or the unique cysteine substitutions in this peptide, Q3C and/or Q8C, also allow strong binding to hair.

### Example 10: Measurement of Retention in Hair for Library of Peptides following Shampoo Washes

A 1 g sample of Brazilian hair was washed with 70% ethanol and incubated at room temperature in 20 mL of a 2:1 chloroform: methanol (v/v) solution for 16 hours to remove lipids from the hair. The hair was then thoroughly rinsed with DI water and added to a 20 mL solution of 100 mM Tris buffer pH 8.0 containing 8 M urea and 10% 2-mercaptoethanol (v/v). The hair was incubated at 50° C. for 5 days in this mixture, after which time the mixture was filtered. The filtrate was dialyzed against DI water at 4° C. using 8 exchanges with at least 3 hours between each exchange. The mixture was centrifuged at 10,000×g for 30 minutes at 4° C., the supernatant was collected, and the concentration of keratin proteins in the supernatant was measured using a BCA protein assay (Thermo Fisher Scientific).

The keratin mixture was diluted to a concentration of 5 mg/mL, and to 1 mL of this solution was added 60 μL of a solution of 84 mg of sodium bicarbonate in 1 mL of DI water. Immediately prior to conjugation, 1 mg of Alexa Fluor 647 NHS ester (Thermo Fisher Scientific) dye molecule was dissolved in 100 μL of DMSO, and 81 μL of this solution was added to the keratin solution. After the dye was added, the solution was incubated at 4° C. in the dark for 16 hours. Removal of unbound dye molecules was carried out using a PD MiniTrap G-10 column (Cytiva) using DI water as the elutant. Fractions containing dye-labeled keratin proteins were pooled.

A 4 mg sample of each of the 92 peptides corresponding to SEQ ID NOs.: 18-109, which contained between 1 and 12 cysteine substitutions and between 0 and 4 alanine substitutions relative to SEQ ID NO. 1, was dissolved in 1 mL of DI water. A 50 μL sample of each solution was then added to a single well in 3 separate Nunc Covalink NH 96 well plates (Thermo Fisher Scientific), in which the wells are functionalized with free amines, such that three replicates could be obtained in future experiments. Then, 5.52 mg of N-hydroxysulfosuccinimide was dissolved in 15 mL of DI water, and 50 μL of this solution was added to each well. Then, 18.45 mg of N-(3-Dimethylaminopropyl)-N′-ethylcarbodiimide hydrochloride was dissolved in 15 mL of DI water, and 50 μL of this solution was added to each well. The well plates were incubated at room temperature for 2 hours. Each well was then washed 5 times with DI water, using a 15-minute incubation time for the final wash.

Keratin proteins labeled with Alexa Fluor 647 was diluted to a concentration of 0.2 mg/mL in sodium hydroxide pH 10, and 80 μL of this solution was added to each well. The well plates were incubated for 1 hour at 37° C. Each well was then washed 5 times with DI water, using a 15-minute incubation time for the final wash. The wells were then incubated at room temperature with a 10% solution of shampoo for 5 minutes, washed 3 times with DI water, and allowed to air dry in the dark.

Fluorescence images were collected for each well in the three replicate well plates on a EVOS M7000 Imaging System (Thermo Fisher Scientific) using a Cy5 filter set. Images were collected using a 0.1 second exposure time at a light intensity of 0.15. Following fluorescence measurements, wells were subject to 9 additional cycles of 5 minutes incubations at room temperature in 10% shampoo. The wells were then washed 3 times with DI water, air dried in the dark, and fluorescently imaged again.

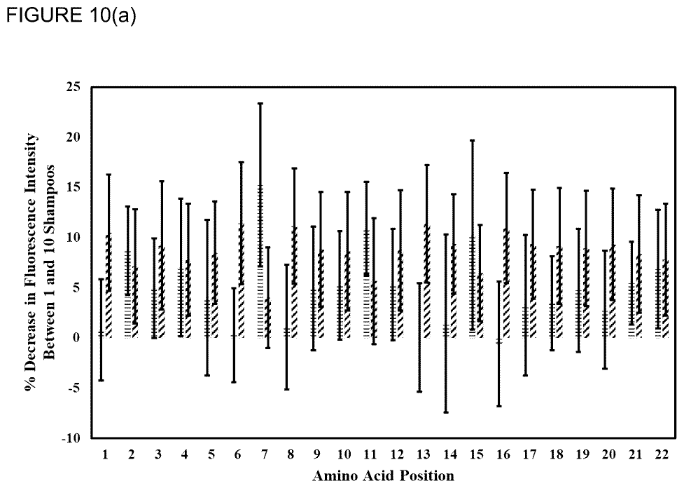

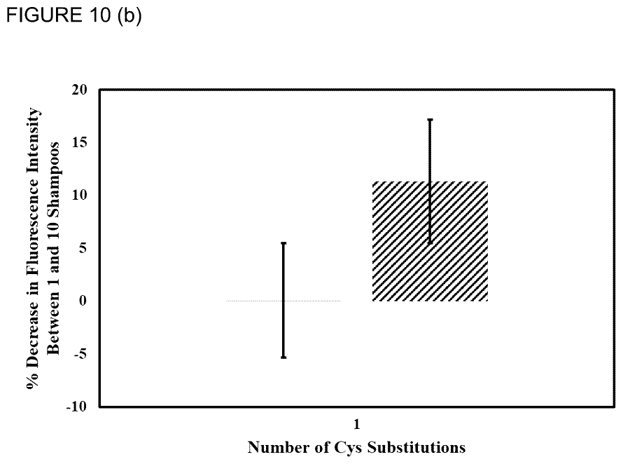

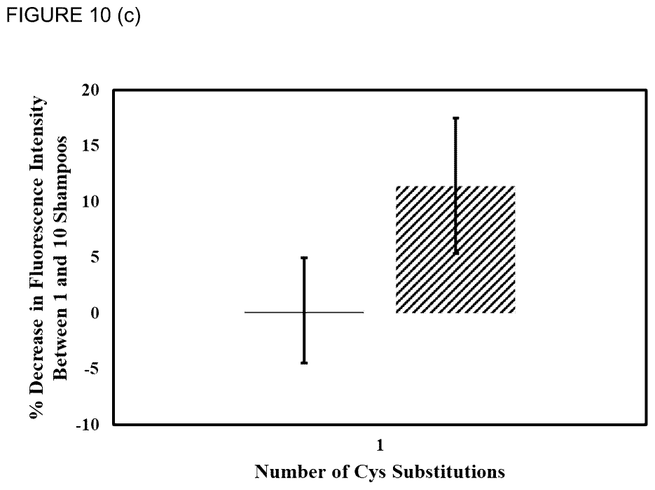

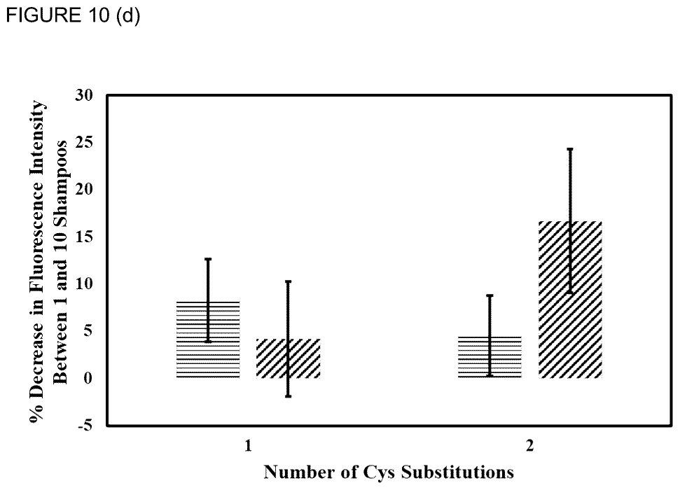

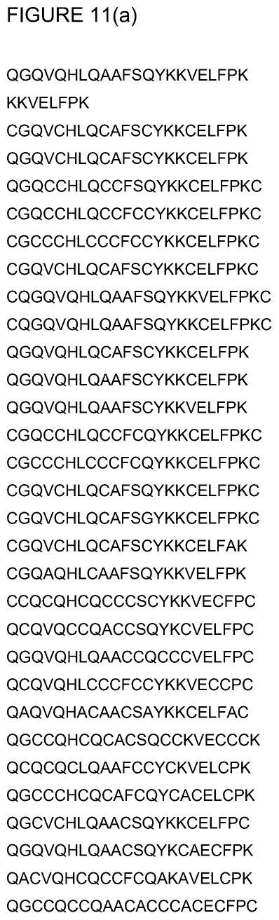

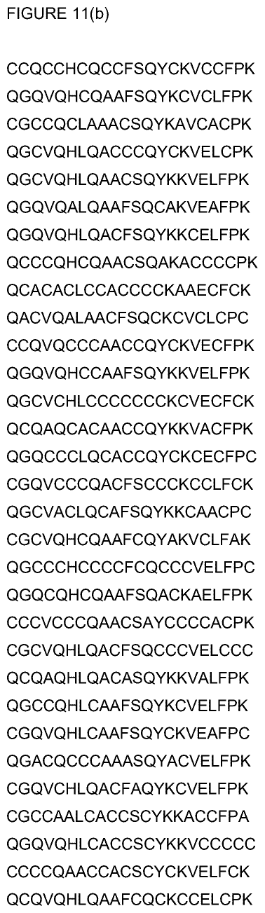

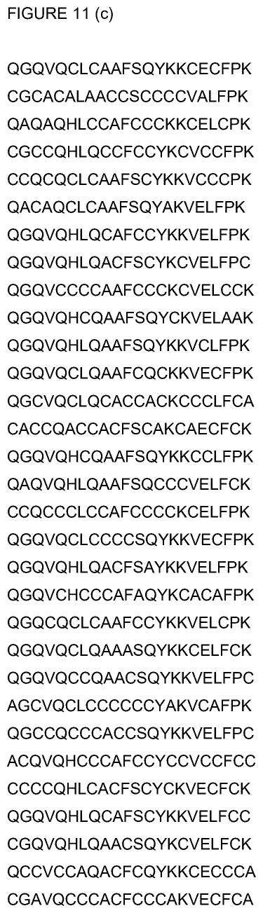

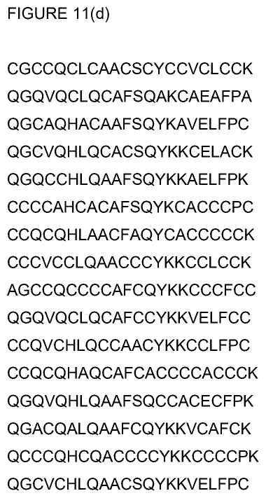

Analysis of the results demonstrated that peptides with either the Q13C or H6C substitutions showed significantly stronger binding against repeated shampoo washes than peptides without these substitutions (FIG. 10a). When the Q13C substitution was present in a peptide, at least 1 additional cysteine substitution was needed to observe a statistically significant improvement in resistance to shampoo washes (FIG. 10b). Similarly, when the H6C substitution was present in a peptide, at least 1 additional cysteine substitution was needed to observe a statistically significant improvement in resistance to shampoo washes (FIG. 10c). When neither the Q13C nor the H6C substitutions were present, peptides with 3 or more cysteine substitutions demonstrated significantly greater resistance to repeated shampoo washes than peptides with fewer than 3 cysteine substitutions or the Q13C and/or the H6C substitution (FIG. 10d).

### Example 11: Curl Defining and/or Rebuilding Treatment

### Exemplary Formulations

Bond shaping formulation: Purified Water, Natrosol 250 HHR CS, Glycerin 99.7%, Natrlquest E30, Mikrokill COS, PB-300, S G M FRESHENYOU 137V19, Citric Acid, Caustic Soda Micropearls.

Enhancing formulation: Purified Water, Zemea Propanediol, Natrlquest E30, Citric Acid, Hydrogen Peroxide-35% Solution, SymbioSolv Clear Plus MB, Sugared Grapefruit & Mint 138V14.

Bond building formulation: Purified Water, Aristoflex Silk, Gantrez S-97 BF, Glycerin 99.7%, Sodium Hydroxide 20% Solution, Natrosol 250 HHR CS, Citric Acid 50% Solution, Natrlquest E30, Aquaflex XL-30, Xylishine, PlantCol COS, Lincoserve PE-815, Coconut-Avocado Hair Milk, TeraSolve, Sugared Grapefruit & Mint 138V14, Gelest XG-2820.

### Treatment Steps for Stylist and Client Perception Tests

PREPARATION: Before treatment, hair was cleaned hair with shampoo (such as No. 4C BOND MAINTENANCE® CLARIFYING SHAMPOO) for up to 5 minutes to remove product buildup and damaging impurities. Optionally, following cleaning with the shampoo, the hair is further cleaned with a deep cleaning product, such as BROAD SPECTRUM CHELATING TREATMENT.

### STEP 1: REPAIR

50 mL* bond shaping formulation was dispensed into an applicator bottle. Hair was sectioned into 4 quadrants and the bond shaping formulation was applied in small sections until hair was evenly saturated. The hair was raked through in a downward motion to ensure product was fully saturated. Processed for 10 minutes. No rinse.

*Amount depends on hair length and density. For short lengths, use less product. For long or dense lengths, add more product as needed.

### STEP 2: ENHANCE

Small sections of hair was sprayed with the enhancing formulation until hair was thoroughly saturated. Hair was scrunched into curls to ensure product was properly worked through. Processed for 10 minutes. No rinse.

### STEP 3: DEFINE

Starting with a small amount, the bond building formulation was evenly distributed, section by section, from root to tip. The enhancing formulation was misted to re-wet hair if needed. Hair was treated with desired curl-defining technique, and dried (air dry or other preferred drying method). Hair was left without wash for 48 hours.

TABLE 1
Summary of Results from Stylist Perception Tests
STYLIST PERCEPTION (Percentage of Stylists Agree)
COMBINED RESULTS

100% of professionals agree products are easy
to apply, distribute and work through curls,
treatment is easy to perform and follow.
99% of professionals agree client's curls
are revived, defined, moisturized & shiny
97% of professionals agree client's curls
look repaired & visibly improved
94% of professionals agree treatment helped improve
curl retention & restore elasticity and bounce

TABLE 2
Summary of Results from Client Perception Tests
CLIENT PERCEPTION (Percentage of Clients Agree)
COMBINED RESULTS- IMMEDIATELY AFTER TREATMENT

97% of clients agree curls look restored and revived
96% of clients agree curls look and feel healthier,
repaired, hydrated & frizz-free

Clinical tests on hair swatches were also conducted. Sections I-V below describe details of each test listed in Table 3, including experimental procedures and overall results.

TABLE 3

| | Tests Performed |
|---|---|
| Summary of Data | (see Sections I-V) |
| Clinically proven to | Curl Droop Test |
| strengthen hair and improve | (see Section I) |
| curl retention after 1 treatment | and Anti-Breakage Test |
| | (see Section II) |
| Clinically proven to improve | Curl Droop Test |
| curl retention after 1 treatment | (see Section I) |
| Reduces frizz after 1 treatment | Anti-Frizz Test |
| Humidity resistant | (see Section III) |
| Controls frizz through 15 washes | |
| Significant improvement in conditioning, | Wet Combing Test |
| detangling, smoothness | (see Section IV) |
| Strengthens hair/Clinically-proven | Anti Breakage Test |
| to strengthen after 1 treatment | (see Section II) |
| Reduces breakage after 1 treatment | |
| Color safe | Color LAB Change with |
| | Washing Test (see Section V) |

### I. Curl Droop Test

The effect of a hair treatment on hold over the course of 8 hours by tress length was evaluated using the curl droop method.

The tested tresses, approximately 1 gram in weight and 8 inches in length with round bundle tabs, contained European medium brown straight hair. All testing was performed on hair procured from International Hair Importers & Products (Glendale, NY).

Eight (8) tresses were tested per treatment group.

Tresses were combed into a maximum length state for the baseline image to be taken. Subsequent images were taken after treatment (0 hours), and after one (1), two (2), four (4), and eight (8) hours of exposure to 60% relative humidity.

### Products

15% SLES Solution (Control)
Olaplex 4C Shampoo (Water/Aqua/Eau, Sodium Lauroyl Methyl Isethionate, Cocamidopropyl Hydroxysultaine, Sodium Lauroyl Glutamate, Coco-Betaine, Disodium Laureth Sulfosuccinate, Acrylates Copolymer, Cocamidopropyl Betaine, Cocamidopropylamine Oxide, Sodium Methyl Oleoyl Taurate, Decyl Glucoside, Bis-Aminopropyl Diglycol Dimaleate, Sodium Lauryl Sulfoacetate, Coco-Glucoside, Lauryl Glucoside, Panthenol, Trisodium Ethylenediamine Disuccinate, Ethylhexylglycerin, Polyquaternium-11, Sodium PCA, Pentasodium Triphosphate, Sodium Lactate, Arginine, Aspartic Acid, PCA, Citric Acid, Glycine, Alanine, Serine, Valine, Isoleucine, Proline, Threonine, Histidine, Phenylalanine, Phenoxyethanol, Chlorphenesin, Sodium Benzoate, Benzoic Acid, Fragrance (Parfum), Citral, Hexyl Cinnamal, Limonene)
Pro Curl System:
Step 1: Exemplary Bond Shaping Formulation (see “Exemplary Formulations” section)
Step 2: Exemplary Enhancing Formulation (see “Exemplary Formulations” section)
Step 3: Exemplary Bond Building Formulation (see “Exemplary Formulations” section)

### Treatments

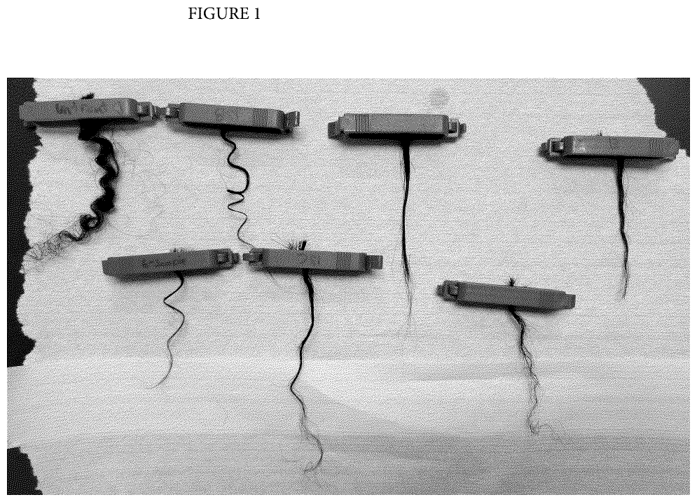

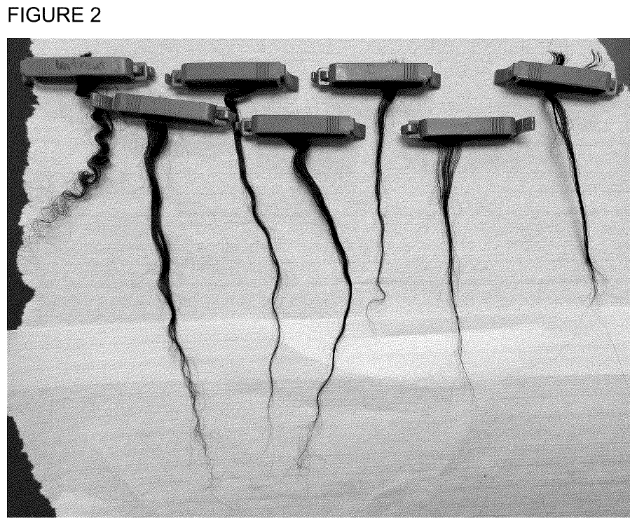

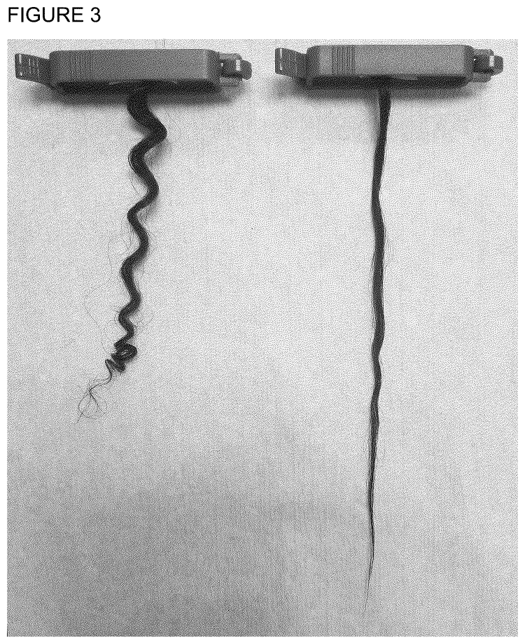

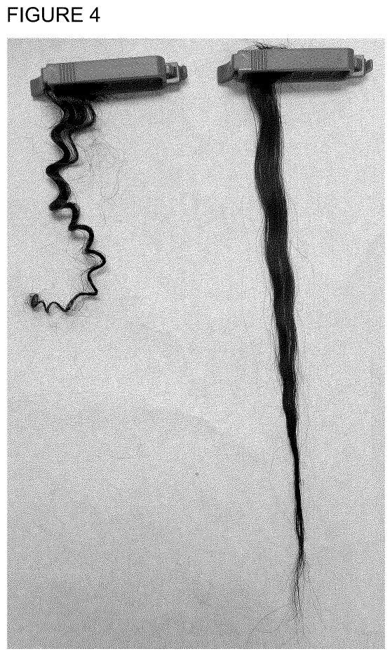

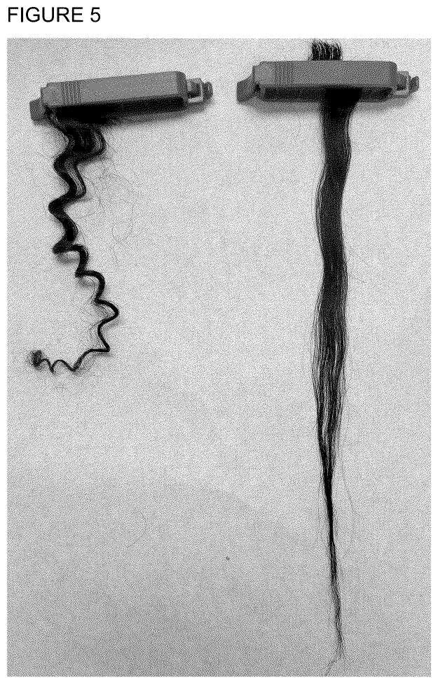

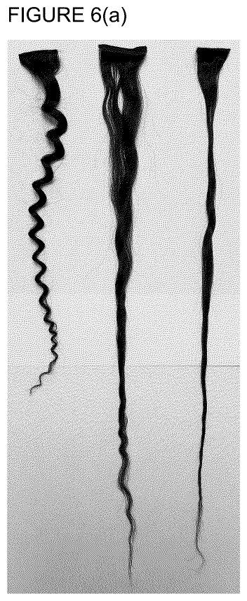

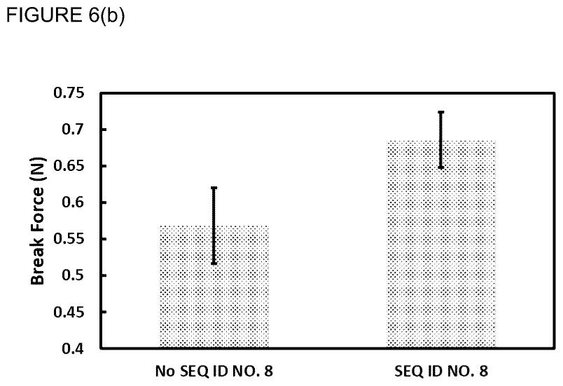

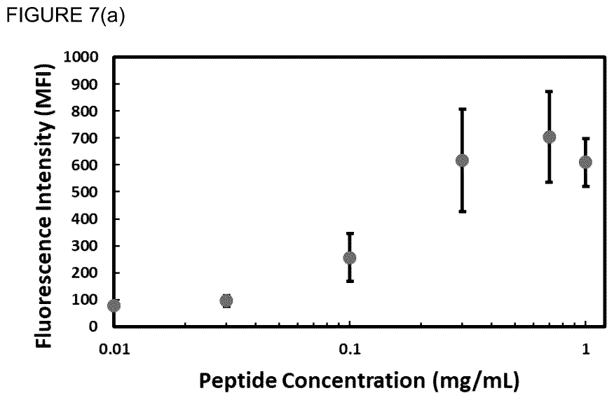

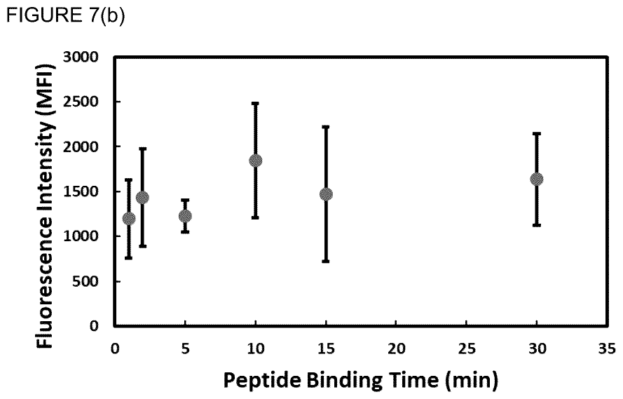

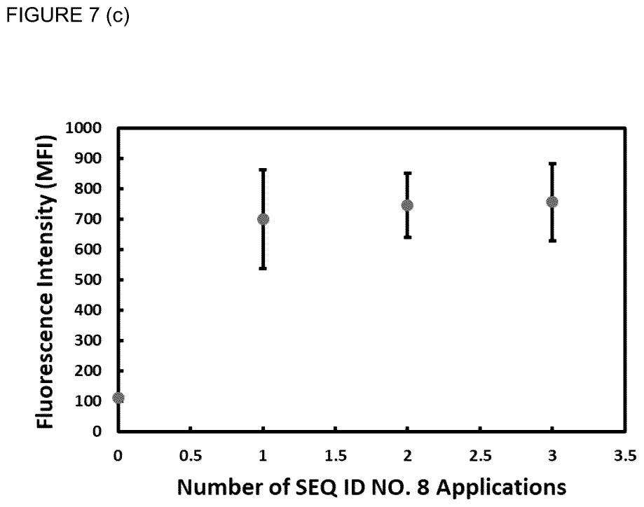

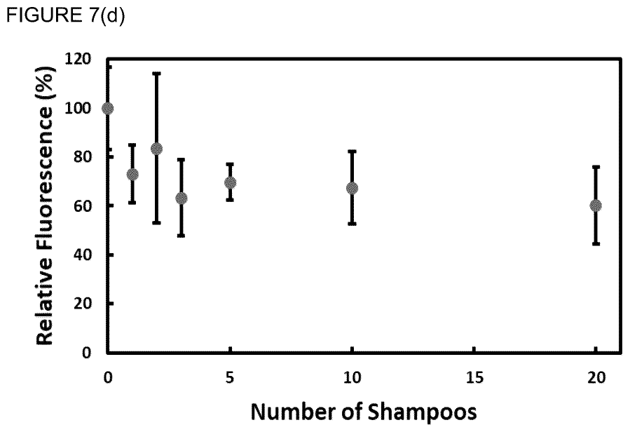

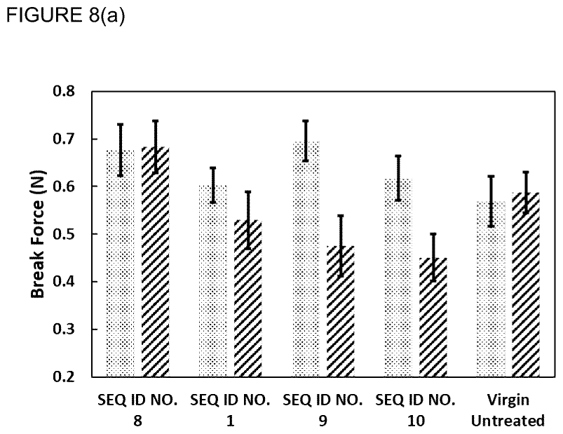

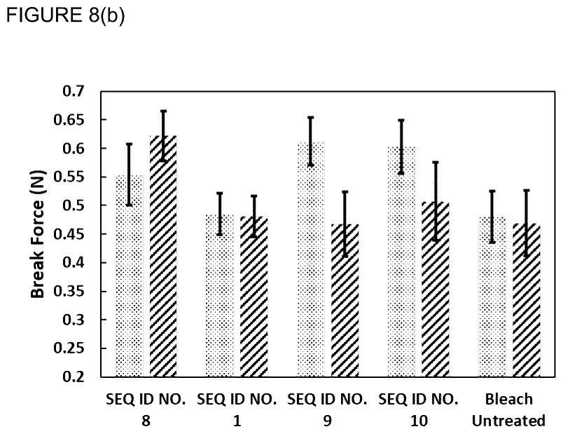

All tresses were standardized with 15% SLES Solution at 10% by weight (0.10 g±0.01 g for 1 g of dry hair) and left overnight to equilibrate under controlled ambient conditions (30% RH, 22° C.) prior to any treatment. Tresses were combed into a maximum length state for baseline imaging, then treated with their respective treatments outlined below. All treatments were performed using an Intellifaucet set at 40° C. with a controlled flow rate of 1.0 GPM. After treatments, tresses were assembled into the formation shown in FIG. 1 and left overnight in the controlled humidity chamber to equilibrate. Initial images were taken the next day then tresses were transferred to 60% RH and subsequent imaging was performed.

### Control

On damp hair, non-conditioning shampoo (15% SLES Solution) was applied at a dose of 10% by weight of the tress (0.10 mL/g of dry hair), massaged for 30 seconds, then rinsed for 30 seconds.

### Pro Curl System

4C Shampoo: On wet hair, shampoo was applied at 10% by weight of the tress (0.1 mL/g of dry hair, 0.4 mL on a 4-gram tress), massaged for 30 seconds, left in for 5 minutes, then rinsed for 30 seconds.

Step 1: On damp hair, product was applied at 20% by weight of the tress (0.2 mL/g of dry hair, 0.8 mL on a 4-gram tress), massaged for 30 seconds, and allowed to process for 10 minutes.

Step 2: From a distance of 6 inches, tress was sprayed three times (top, middle, bottom). The tress was flipped over. A total of 6 sprays were repeated on a 4-gram tress. The tress was allowed to process for 10 minutes.

Step 3: Product was applied at a dose of 15% by weight of the tress (0.15 mL/g of hair). The tress was massaged for 30 seconds and left in (no rinse).

Pro Curl System was allowed to process on the hair for 48 hours before testing.

### Testing Procedure

Testing involved the use of image analysis methods to track the increase in length of curled tresses after exposure to controlled humidity conditions. Treated hair tresses were wrapped in a specific conformation using in-house designed pegboards.

Tresses were allowed to dry overnight and reached equilibration at 30% RH. The tresses were removed from the pegboards and mounted on holders located in a bench-top humidity chamber. Once all tresses were hung on the board, the experiment began. The relative humidity was maintained at 60% for the initial image, and after one (1), two (2), four (4), and eight (8) hours. Eight replicate hair tresses were used for each sample to ensure statistical significance.

### Curl Droop Calculation

The length of individual tresses was determined using custom written software. The amount of curl droop for each tress was calculated using the following formula:

$$( ( {L}_{T} - {L}_{O} ) / ( {L}_{O} L\_ - {L}_{O} ) )$$

where L_{T}=Length at any given time, Lo=Initial time point Length of the tress, L_{O} L_=Length of the tress in its longest, straight position.

### Results

At all time-points measured, there was a statistical difference in percent curl droop when comparing the Pro Curl System to the Control: 15% SLES Solution. The Pro Curl System showed a lower percent curl droop, indicating its efficacy at retaining curl for up to 8 hours.

### II. Anti-Breakage Test

The manner by which products protect against breakage was demonstrated via repeated grooming.

Ten (10) 3 g, 8″ long, 1″ wide Curly multi-ethnic brown tresses (Walker type 3A), 9% bleached, were used per treatment group.

### Products

15% SLES (Control)
Olaplex 4C Shampoo (Water/Aqua/Eau, Sodium Lauroyl Methyl Isethionate, Cocamidopropyl Hydroxysultaine, Sodium Lauroyl Glutamate, Coco-Betaine, Disodium Laureth Sulfosuccinate, Acrylates Copolymer, Cocamidopropyl Betaine, Cocamidopropylamine Oxide, Sodium Methyl Oleoyl Taurate, Decyl Glucoside, Bis-Aminopropyl Diglycol Dimaleate, Sodium Lauryl Sulfoacetate, Coco-Glucoside, Lauryl Glucoside, Panthenol, Trisodium Ethylenediamine Disuccinate, Ethylhexylglycerin, Polyquaternium-11, Sodium PCA, Pentasodium Triphosphate, Sodium Lactate, Arginine, Aspartic Acid, PCA, Citric Acid, Glycine, Alanine, Serine, Valine, Isoleucine, Proline, Threonine, Histidine, Phenylalanine, Phenoxyethanol, Chlorphenesin, Sodium Benzoate, Benzoic Acid, Fragrance (Parfum), Citral, Hexyl Cinnamal, Limonene)
Pro Curl System:
Step 1—Exemplary Bond Shaping Formulation (see “Exemplary Formulations” section)
Step 2—Exemplary Enhancing Formulation (see “Exemplary Formulations” section)
Step 3—Exemplary Bond Building Formulation (see “Exemplary Formulations” section)

### Treatments/Applications

All treatment was performed using an Intellifaucet set at 40° C. with a controlled flow rate of 1.0 GPM. All tresses were standardized by washing with 15% SLES prior to treatment.

### Cell 1: 15% SLES (Control)

On wet hair, 15% SLES was applied at 10% by weight of the tress (0.1 ml/g of hair), massaged 30 sec, and rinsed 30 sec.

### Cell 2: Pro Curl System

Shampoo tress with Olaplex 4C Shampoo: On wet hair, shampoo was applied at 10% by weight of the tress (0.1 ml/g of hair), massaged 30 sec, left in 5 minutes, and rinsed 30 sec.

Step 1: On damp hair, Step 1 was applied at 20% by weight of the tress (0.2 ml/g of hair), massaged 30 sec, processed for 10 minutes, and Left in (no rinse).

Step 2: From a distance of 6 inches, tress was sprayed three times (top, middle, bottom). The tress was flipped over. A total of 6 sprays were repeated on a 4-gram tress. The tress was allowed to process for 10 minutes and left in (no rinse).

Step 3: Bond Building Formulation was applied at 15% by weight of the tress (0.15 ml/g of hair), massaged 30 sec, and left in (norinse).

Tresses were allowed to air dry and equilibrated for 48 hours at 60% RH prior to testing.

### Dry Repeated Grooming

After tresses were treated as outlined above, they were groomed using an automatic groomer for a total of 2,000 cycles. Fibers were collected and assessed after every 200 cycles.

### Anti-Breakage by Repeated Grooming

In this test, the number of broken fibers was recorded as a function of repeated combing/brushing strokes. Any treatment that reduced snags, entanglements and abrasion can help in substantially lowering the number of broken fibers. The methodology utilized a custom-built automated grooming device. It included a hollow rotating drum-like assembly, where four outer crossbars contained holders for mounting combs or brushes. These outer arms were detachable to allow for different holders to be mounted and experiments to be performed using a variety of combs or brushes. The four combs or brushes were mounted at 90° angles—allowing one complete drum revolution to comb (or brush) a tress four times. This entire set-up was replicated ten times in the horizontal direction allowing ten tresses to be combed simultaneously. Collection plates were located under each tress to save broken fiber fragments, while spacer plates on the rotating drum prevented cross contamination. For this experiment, there were eight replicates per cell. All experiments were performed after overnight equilibration of the hair at 60% RH at 22° C. This methodology is described in Evans & Park, A Statistical Analysis of Hair Breakage. II. Repeated Grooming Experiments, J.Cosmet.Sci., 61, 439-455, 2010.

### % Reduction in breakage Calculation

% Reduction in breakage was calculated using the following formula:

$$\% Reduction in breakage = \{ 1 - ( Breakage of treatment / Breakage of control ) \} \times 100$$

The calculated percent value was based solely on the average mean for each treatment.

### Standard Bleaching Protocol

Hair tresses were bleached using a 9% hydrogen peroxide solution at pH of 10.2. The tresses were left in contact with the bleach solution for 20 minutes under controlled temperature conditions (40° C.). At the end of this process, tresses were thoroughly rinsed under an intellifaucet set at 40° C. with a controlled flow rate of 1.0 GPM. The bleaching procedure was repeated two more times. Data Tukey Outlier Analysis was conducted.

### Results

There was a statistical reduction in breakage for Cell 2: Pro Curl Treatment compared with Control (15% SLES).

### III. Anti-Frizz Test

The frizz control properties of tresses under high (80%) relative humidity (RH) treated with a control and those treated with the Pro Curl System were compared and evaluated for immediate (0), eight (8), twenty-four (24), forty-eight (48), and seventy-two (72) hour testing.

The tested tresses, approximately 4 grams in weight and 8 inches in length, contained multi-ethnic frizzy hair (curly type 3B). All testing was performed on hair procured from International Hair Importers & Products (Glendale, NY).

Eight (8) tresses were tested per treatment group.

Tresses were combed into a maximum frizz state for the baseline image to be taken. Subsequent images were taken after treatment (0 hours), and after eight (8), twenty-four (24), forty-eight (48), and seventy-two (72) hours of exposure to 80% RH. Analysis was completed to compare the maximized baseline to the requested time points.

### Products

15% SLES Solution (Control)

Olaplex 4C Shampoo (Water/Aqua/Eau, Sodium Lauroyl Methyl Isethionate, Cocamidopropyl Hydroxysultaine, Sodium Lauroyl Glutamate, Coco-Betaine, Disodium Laureth Sulfosuccinate, Acrylates Copolymer, Cocamidopropyl Betaine, Cocamidopropylamine Oxide, Sodium Methyl Oleoyl Taurate, Decyl Glucoside, Bis-Aminopropyl Diglycol Dimaleate, Sodium Lauryl Sulfoacetate, Coco-Glucoside, Lauryl Glucoside, Panthenol, Trisodium Ethylenediamine Disuccinate, Ethylhexylglycerin, Polyquaternium-11, Sodium PCA, Pentasodium Triphosphate, Sodium Lactate, Arginine, Aspartic Acid, PCA, Citric Acid, Glycine, Alanine, Serine, Valine, Isoleucine, Proline, Threonine, Histidine, Phenylalanine, Phenoxyethanol, Chlorphenesin, Sodium Benzoate, Benzoic Acid, Fragrance (Parfum), Citral, Hexyl Cinnamal, Limonene)
Step 1: Exemplary Bond Shaping Formulation (see “Exemplary Formulations” section)
Step 2: Exemplary Enhancing Formulation (see “Exemplary Formulations” section)
Step 3: Exemplary Bond Building Formulation (see “Exemplary Formulations” section)

### Treatments

All treatments were performed using an Intellifaucet set at 40° C. with a controlled flow rate of 1.0 GPM. All tresses were initially standardized with 15% SLES Solution at 10% by weight of the tress (0.10 g±0.01 g for 1 g of dry hair) and left overnight to equilibrate under controlled ambient conditions (20% RH, 22° C.) prior to any treatment. Tresses were combed into a maximum frizz state for baseline imaging, then treated with their respective regimens outlined below. After treatments, tresses were reeqilibrated for 48 hours at 20% RH in the controlled humidity chamber. Initial images were taken after equilibration, then tresses were transferred to 80% RH and subsequent imaging was performed.

### Control

On damp hair, non-conditioning shampoo (15% SLES Solution) was applied at a dose of 10% by weight of the tress (0.10 mL/g of dry hair, 0.40 mL on a 4-gram tress). The tress was massaged for 30 seconds, then rinsed for 30 seconds.

### Pro Curl System

4 C Shampoo: On wet hair, shampoo was applied at 10% by weight of the tress (0.1 mL/g of dry hair, 0.4 mL on a 4-gram tress). The tress was massaged for 30 seconds, left in for 5 minutes, then rinsed for 30 seconds.
Step 1: On damp hair, product was applied at 20% by weight of the tress (0.2 mL/g of dry hair, 0.8 mL on a 4-gram tress). The tress was massaged for 30 seconds and allowed to process for 10 minutes.
Step 2: From a distance of 6 inches, tress was sprayed three times (top, middle, bottom). The tress was flipped over. A total of 6 sprays were repeated on a 4-gram tress. The tress was allowed to process for 10 minutes.
Step 3: Product was applied at a dose of 15% by weight of the tress (0.15 mL/g of hair), massaged for 30 seconds and left in (no rinse).

### Testing Procedure

Images were first taken of the frizzy hair tresses in their native state. The tresses were then treated with the appropriate products and moved to 80% relative humidity (RH) where further images were collected initially and after eight (8), twenty-four (24), forty-eight (48), and seventy-two (72) hours. Eight tresses were evaluated per sample.

### Frizz Reduction Calculation

The frizz of the hair tresses was determined by pixel count. The percent reduction in frizz of a tress was given by the equation:

$$\% Reduction in Frizz = \{ ( Max Frizz - Treatment Frizz ) / Max Frizz \} \times 100$$

### Results

At all time-points measured, there was a statistical difference in percent frizz reduction when comparing tresses treated with the Pro Curl System to tresses treated with the Control: 15% SLES Solution. Compared to the Control, the Pro Curl System showed a higher percent frizz reduction immediately after the treatment and 48-hour processing period (no washes), and for 10, 12, and 15 washes post-treatment and processing period. The reduction in frizz measured in tresses treated with the Pro Curl System showed the effectiveness of the treatment at reducing frizz for up to 72 hours at high humidity. The frizz reduction benefit lasted through 10, 12 and 15 wash cycles after treatment.

### IV. Wet Combing Test

The smoothness properties, in the wet state, for bleached tresses treated with the Pro Curl System treatment below were evaluated and compared against control shampoo alone, via Wet combing (Garcia Method).

Eight (8) Curly Multi-Ethnic Brown, 6% Bleached, hair tresses (3.0 g, 8″ length, 1″ wide) were used per treatment group.

### Products

Suave Non-Conditioning Shampoo (Control)
Olaplex No. 4C Bond Maintenance Clarifying Shampoo
Pro Curl System:
Step 1: Exemplary Bond Shaping Formulation (see “Exemplary Formulations” section)
Step 2: Exemplary Enhancing Formulation (see “Exemplary Formulations” section)
Step 3: Exemplary Bond Building Formulation (see “Exemplary Formulations” section)

### Treatments

All treatments were performed using an Intellifaucet set at 40° C. with a controlled flow rate of 1.0 GPM. All tresses were standardized by washing with 15% SLES prior to treatments.

### Cell 1: Suave Non-Conditioning Shampoo (Control)

On damp hair, applied 10% by weight of the tress (0.1 mL/g of hair), massaged for 30seconds, rinsed for 30 seconds.

### Cell 2: Pro Curl System

Olaplex No. 4C Bond Maintenance Clarifying Shampoo: On wet hair, applied at 10% by weight of the tress (0.1 ml/g of hair), massaged 30 seconds, left in 5 minutes, rinsed 30 seconds.

Pro Curl System Step 1: On damp hair, applied at 20% by weight of the tress (0.2 ml/g of hair), massaged 30 seconds, processed for 10 minutes. Left in, no rinse.

Pro Curl System Step 2: On damp hair, from a distance of 6 inches, sprayed tress 3 times (top, middle and bottom), flipped over, and repeated for a total of 6 sprays on a 3 g tress. Processed for 10 minutes. Left in, no rinse.

Pro Curl System Step 3: On damp hair, applied at 15% by weight of the tress (0.15 ml/g of hair), massaged 30 seconds. Left in, no rinse.

Testing: Wet Combing Method
1. Eight (8) tresses (each 3.0 g, 8 in in length) were used per treatment group.
2. Tresses were treated as outlined above and then immediately tested wet.
3. Six (6) measurements were taken per tress using an Instron tensile tester to evaluate product performance.

### Wet Combing Force

A common and consumer-relevant approach for measuring lubrication following application of a hair care product involved an instrumental combing experiment. Testing involved use of an Instron tensile tester to measure frictional forces while a hair tress was pulled through a comb.

Such experiments were performed in accordance with the widely used method proposed by Garcia & Diaz (JSCC, 27, (1976) 379-398—Combability Measurements on Hair). Combing experiments were performed in the wet state after treatment. Six combing strokes were performed per tress, while eight replicate hair tresses were used per sample to ensure statistical relevance.

### Standard Bleaching Protocol

Some tresses were subject to a bleaching protocol prior to testing. Hair tresses were bleached using a 6% hydrogen peroxide solution at pH of 10.2. The tresses were left in contact with the bleach solution for 40 minutes under controlled temperature conditions (40° C.). At the end of this process, tresses were thoroughly rinsed under an intellifaucet set at 40° C. with a controlled flow rate of 1.0 GPM.

### Percent Reduction Calculation

Percent reduction in Combing Forces was calculated as follows:

$$\% Reduction in combing force = \{ 1 - ( combing force of treatment / combing force of control ) \} \times 100$$

The calculated percent Reduction in combing force can be calculated solely on the average mean for each treatment.

### Results

When compared to the control, the Pro Curl System showed a statistical difference in wet combing force.

### V. Color LAB Change with Washing Test

The manner by which products change the color of hair was demonstrated with repeated washing via Hunterlab Colorimeter.

Color (L*A*B) change with washing Substrate: 3 g, 8″ long, 1″ wide Curly multi-ethnic brown tresses (Walker type 3A), 6% bleached and colored with commercially available auburn dye. The bleaching and color treatment protocol are provided below.

Ten (10) tresses were tested per treatment group.

After treatment and after ten (10), twelve (12) and fifteen (15) wash cycles.

### Products

### Cell 1:

Control (SLES 15% to mimic shampoo+water to mimic Step 1, Step 2, and Step 3)

### Cell 2:

Olaplex 4C Shampoo (Water/Aqua/Eau, Sodium Lauroyl Methyl Isethionate, Cocamidopropyl Hydroxysultaine, Sodium Lauroyl Glutamate, Coco-Betaine, Disodium Laureth Sulfosuccinate, Acrylates Copolymer, Cocamidopropyl Betaine, Cocamidopropylamine Oxide, Sodium Methyl Oleoyl Taurate, Decyl Glucoside, Bis-Aminopropyl Diglycol Dimaleate, Sodium Lauryl Sulfoacetate, Coco-Glucoside, Lauryl Glucoside, Panthenol, Trisodium Ethylenediamine Disuccinate, Ethylhexylglycerin, Polyquaternium-11, Sodium PCA, Pentasodium Triphosphate, Sodium Lactate, Arginine, Aspartic Acid, PCA, Citric Acid, Glycine, Alanine, Serine, Valine, Isoleucine, Proline, Threonine, Histidine, Phenylalanine, Phenoxyethanol, Chlorphenesin, Sodium Benzoate, Benzoic Acid, Fragrance (Parfum), Citral, Hexyl Cinnamal, Limonene)
Pro Curl System:
Step 1—Exemplary Bond Shaping Formulation (see “Exemplary Formulations” section)
Step 2—Exemplary Enhancing Formulation (see “Exemplary Formulations” section)
Step 3—Exemplary Bond Building Formulation (see “Exemplary Formulations” section)

### Treatments

All treatments were performed with an Intellifaucet set at 40° C. with controlled flow rate of 1.0 GPM. After treatments, tresses were left to equilibrate overnight under controlled ambient conditions before testing.

### Cell 1: Control

SLES 15% (mimic shampoo): On wet hair, applied SLES (15%) at 10% by weight of the tress (0.1 ml/g of hair), massaged 30sec, left in 5 minutes, rinsed 30 sec.

Water (mimic Step 1): On damp hair, applied water at 20% by weight of the tress (0.2 ml/g of hair), massaged 30 sec, processed for 10 minutes. Left in, no rinse.

Water (mimic Step 2): From a distance of 6 inches, sprayed tress three times (top, middle, bottom), flipped over, and repeated for a total of 6 sprays on a 3 g tress. Processed for 10 minutes. Left in, no rinse.

Water (mimic Step 3): Applied water at 15% by weight of the tress (0.15 ml/g of hair), massaged 30 sec. Left in, no rinse.
Notes: 1. Tresses were allowed to air dry and equilibrate for 48 hours at 60% RH prior to testing.
2. For repeated shampoo treatments for color fade, 15% SLES was used.

On wet hair, SLES (15%) was applied at 10% by weight of the tress (0.1 ml/g of hair), massaged 30 sec, left in 5 minutes, rinsed 30 sec.

### Cell 2: Pro Curl System

Shampoo tress with Olaplex 4C Shampoo: On wet hair, applied shampoo at 10% by weight of the tress (0.1 ml/g of hair), massaged 30 sec, left in 5 minutes, rinsed 30 sec.
Step 1: On damp hair, applied Step 1 at 20% by weight of the tress (0.2 ml/g of hair), massaged 30 sec, processed for 10 minutes. Left in, no rinse.
Step 2: From a distance of 6 inches, sprayed tress three times (top, middle, bottom), flipped over, and repeated for a total of 6 sprays on a 3 g tress. Processed for 10 minutes. Left in, no rinse.
Step 3: Applied step 3 at 15% by weight of the tress (0.15 ml/g of hair), massaged 30 sec. Left in, no rinse.
Notes: 1. tresses were allowed to air dry and equilibrate for 48 hours at 60% RH prior to testing.
2. For repeated shampoo treatments for color fade, 15% SLES was used.

On wet hair, SLES (15%) was applied at 10% by weight of the tress (0.1 ml/g of hair), massaged 30 sec, left in 5 minutes, rinsed 30 sec.

### Testing Procedure

All tresses were dyed using L'Oreal Preference shade 4R Dark Auburn dye. A reproducible dye protocol was required to ensure that all tresses reach a comparable initial state. The procedure was provided below. After drying and equilibrating over-night at 60% relative humidity (RH) & 22° C., baseline readings were obtained. Based on the readings, tresses were distributed between the sets in a manner that generated the closest average L, a, and b values when comparing the sets. This allowed a better comparison of average color loss after washing as all sets begun with similar values. Tresses were treated, as outlined above, in intervals of ten (10), twelve (12) and fifteen (15) washes. Tresses were allowed to dry and equilibrate overnight between each wash interval. Color measurements were taken at the baseline, 10×, 12× and 15× wash cycles.

### Color Fade

All the dyed hair tresses were labeled to allow for identification. Baseline L, a, b measurements were taken using a Hunter Lab UltraScan VIS colorimeter to characterize the initial color of hair. Technical hair color was often quantified using the CIELAB L, a, b system. That color is represented in a 3-dimensional matrix where “L” refers to the lightness on a scale of 0 to 100, “a” denotes the red-green color range (positive value denotes higher red) and “b” represents the yellow-blue color range (positive value denotes higher yellow).

Changes in the color of the tress can be evaluated by calculating the difference in these parameters relative to a reference state (i.e. ΔL*, Δa*, and Δb*). In addition, it is common to report an overall color change, ΔE. That is, ΔE=√[ΔL*2+Δa*2+Δb*2]. Ten (10) color measurements were performed on each tress.

### Bleaching Procedure

Hair was bleached using a standard bleaching technique that improved the sensitivity of instrumental combing experiments. This involved treating hair tresses with a skeletal 6% hydrogen peroxide solution at a pH adjusted to 10.2. This procedure is described below.
1. Placing virgin tresses in a Pyrex cooking dish.
2. Pouring the prepared 6% hydrogen peroxide solution (heated to 40° C. and pH adjusted to 10.2 over the hair at a dosage of 500 ml per 40 tresses.
3. Allowing the tresses to process for 40 minutes under controlled temperature conditions (40° C.) with regular agitation to ensure uniform bleaching.
4. After processing, introducing the tresses to a Pyrex dish containing clean water for the purpose of rinsing. Performing this twice.
5. Rinsing thoroughly under an intellifaucet set at 40° C. with a controlled flow rate of 1.0 GPM.

### Standard Color Treatment Procedure

The hair tresses were color treated according to the procedure below with L'Oreal Preference shade 4R.
1. Combining color components according to the manufacturer's instructions and mixing thoroughly.
2. Adding 10 grams of mix to each hair tress using a brush (5 g on each side) and massaging thoroughly into tress.
3. Positioning tresses on a hot plate with temperature maintained at 27° C.
4. Allowing the color to process on the hair for 20 minutes at 27° C.
5. Turning tresses over after 10 min (halfway) and massaging to allow for even coloring.
6. Rinsing out under an Intellifaucet for 2 minutes (or until water runs clear) at 40° C. and 1.0 GPM flow rate.
7. Allowing tresses to rest for at least 12 hours prior to taking post color readings.

### Results

At the ten (10), twelve (12) and fifteen (15) treatment intervals, there was no statistical difference between Control (Cell 1) and Pro Curl System (Cell 2). This shows that the Pro Curl System is safe for color-treated hair.

We claim:

1. A kit for defining curls comprising:
(a) a bond shaping formulation comprising a peptide; and
(b) a bond building formulation comprising bis-aminopropyl diglycol dimaleate.

2. The kit of claim 1, wherein the peptide comprises an amino acid sequence of any one of SEQ ID NOS: 3-SEQ ID NOS: 17, SEQ ID NOS: 18-SEQ ID NOS: 109, or a fragment or variant of any one of SEQ ID NO:1 and SEQ ID NOS: 3-SEQ ID NOS: 17, SEQ ID NOS: 18-SEQ ID NOS: 109.

3. The kit of claim 1, wherein the peptide is SEQ ID NO:8 or a variant thereof with at least 70% sequence identity thereto.

4. The kit of claim 1, wherein the peptide consists of SEQ ID NO:8.

5. The kit of claim 1, wherein the peptide is present in an amount from about 0.01 to about 0.1% wt of the bond shaping formulation, and the binding agent is present in an amount from about 0.01 wt % to about 1 wt %, from about 0.01 wt % to about 0.5 wt %, from about 0.01 to about 0.1 wt %, or from about 0.01 to about 0.05 wt % of the bond building formulation.

6. The kit of claim 1, further comprising an enhancing formulation comprising water, an alcohol (optionally propanediol), a chelating agent, a surfactant, an emollient, an emulsifier, and optionally a fragrance, optionally wherein the total amount of the chelating agent, surfactant, emollient, and emulsifier is less than 0.1 wt % of the enhancing formulation.

7. The kit of claim 6, wherein the enhancing formulation comprises water, propanediol, caprylyl/capryl glucoside, sodium cocoyl glutamate, citric acid, trisodium ethylenediamine disuccinate, polyglyceryl-6 oleate, glyceryl caprylate, and sodium surfactin.

8. A method for defining curls, comprising:
(1) applying a bond shaping formulation to hair, wherein the bond shaping formulation comprises a peptide; and
(2) applying a bond building formulation to the hair, wherein the bond building formulation comprises bis-aminopropyl diglycol dimaleate.

9. The method of claim 8, wherein the peptide comprises an amino acid sequence of any one of SEQ ID NOS: 3-SEQ ID NOS: 17, SEQ ID NOS: 18-SEQ ID NOS: 109, or a fragment or variant of any one of SEQ ID NO:1 and SEQ ID NOS: 3.-SEQ ID NOS: 17, SEQ ID NOS: 18-SEQ ID NOS: 109.

10. The method of claim 8, wherein the peptide is SEQ ID NO:8 or a variant thereof with at least 70% sequence identity thereto, optionally wherein the peptide consists of SEQ ID NO:8.

11. The method of claim 8, further comprising, in step (1) or subsequent to step (1), step (i) raking through the hair in a downward motion to ensure the hair is fully saturated with the bond shaping formulation.

12. The method of claim 8, wherein the bond shaping formulation is left on the hair for a time period of about 1 min to about 20 mins or from about 5 mins to about 15 mins, such as about 10mins, following step (1).

13. The method of claim 8, further comprising, subsequent to step (1) and prior to step (2), step (1a) applying an enhancing formulation to the hair, wherein the enhancing formulation comprises water, an alcohol (optionally propanediol), a chelating agent, a surfactant, an emollient, an emulsifier, and optionally a fragrance, and
optionally wherein step (1a) is performed one or more times during step (2).

14. The method of claim 13, wherein in step (1a), the enhancing formulation is sprayed to saturate the hair with the enhancing formulation.

15. The method of claim 13, further comprising shaping the hair, such as by squeezing, compressing, or scrunching, to define the curls during step (1a) and/or subsequent to step (1a).

16. The method of claim 13, wherein the enhancing formulation is left on the hair for a time period of about 1 minute to about 20 minutes or from about 5 minutes to about 15 minutes, such as about 10 minutes, following step (1a).

17. The method of claim 8, further comprising,
(i) prior to step (1), step (3) rinsing the hair with water or washing the hair with a shampoo;
(ii) subsequent to step (2), step (4) shaping the curls into a desired shape, optionally with a curl defining tool, such as curlers, rollers, curling rod, and/or a curl defining brush; and/or
(iii) subsequent to step (2), step (5) drying the hair.

18. The method of claim 8, wherein subsequent step (2), the hair is not rinsed or washed for at least 24 hours, at least 36 hours, at least 48 hours, at least 60 hours, or at least 72 hours.

19. The method of claim 8, wherein following step (2), and following drying of the hair,
(i) the curls are shinier, bouncier, and/or more defined as compared to untreated hair from the same individual, by visual inspection, and/or
(ii) the hair has reduced frizz compared to untreated hair from the same individual or hair from the same individual that is washed with a non-conditioning shampoo, such as 15% SLES solution.

20. The method of claim 8, wherein following step (2),
(iii) the hair is stronger compared to untreated hair from the same individual or hair from the same individual that is washed with a non-conditioning shampoo, such as 15% SLES solution; and/or
(iv) the hair has reduced breakage compared to untreated hair from the same individual or hair from the same individual that is washed with a non-conditioning shampoo, such as 15% SLES solution.
